(19) United States
(12) Patent Application Publication
(10) Pub. No.: US 2015/0166500 A1
(43) Pub. Date: Jun. 18, 2015

(54) Compounds and Methods for Catalytic Directed ortho Substitution of Aromatic Amides and Esters

(71) Applicant: Queen's University at Kingston, Kingston (CA)

(72) Inventors: Yigang Zhao, Guelph (CA); Victor A. Snieckus, Kingston (CA)

(21) Appl. No.: 14/541,297

(22) Filed: Nov. 14, 2014

Related U.S. Application Data
(63) Continuation of application No. 13/479,942, filed on May 24, 2012, now abandoned.
(60) Provisional application No. 61/490,966, filed on May 27, 2011.

(51) Int. Cl. C07D 307/68; C07C 63/36; C07C 233/65

(52) U.S. Cl. CPC C07D 307/68; C07C 233/65; C07C 63/36

(57) ABSTRACT

Methods are described for efficient and regioselective reactions that are Ru-catalyzed and either (i) amide-directed C—H, C—N, C—O activation/C—C bond forming reactions, (ii) ester-directed C—O and C—N activation/C—C bond forming reactions, or (iii) amide-directed C—O activation/hydrodemethoxylation reactions. All of these reactions of directed C—H, C—N, C—O activation/coupling reactions establish a catalytic base-free DoM-cross coupling process at non-cryogenic temperature. High regioselectivity, yields, operational simplicity, low cost, and convenient scale-up make these reactions suitable for industrial applications. Many previously unknown amide-substituted or ester-substituted aryl and heteroaryl compounds are presented with synthetic details also provided.

### CROSS-REFERENCE TO RELATED APPLICATIONS

This application claims the benefit of the filing date of U.S. Provisional Patent Application No. 61/490,966 filed on May 27, 2011, the contents of which are incorporated herein by reference in their entirety.

### FIELD OF THE INVENTION

The field of the invention is a method to eliminate a substituent of an aryl substrate that is in an ortho position to a tertiary amide or ester ortho-directing group, and in some embodiments to form a C—C bond between aryl substrates and aryl and/or aliphatic substituents whereby the substituents bond at an ortho position relative to an ester or tertiary amide ortho-directing group. The field of the invention includes compounds that have been made by such methods.

### BACKGROUND OF THE INVENTION

Transition metal-catalyzed cross coupling reactions are arguably the most important C—C bond formation tools in organic synthesis in last 40 years (Corbet, J. P. et al., Chem. Rev. 2006, 106, 2651-2710; de Meijere, A.; Diederich, F.; (Eds) Metal-Catalyzed Cross-Coupling Reactions (2nd Edition); Wiley: Weinheim, 2004; and Beller, M.; Bolm, C.; (Eds) Transition Metals for Organic Synthesis; Wiley: Weinheim, 2004). Of these, aryl-alkene and aryl-aryl sp²-sp² cross couplings, such as the Mizoroki-Heck, Suzuki-Miyaura, Negishi, Migita-Stille and Kumada-Corriu cross couplings discovered in the 1970s, have been well-explored and broadly used for constructing C—C bonds. Most of these reactions involve cleavage of carbon-halogen and carbon-pseudohalogen bonds with transition metals (mostly Pd and Ni) and coupling with organometallic reagent species C—B, C—Zn, C—Sn and C—Mg in the Suzuki-Miyaura, Negishi, Migita-Stille and Kumada-Corriu cross couplings respectively. These couplings, in which both of aryl halides and organometallic reagents are required and which are called traditional cross couplings, generate stoichiometric amounts of halogen ions and metal species as undesired by-products which, except for boron, are ecologically harmful. Since the seminal work of Murai (Mural, S.; (Ed.). Topics in Organometallic Chemistry 1999, 3, Springer: New York.), chemists have tried to develop cross coupling reactions which originate from direct activation of unreactive bonds, especially C—H, C—O, C—N bonds which are among the most abundant bonds in organic molecules. Such reactions would be powerful synthetic strategies for C—C bond formation and could establish convenient, economical and green alternatives to traditional cross coupling processes.

### C—H Bond Activation and Cross Coupling Via Ketone-Directing

In 2003, Mural, Chatani, Kakiuchi and co-workers reported a new type of C—H bond arylation in the Ru-catalyzed coupling of ketones with organoboronates to give biaryls in good yields (Kakiuchi, F.; Kan, S.; Igi, K.; Chatani, N.; Murai, S. J. Am. Chem. Soc. 2003, 125, 1698-1699). The catalyst (RuH₂(CO)(PPh₃)₃) and solvent (toluene) are employed in this process. Both ortho C—H bonds are activated in an acetophenone substrate to give 2,6-diaryl products. Notably, a bulky t-butyl ketone (phenyl pivaloyl ketone) was used to avoid having two ortho C—H bond activation potentials involved in the reaction and therefore only one C—H activation proceeded. Electron donating groups and electron withdrawing groups including Me, CF₃, F, NMe₂ and OMe in both of starting materials were tolerated. However, at least 2:1 ratio of ketone:organoboronate was required for high yield coupling due to the existence of a reduction reaction, a feature which decreases the utility of the reaction for expensive and precious ketone substrates.

To overcome the above deficiency, aliphatic ketones such as pinacolone and acetone, which are more reactive than aryl ketones, were introduced as solvent to act as hydride scavenger from Ru—H generated by Ru insertion into the ortho-C—H bond of aromatic ketones. After this improvement, using an almost 1:1 ratio of aromatic ketone and organoboronate partners, the coupling reaction proceeded in good to excellent yields (Kakiuchi, F.; Matsuura, Y.; Kan, S.; Chatani, N. J. Am. Chem. Soc. 2005, 127, 5936-5945).

### C—O Bond Activation and Cross Coupling

Reductive aryl C—O bond cleavage in derivatives such as C—OTf, C—OAc, C—OPiv, C—OCONEt₂, C—OCO₂Bu-t and C—OSO₂NMe₂, are significant recent reactions in the organic chemist's tool box (de Meijere, A.; Diederich, F.; (Eds) Metal-Catalyzed Cross-Coupling Reactions (2nd Edition); Wiley: Weinheim, 2004; Guan, B. T. et al., J. Am. Chem. Soc. 2008, 130, 14468-14470; Li, B. J. et al., J. Angew. Chem. Int. Ed. 2008, 47, 10124-10127; Quasdorf, K. W. et al., J. Am. Chem. Soc. 2008, 130, 14422-14423; Quasdorf, K. W. et al., J. Am. Chem. Soc. 2009, 131, 17748-17749; and Antoft-Finch, A. et al., J. Am. Chem. Soc. 2009, 131, 17750-17752). Of these, reductive C—OTf bond cleavage has received broad application. Furthermore, these C—O functional groups serve as complementary cross coupling partners to aryl halides, allowing consideration of alternative phenol-derived processes to a halide, can directly undergo Suzuki cross coupling with organoboron partners. However, some drawbacks of these methodologies remain: i) all functional groups are characterized by at least modest or strong electron-withdrawing groups (EWGs), e.g., Tf, Ac, Piv, CONEt₂, CO₂Bu-t and SO₂NMe₂, for assisting oxidative addition by transition metal catalysts; ii) require expensive pre-preparation such as synthesis of aryl triflates from phenols with triflic anhydride. Considering the broad and commercial availability of aryl ethers, a discovery of a transition metal process for C—OMe bond cleavage would provide a convenient and powerful method for cross coupling.

In 2004, Kakiuchi-Chatani-Murai's group discovered a new type of C—O bond cleavage of aryl ethers by Ru-catalysis under chelation assistance (Kakiuchi, F. et al. J. Am. Chem. Soc. 2004, 126, 2706-2707). The new reaction involves Ru-catalyzed ketone-directed C—OMe bond activation and Suzuki-type C—C cross coupling with organoboronates. The scope for organoboroneopentylates was examined and a variety of functional groups in the arylboronates (Me, vinyl, OMe, F and CF₃) were found to be compatible. Both of C—H and C—O activation/coupling reactions occurred simultaneously when 2-methoxy acetophenone was employed. In order to avoid undesired C—H activation, a bulky t-butyl ketone was used for blocking the C—H activation by steric effects.

### C—N Bond Activation and Cross Coupling

Aryl C—N bonds have high bond dissociation enthalpies. Among the abundant bonds in organic molecules, the aromatic C—N bond is an unreactive or difficult-to-cleave bond for organic synthesis manipulation. As part of research in chelation-assisted reactions of aryl ketones with organoborates, Kakiuchi and co-workers discovered the Ru-catalyzed C—N bond activation/Suzuki-type cross coupling reaction (Ueno, S.; Chatani, N.; Kakiuchi, F. J. Am. Chem. Soc. 2007, 129, 6098-6099). The reaction is carried out under conditions similar to those used for the directed C—OMe bond activation/cross coupling reactions (Ueno, S.; Mizushima, E.; Chatani, N.; Kakiuchi, F. J. Am. Chem. Soc. 2006, 128, 16516-16517). The ketone directing group and RuH₂(CO)(PPh₃)₃ catalysis play a key role in the necessary C—NR₂ activation, in which the coordination of Ru(O) to the ketone carbonyl assists Ru(O) insertion into the C—NR₂ bond analogous to the C—OMe insertion process. Similarly, the bulky t-butyl is used to avoid the undesired C—H activation as in the C—OMe activation case.

### Combined DoM-Transition Metal-Catalyzed Cross Coupling Reaction

Directed ortho metalation (DoM) reactions have become an important synthetic tool for aromatic ring C—H functionalization in organic synthesis and is widely used in research and in industry (Snieckus, V. Chem. Rev. 1990, 90, 879-933; Hartung, C. G. et al., Modern Arene Chemistry 2002, 330-367. Wiley: Weinheim; and Snieckus, V., et al., Handbook of C—H Transformations 2005, 1, 106-118, 262-264. Wiley: Weinheim). Furthermore, a combined DoM-cross coupling strategy (see Scheme 1, FIG. 1) plays an important role in C—C bond forming reactions via DoM chemistry (Anctil, E. J. G., et al., J. Organomet. Chem. 2002, 653, 150-160; and Anctil, E. J. G., et al., Metal-Catalyzed Cross-Coupling Reactions (2nd Ed) 2004, 2, 761-813. Wiley: Weinheim). This tactic has links to Suzuki-Miyaura, Kumada-Corriu, Negishi and Migita-Stille cross coupling processes, of which the DoM-Suzuki reaction is considered to be the most efficient and practical. This strategy is suitable for construction of not only aryl-aryl but also heteroaryl-heteroaryl and their mixed systems.

However, requisitions such as harsh conditions (e.g., low temperature and strong base, usually −78° C. and BuLi) have limited the applications of DoM chemistry. The necessity of stoichiometric or excess amounts of base is still a drawback in these reactions.

### Amide-Directed C—H, C—O, C—N Bond Activation/Cross Couplings

To the best of our knowledge, only two examples of tertiary amide mediated catalytic C—H bond functionalization have been reported: the first case involves the Ru₃(CO)₁₂-catalyzed silylation of a C—H bond of furan 2-carboxamide 1 (see below) (Kakiuchi, F. et al., Chem. Lett. 2000, 750-751). This reaction was carried out to test the amide-directed C—H activation/olefin coupling reaction which did proceed to give 2 but in very low yield, the major product being the 3-TMS derivative 3, a mechanistically interesting result. The second example is the Pd(OAc)₂-catalyzed C—H activation/arylation of the thiophene amide 4 which leads to products 5 and 6 whose formation evidently occurs by non-ortho and ortho-directing group activation reactions (see below) (Okazawa, T. et al., J. Am. Chem. Soc. 2002, 124, 5286-5287). In addition, it has been reported that a tertiary benzamide was examined for an amide-directed C—H activation/arylation under Pd(OAc)₂/PPh₃/Cs₂CO₃ catalysis condition but that this reaction failed to give coupled product (Kametani, Y. et al., Tetrahedron Lett. 2000, 41, 2655-2658).

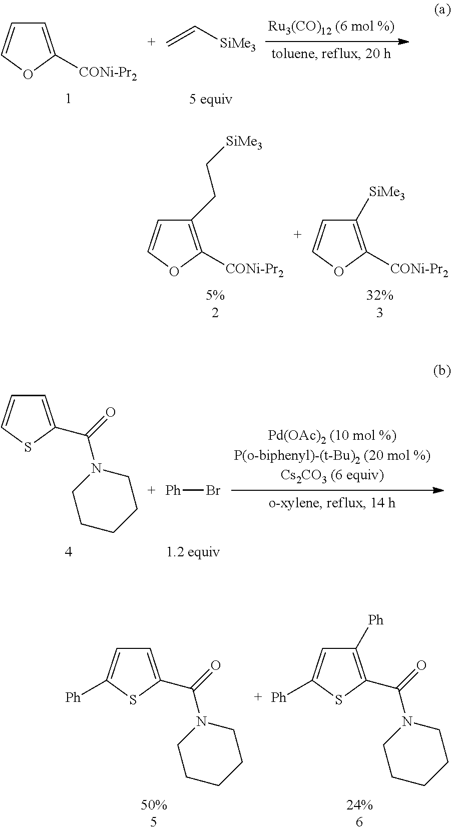

Amide-directed C—O and C—N bond functionalizations are not previously known. The discovery of an amide-directed catalytic arylation reaction will fill a need: a catalytic base-free DoM-cross coupling process at non-cryogenic temperatures.

### SUMMARY OF THE INVENTION

In a first aspect the invention provides a method of forming a carbon-carbon (C¹—C²) bond between an aryl ring carbon (C¹) and an addition moiety carbon (C²), comprising combining in an inert atmosphere to form a reaction mixture: (i) an aryl substrate comprising a substituent which is an ester or amide ortho-directing group in an ortho position to a departing substituent, wherein for amide directing groups, the departing substituent is bonded to an aryl ring carbon (C¹) through a hydrogen, oxygen, or nitrogen atom, and wherein for ester directing groups, the departing substituent is bonded to an aryl ring carbon (C¹) through an oxygen or nitrogen atom; (ii) a boronate comprising a boron bonded to an addition moiety through a carbon (C²); and (iii) a catalytic amount of a ruthenium or rhodium complex; allowing reaction to proceed under suitable conditions of temperature and pressure for an appropriate reaction time to produce a product that is a modified form of the aryl substrate, wherein the modification is that the addition moiety has replaced the departing substituent and is bonded through its carbon (C²) to the ring carbon (C¹), and is ortho to the ester or amide ortho-directing group.

In embodiments of this aspect the aryl substrate is heteroaryl. In certain embodiments heteroaryl is furanyl, pyridyl, pyrimidinyl, indolyl, or thiophenenyl. In certain embodiments aryl comprises fused aryl rings. In certain embodiments fused aryl rings are naphthylene, anthracene, or phenanthrene. In embodiments of this aspect the boronate is

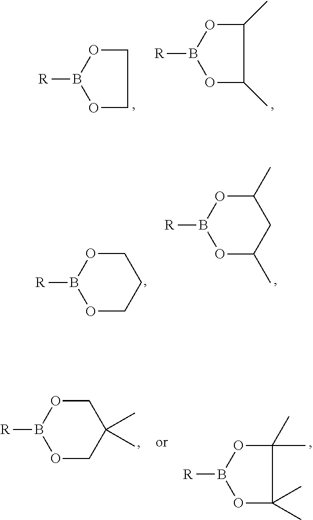

wherein addition group “R” is an aryl, aliphatic, aliphatic-aryl, or aryl-aliphatic moiety. In certain embodiments, the boronate is

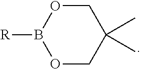

In another embodiment of this aspect the suitable conditions of temperature comprises heating to a temperature range from about 80° C. to about 250° C. In some embodiments, the said suitable conditions of temperature comprises heating to about 120° C. In some embodiments, when the ortho-directing group is ester and the aryl substrate comprises fused aryl rings, the departing substituent is bonded to the aryl ring carbon (C¹) through an oxygen atom. In some embodiments, when the ortho-directing group is ester and the aryl substrate is a phenyl ring, the departing substituent is bonded to an aryl ring carbon (C¹) through a nitrogen atom.

In a second aspect the invention provides a method of removing a NR₂ or OR substituent from an aromatic substrate, comprising combining in an inert atmosphere to form a reaction mixture: (i) an aromatic substrate that comprises a ring carbon substituted by NR₂ or OR, wherein said NR₂ or OR is located ortho to an ortho-directing group; (ii) a reductant; and (iii) a catalytic amount of a ruthenium or rhodium complex; allowing reaction to proceed under suitable conditions of temperature and pressure for an appropriate reaction time to produce a product that is a modified form of the aromatic substrate, wherein the modification is that the NR₂ or OR substituent has been replaced by H; wherein R is aliphatic, aryl, aliphatic-aryl or aryl-aliphatic.

In embodiments of this aspect the reaction mixture is neat. In certain embodiments of this aspect the reaction mixture comprises solvent. In embodiments of this aspect the reductant is Et₃SiH or DIBAL-H. In certain embodiments of this aspect the reaction is hydrodemethoxylation of a biaryl amide and the reductant is Et₃SiH. In embodiments of this aspect, the reaction is hydrodemethoxylation of a naphthamide and the reductant is Et₃SiH. In certain embodiments of this aspect the reaction is hydrodemethoxylation of a benzamide and the reductant is DIBAL-H. In some embodiments of this aspect, the ruthenium or rhodium complex comprises RuH₂(CO)(PPh₃)₃, Ru₃(CO)₁₂, Ru(CO)₂(PPh₃)₃, Cp*Rh(C₂H₃SiMe₃)₂, or RuHCl(CO)(PPh₃)₃. In certain embodiments, the ruthenium complex comprises RuH₂(CO)(PPh₃)₃. In embodiments of this aspect, the ortho-directing group is an amide moiety. In certain embodiments of this aspect, amide moiety is C(O)NEt₂, or C(O)NMe₂. In some embodiments of this aspect, combining in an inert atmosphere comprises mixing in a N₂ or argon atmosphere, or mixing in a tube under N₂ or argon and then sealing the tube. An embodiment of this aspect further comprises filtering through silica gel to separate any solids, reducing the volume of filtrate under vacuum, and purifying.

In a third aspect the invention provides a compound which is:

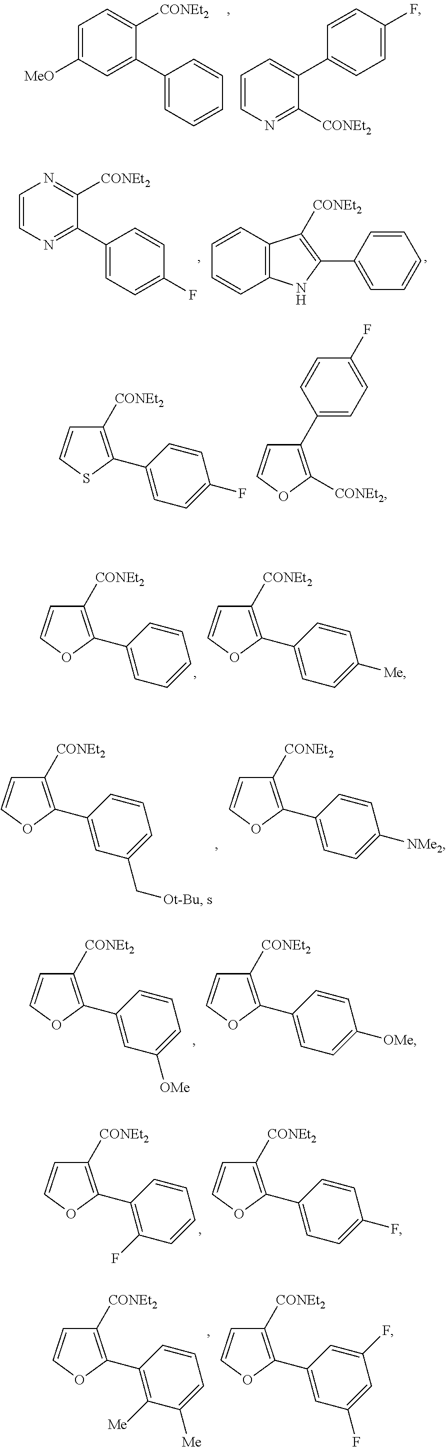

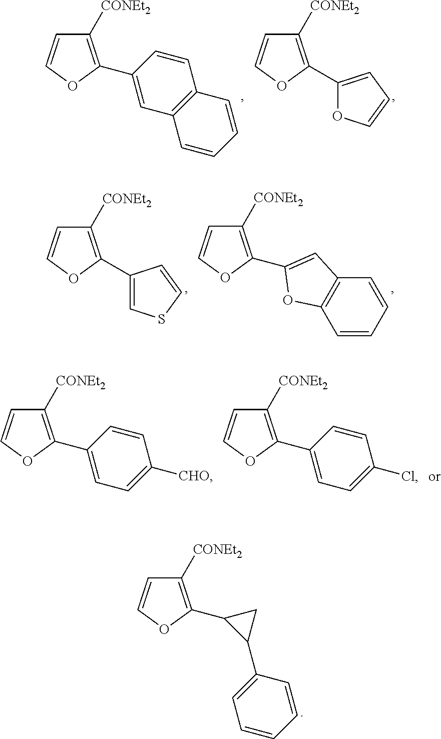

In a fourth aspect the invention provides a compound which is:

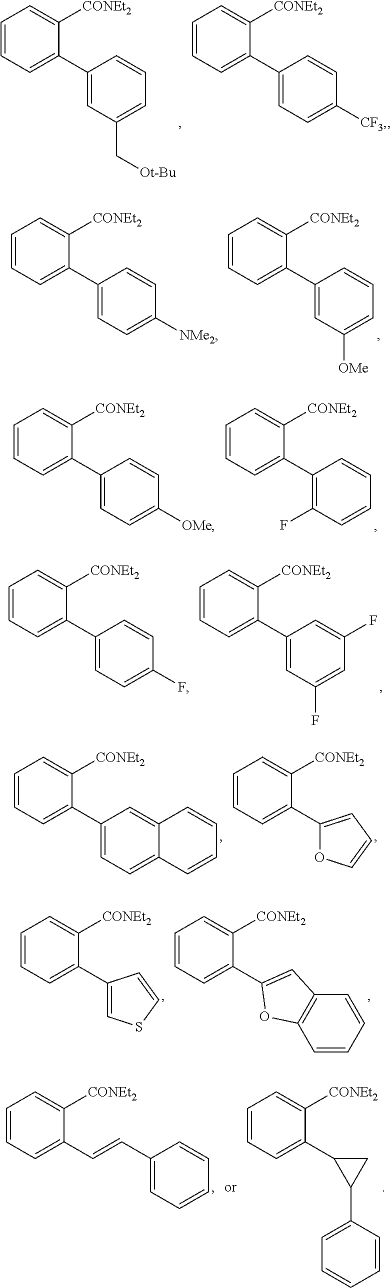

In a fifth aspect the invention provides a compound which is:

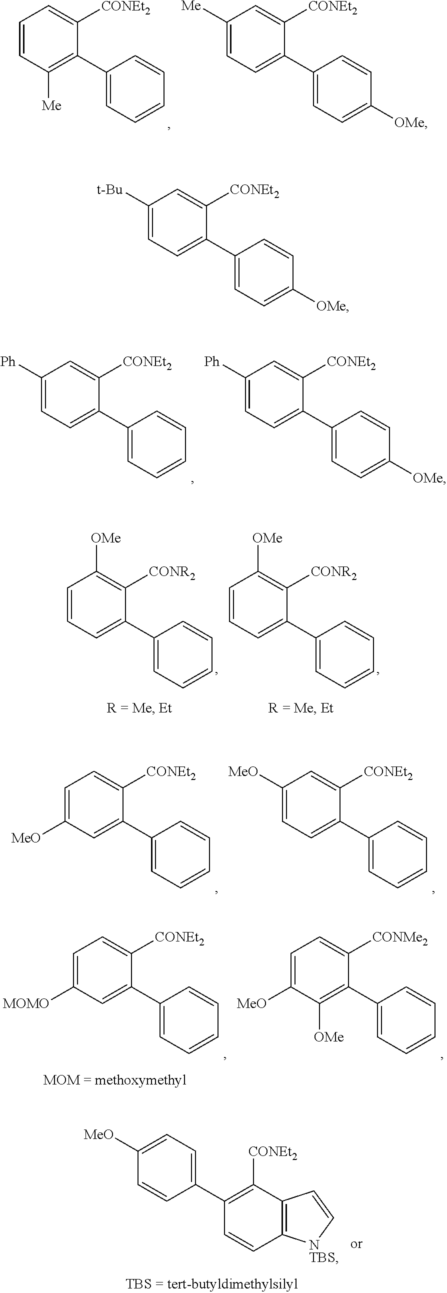

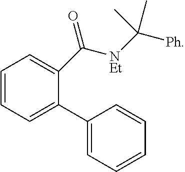

In a sixth aspect the invention provides a compound which is:

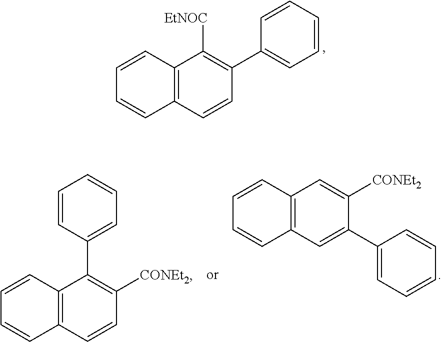

In a seventh aspect the invention provides a compound which is:

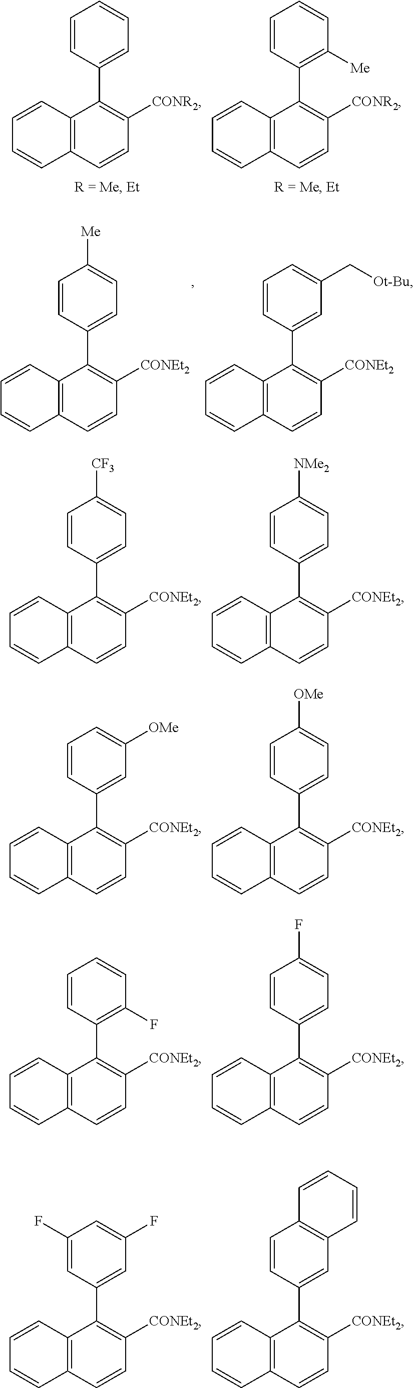

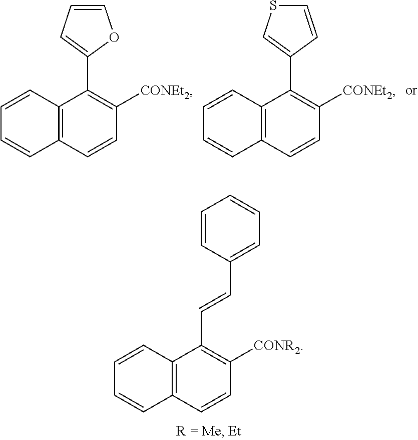

In an eighth aspect the invention provides a compound which is:

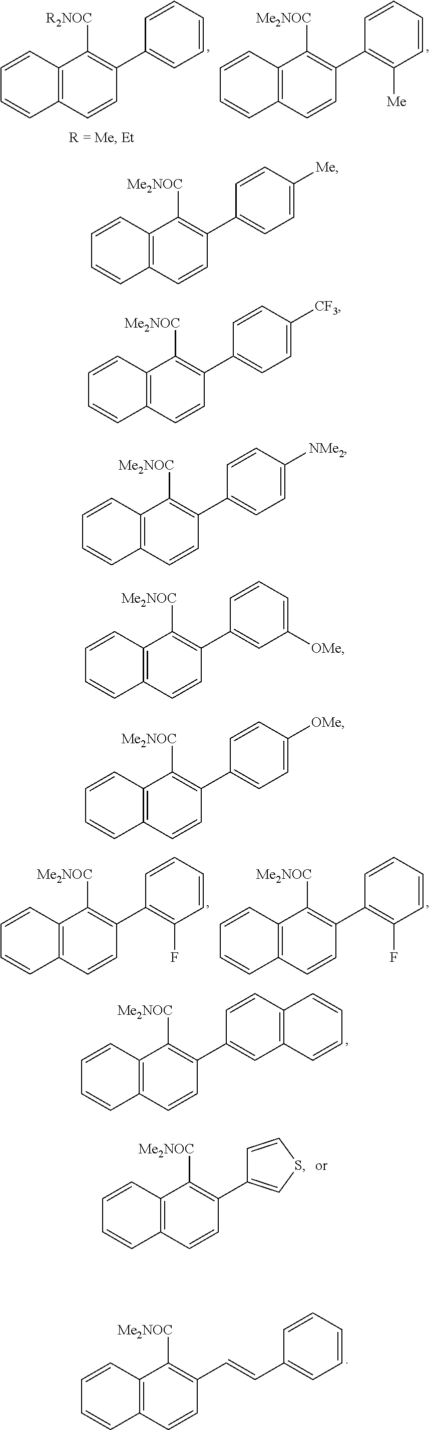

In a ninth aspect the invention provides a compound which is:

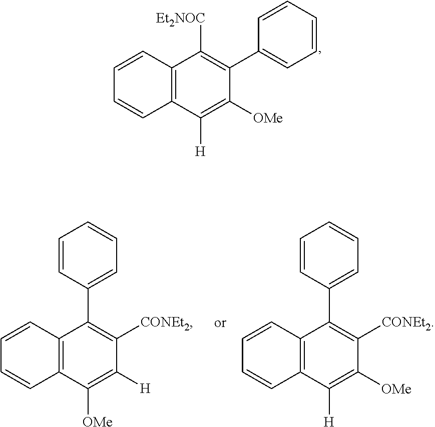

In a tenth aspect the invention provides a compound which is:

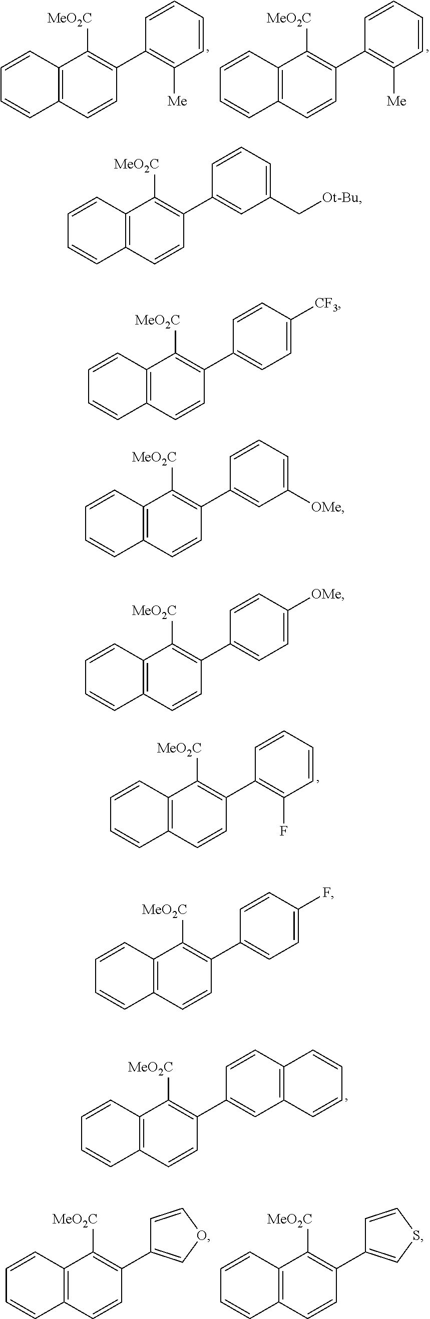

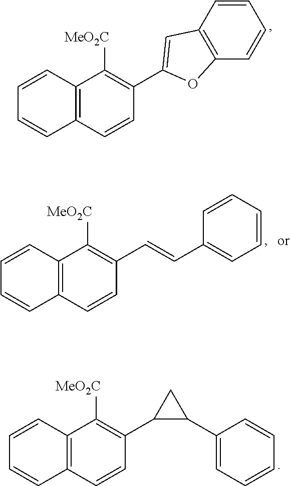 [SMILES: COC(=O)c1c(-c2cc3ccccc3o2)ccc2ccccc12 ; COC(=O)c1c(/C=C/c2ccccc2)ccc2ccccc12 ; COC(=O)c1c(C2CC2c2ccccc2)ccc2ccccc12]

In an eleventh aspect the invention provides a method of making a compound of Table 2, comprising combining in an appropriate solvent and under an inert atmosphere to form a reaction mixture: an aryl substrate bearing a tertiary amide ortho-directing group ortho to a hydrogen; a boronate comprising a boron bonded through a carbon atom to an addition moiety; and a catalytic amount of a ruthenium or rhodium complex; allowing reaction to proceed under suitable conditions of temperature and pressure for an appropriate reaction time to produce a product that is a modified form of the aryl substrate, wherein the modification is that the addition moiety has replaced the hydrogen.

In embodiments of this aspect the amide is CONEt₂. In certain embodiments of this aspect the appropriate solvent is toluene. In some embodiments of this aspect, the ruthenium or rhodium complex comprises RuH₂(CO)(PPh₃)₃, Ru₃(CO)₁₂, Ru(CO)₂(PPh₃)₃, Cp*Rh(C₂H₃SiMe₃)₂, or RuHCl(CO)(PPh₃)₃. In certain embodiments, the ruthenium complex comprises RuH₂(CO)(PPh₃)₃. In certain embodiments of this aspect, the suitable conditions of temperature comprises heating to 120° C. In certain embodiments of this aspect, the addition moiety is aliphatic, aryl, or a combination thereof. In some embodiments of this aspect, the appropriate reaction time is about 24 h to 44 h. In certain embodiments of this aspect, the boronate is added in excess relative to the substrate.

In a twelfth aspect the invention provides a method of making a compound of Table 3, comprising combining in an appropriate solvent and under an inert atmosphere to form a reaction mixture: an aryl substrate bearing an amide directing group ortho to an NR₂ moiety, a boronate comprising a boron bonded through a carbon atom to an addition moiety; and a catalytic amount of a ruthenium or rhodium complex; allowing reaction to proceed under suitable conditions of temperature and pressure for an appropriate reaction time to produce a product that is a modified form of the aryl substrate, wherein the modification is that the addition moiety has replaced the NR₂ moiety.

In certain embodiments of this aspect, the amide is CONEt₂. In some embodiments of this aspect, the appropriate solvent is toluene. In certain embodiments of this aspect, the ruthenium or rhodium complex comprises RuH₂(CO)(PPh₃)₃, Ru₃(CO)₁₂, Ru(CO)₂(PPh₃)₃, Cp*Rh(C₂H₃SiMe₃)₂, or RuHCl(CO)(PPh₃)₃. In certain embodiments, the ruthenium complex comprises RuH₂(CO)(PPh₃)₃. In some embodiments of this aspect, the suitable conditions of temperature comprises heating to 125° C. In some embodiments of this aspect, the addition moiety is aliphatic, aryl, or a combination thereof. In embodiments of this aspect, the appropriate reaction time is about 1 h to 20 h. In some embodiments of this aspect, the boronate is added in excess relative to the substrate.

In a thirteenth aspect, the invention provides a method of making a compound of Table 5, comprising combining in an appropriate solvent and under an inert atmosphere to form a reaction mixture: an aryl substrate bearing an amide directing group ortho to an alkoxy moiety, a boronate comprising a boron bonded through a carbon atom to an addition moiety; and a catalytic amount of a ruthenium or rhodium complex; allowing reaction to proceed under suitable conditions of temperature and pressure for an appropriate reaction time to produce a product that is a modified form of the aryl substrate, wherein the modification is that the addition moiety has replaced the alkoxy moiety.

In some embodiments of this aspect, the amide is CONEt₂. In some embodiments of this aspect, the appropriate solvent is toluene. In some embodiments of this aspect, the ruthenium or rhodium complex comprises RuH₂(CO)(PPh₃)₃, Ru₃(CO)₁₂, Ru(CO)₂(PPh₃)₃, Cp*Rh(C₂H₃SiMe₃)₂, or RuHCl(CO)(PPh₃)₃. In certain embodiments, the ruthenium complex comprises RuH₂(CO)(PPh₃)₃. In some embodiments of this aspect, the suitable conditions of temperature comprises heating to 125° C. In certain embodiments of this aspect, the addition moiety is aliphatic, aryl, or a combination thereof. In some embodiments of this aspect, the appropriate reaction time is about 20 h. In some embodiments of this aspect, the boronate is added in excess relative to the substrate.

In a fourteenth aspect the invention provides a method of making a compound of Table 6, comprising combining in an appropriate solvent and under an inert atmosphere to form a reaction mixture: an aryl substrate bearing an amide directing group ortho to an alkoxy moiety and at least one other substitutent, a boronate comprising a boron bonded through a carbon atom to an addition moiety; and a catalytic amount of a ruthenium or rhodium complex; allowing reaction to proceed under suitable conditions of temperature and pressure for an appropriate reaction time to produce a product that is a modified form of the aryl substrate, wherein the modification is that the addition moiety has replaced the alkoxy moiety.

In certain embodiments of this aspect, the amide is CONEt₂. In certain embodiments of this aspect, the appropriate solvent is toluene. the ruthenium or rhodium complex comprises RuH₂(CO)(PPh₃)₃, Ru₃(CO)₁₂, Ru(CO)₂(PPh₃)₃, Cp*Rh(C₂H₃SiMe₃)₂, or RuHCl(CO)(PPh₃)₃. In certain embodiments, the ruthenium complex comprises RuH₂(CO)(PPh₃)₃. In certain embodiments of this aspect, the suitable conditions of temperature comprises heating to 125° C. In some embodiments of this aspect, the addition moiety is an aryl moiety with a substituent para to the boron. In certain embodiments of this aspect, the addition moiety is aliphatic, aryl, or a combination thereof. In embodiments of this aspect, the appropriate reaction time is about 20 h. In certain embodiments of this aspect, the boronate is added in excess relative to the substrate.

In a fifteenth aspect the invention provides a method of forming an aryl ring that is at least di-substituted, comprising (a) combining in an inert atmosphere to form a reaction mixture: (i) an aryl substrate that has a substituent that is an amide ortho-directing group in an ortho position to a departing substituent, wherein the departing substituent is bonded to a ring carbon of the aryl substrate through a hydrogen, oxygen, or nitrogen atom, (ii) a boronate comprising a boron bonded to an addition moiety through a carbon; and (iii) a catalytic amount of a ruthenium or rhodium complex; (b) allowing reaction to proceed under suitable conditions of temperature and pressure for an appropriate reaction time to produce a cross coupling product that is a modified form of the aryl substrate, wherein the modification is that the addition moiety has replaced the departing substituent and is bonded through its carbon to the aryl ring carbon, and is ortho to the directing group; (d) combining to form a mixture (iv) Cp₂ZrCl₂, (v) a reducing agent LiAlH(OBu-t)₃, LiBH(s-Bu)₃, or a combination thereof, and (vi) the cross coupling product of step (b) wherein (iv) and (v) react to produce an intermediate product, which intermediate product then reacts with the cross coupling product to form a reduction product that is a reduced form of the cross coupling product.

In certain embodiments of this aspect, the cross coupling product is an amide-substituted aryl compound. In certain embodiments of this aspect, the reduction product is an aldehyde-substituted aryl compound.

In a sixteenth aspect the invention provides a compound made by the method of the fifteenth aspect. In embodiments of the sixteenth aspect, the cross coupling product is a compound of the third to ninth aspects. In certain embodiments of this aspect, the reduction product is an aryl compound bearing an aldehyde moiety in place of the cross coupling product's amide moiety. In an embodiment of the sixteenth aspect, the compound is:

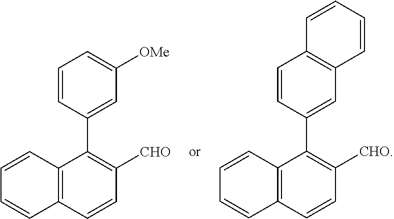 [SMILES: COc1cccc(-c2c(C=O)ccc3ccccc23)c1 ; O=Cc1ccc2ccccc2c1-c1ccc2ccccc2c1]

In a seventeenth aspect the invention provides a compound comprising an aryl ring substituted by an amide and an aliphatic, aryl, aliphatic-aryl, or aryl-aliphatic substituent in an ortho position relative to the amide.

In a eighteenth aspect the invention provides a compound comprising an aryl ring substituted by an ester and an aliphatic, aryl, aliphatic-aryl, or aryl-aliphatic substituent in an ortho position relative to the ester.

In a nineteenth aspect the invention provides a compound made by the method of the fifteenth aspect comprising an aryl ring substituted by an amide and a H-substituent in the ortho position.

In embodiments of the seventeenth to nineteenth aspects, the invention provides a compound comprising further substituents.

In an twentieth aspect, the invention provides a compound which is:

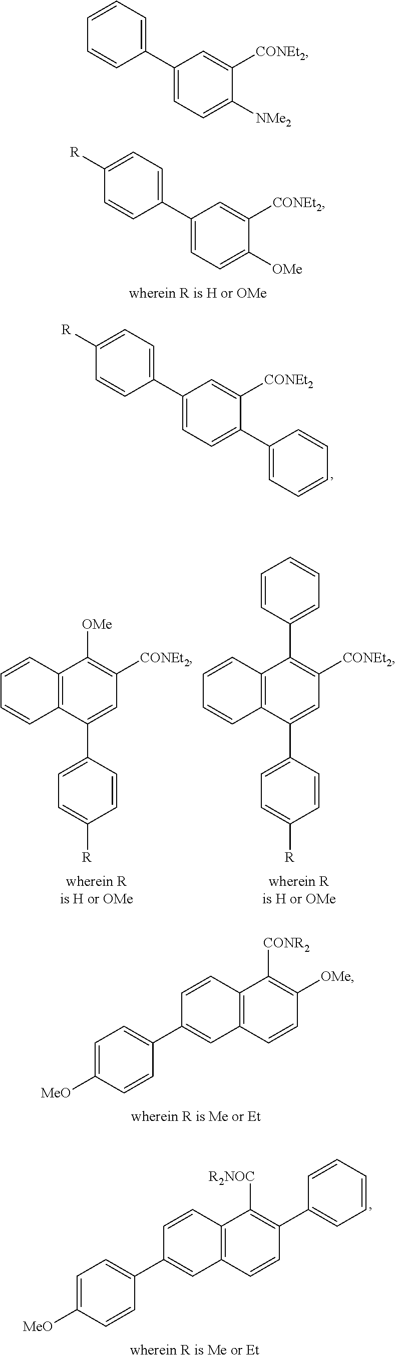

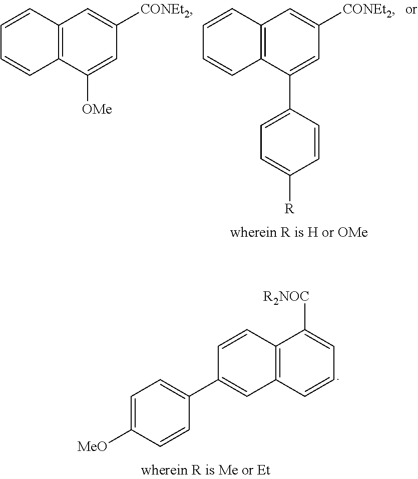

Other objects and advantages of the present invention will become apparent from the disclosure herein.

### BRIEF DESCRIPTION OF THE DRAWINGS

For a better understanding of the invention and to show more clearly how it may be carried into effect, reference will now be made by way of example to the accompanying drawings, which illustrate aspects and features according to embodiments of the present invention, and in which:

FIG. 1 shows Schemes 1, 2, 3 and 4. Scheme 1 shows a DoM-Suzuki coupling strategy. Scheme 2 shows initial test strategies for amide-directed C—N and C—O activation/coupling reactions. Scheme 3 depicts a synthesis of teraryls via a Bromination-Suzuki Coupling-C—O Activation/Coupling Sequence. Scheme 4 Bromination-Suzuki coupling-C—N activation/coupling sequence.

FIG. 2 shows methods for preparation of substituted ortho-anisamides.

FIG. 3 shows Scheme 5, which depicts a synthesis of teraryls via sequential bromination, standard Suzuki Cross Coupling and C—O Activation/Coupling Reactions.

FIG. 4 shows Scheme 6, which depicts a synthesis of naphthyl-based biaryls via a bromination-Suzuki Cross Coupling-hydrodemethoxylation sequence.

FIG. 5 shows Scheme 7, which presents ideas for uses of compounds described herein.

### DETAILED DESCRIPTION OF PREFERRED EMBODIMENTS

### Definitions

As used herein, the term “cross coupling” refers to a type of chemical reaction where two hydrocarbon fragments are coupled together with aid of a metal containing catalyst.

As used herein, the term “DG” or “directing group” refers to a substituent on an aryl ring that directs an incoming electrophile to a specific relative position (e.g., ortho, meta, para).

As used herein, the term “hydrodemethoxylation” refers to a process wherein a methoxy (MeO) substituent on an aryl ring is replaced by a H.

As used herein, the term “activating group” refers to a functional group when an aryl ring, to which it is attached, more readily participates in electrophilic substitution reactions. Activating groups are generally ortho/para directing for electrophilic aromatic substitution.

As used herein, the term “aliphatic” refers to hydrocarbon moieties that are straight chain, branched or cyclic, may be alkyl, alkenyl or alkynyl, and may be substituted or unsubstituted.

As used herein, the terms “short chain aliphatic” or “lower aliphatic” refer to C₁ to C₄ aliphatic; the terms “long chain aliphatic” or “higher aliphatic” refer to C₅ to C₂₅ aliphatic.

As used herein, “heteroatom” refers to non-hydrogen and non-carbon atoms, such as, for example, O, S, and N.

As used herein, “Boc” refers to tert-butoxycarbonyl. As used herein, “Cbz” refers to benzyloxycarbonyl. As used herein, “TMS” refers to trimethylsilyl. As used herein, “Tf” refers to trifluoromethanesulfonyl.

As used herein, the term “aryl” means aromatic, including heteroaromatic.

As used herein, the term “amide” means a moiety including a nitrogen where at least one of the groups bound to the nitrogen is an acyl (i.e., —C(═O)—) group.

As used herein, the term “reduction” or “reduce” refers to a reaction that converts a functional group from a higher oxidation level to a lower oxidation level. Typically, a reduction reaction either adds hydrogen or removes an electronegative element (e.g., oxygen, nitrogen, or halogen) from a molecule.

As used herein, the term “benzamide” refers to a compound with a phenyl aryl group that has a —C(═O)NR^{b}R^{c} group bound to one of its ring atoms, where R^{b} and/or R^{c} may be hydrogen, substituted or unsubstituted lower aliphatic, and substituted or unsubstituted higher aliphatic.

As used herein, the term “Georg method” refers to a method of using pre-prepared Schwartz Reagent as a reducing agent that specifically targets certain functional groups, as described in White, J. M., Tunoori, A. R., Georg, G. I., J. Am. Chem. Soc. 2000, 122, 11995-11996.

As used herein, the term “tertiary amide” means a moiety including a nitrogen that is bonded to a carbonyl group where the nitrogen is also bonded to non-hydrogen moieties, i.e., R^{a}C(═O)NR^{d}R^{e} where R^{d} and/or R^{e} are typically aliphatic, but are not hydrogen. This should not be confused with a lesser-known use of the term “tertiary amide”; specifically, where there are three acyl groups on an amide nitrogen, i.e., [R^{a}C(═O)]₃N (this latter use is discussed in IUPAC Compendium of Chemical Terminology, 2^{nd} ed. (1997) by Alan D. McNaught and Andrew Wilkinson, Royal Society of Chemistry, Cambridge, UK).

As used herein, the term “LiAlH(OBu-t)₃” means lithium tri-(tert-butoxy)aluminum hydride, and the term “LiBH(s-Bu)₃” means lithium tri-(sec-butyl)borohydride.

As used herein, the term “DIBAL-H” means diisobutylaluminum hydride.

As used herein, the term “Schwartz Reagent” means bis(cyclopentadienyl)-zirconium(IV) chloride hydride, which is also referred to herein as Cp₂Zr(H)Cl.

As used herein, Schwartz Reagent Precursor means bis(cyclopentadienyl)-zirconium(IV) dichloride (Cp₂ZrCl₂).

As used herein, the term “in situ” has its ordinary chemical meaning of presence of a molecule in a reaction where it is generated therein instead of separately added.

As used herein, the term “substrate” means a compound that is desired to be converted to a product compound.

As used herein, the term “suitable conditions of temperature and pressure” means applying sufficient heat and/or pressure for a reaction to proceed. As one of skill in the art will know, under atmospheric pressure more heat may be required for a reaction to proceed than under higher presssure conditions.

### Embodiments

Methods are described herein for eliminating a substituent of an aryl substrate that is in an ortho prosition to an amide or ester directing group. Methods are also provided to form a C—C bond (i.e., cross coupling) between aryl substrates and aryl and/or aliphatic substituents whereby the substituents bond at an ortho position relative to an ester or amide directing group. Further methods are provided to convert aryl amides to aryl aldehydes. Many compounds have been prepared using such methods. Syntheses and characterization data for these compounds is also provided herein.

In contrast to most cross coupling reactions, these processes allow minimization of potentially damaging waste products. Starting materials are commercially available or easily prepared from inexpensive chemicals and the large number of new products of the reaction that have been prepared can be easily transformed to useful building blocks for organic syntheses by chemists working in pharmaceutical and material science areas. Furthermore, these methods exhibit potential for application in multi-step commercial synthesis.

Ruthenium and rhodium complexes as described herein include RuH₂(CO)(PPh₃)₃, Ru₃(CO)₁₂, Ru(CO)₂(PPh₃)₃, Cp*Rh(C₂H₃SiMe₃)₂, and RuHCl(CO)(PPh₃)₃, where Cp* is pentamethylcyclopentadiene.

For simplicity, the methods described herein are described according to the type of bond that is activated (e.g., C—H, C—O, C—N).

### C—H Activation of Aryl-Amide and Heteroaryl-Amide and C—C Bond Formation

A catalytic amide-directed C—H activation/C—C bond forming process for aryl-amide including heteroaryl-amide was tested. Arylation of O-heteroaryl amides, N-heteroaryl amides and S-heteroaryl amides was obtained in good to excellent yields as shown in Table 2. In contrast, ketone-directed C—H activation/arylation of furan systems has not been reported. A variety of arylboronates having electron donating substituents such as Me, CH₂Ot-Bu, NMe₂ and OMe were employed and high yields were obtained. Similarly, arylboronates having electron withdrawing substituents such as F and CF₃, underwent arylation in good yields.

Notably, amide reduction was not observed under the cross coupling conditions used. This suggests that in contrast to the ketone-directed C—H activation/arylation reaction in which pinacolone solvent or 2 equivalents of a ketone substrate were required to act as hydride scavengers for Ru—H species to maintain the catalytic cycle, the Ru—H cannot effect reduction of the amide group. Thus, necessity for using pinacolone (or acetone) as solvent is eliminated and alternative solvents (e.g., toluene) may be used.

### C—N Activation and C—C Bond Formation

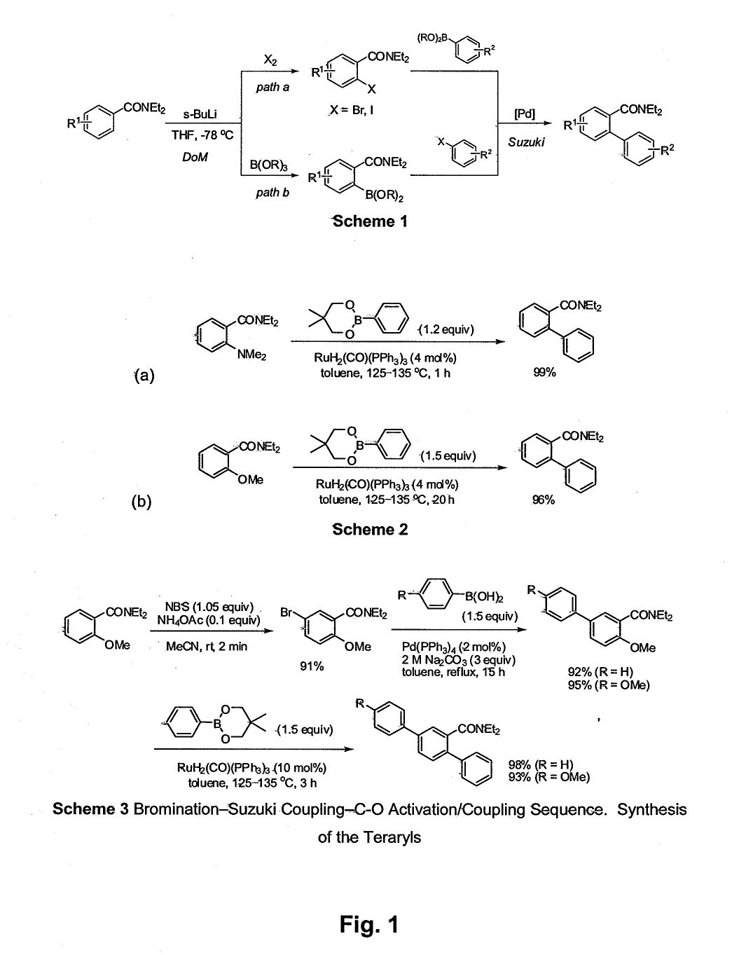

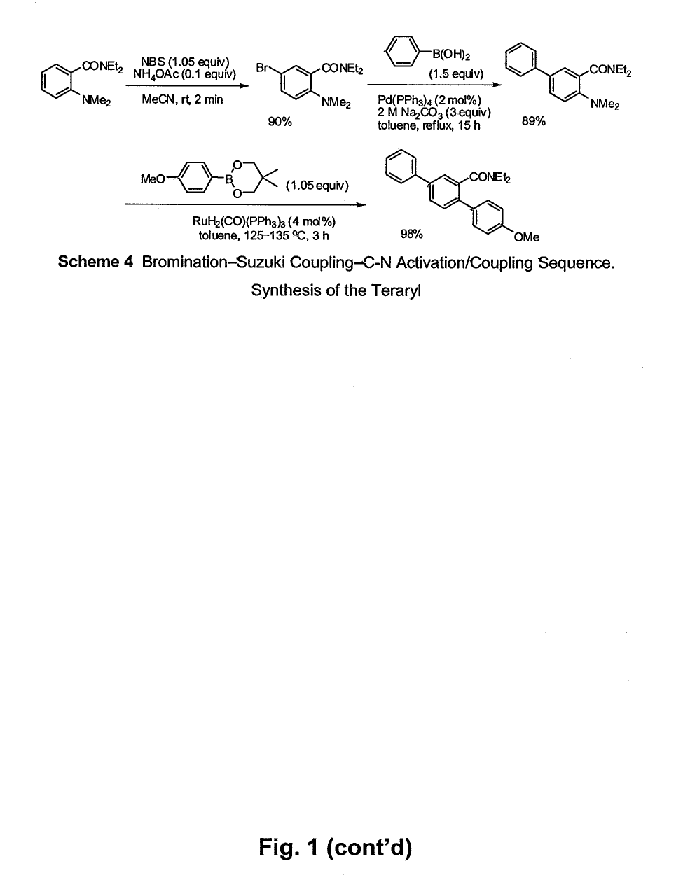

Catalytic C—N activation/C—C bond formation is an exciting area of chemistry. To date, the Ru-catalyzed ketone-directed C—N activation/C—C bond forming reaction was reported by Kakiuchi and co-workers as a part of the study of chelation-assisted reactions of aromatic ketones with organoboronates. A catalytic amide-directed C—N activation/C—C bond forming process was tested under RuH₂(CO)(PPh₃)₃/toluene conditions. Treatment of 2-Me₂N—N,N-diethylbenzamide with phenyl boroneopentylate led in 1 h to the formation the diphenyl amide in almost quantitative yield with no observation of the alternative C—H activation/arylation product (see Scheme 2a, FIG. 1).

Following successful demonstration of the amide-directed C—N activation/coupling reaction for 2-Me₂N—N,N-diethyl benzamide, the generality of the reaction was tested with a variety of aryl boroneopentylates and results are shown in Table 3, Cross Coupling Reactions of 2-Me₂N—N,N-diethyl Benzamide with Aryl Boroneopentylates.

To demonstrate the methods described herein in a example synthesis, a methodology and convenient sequence was developed wherein a substituted triaryl compound was produced in excellent overall yield. As shown in Scheme 4, FIG. 1, a first reaction step was bromination using NBS (N-Bromosuccinimide), the second reaction step was a standard Suzuki C—C cross coupling, and the final reaction step was a C—N activation/cross coupling reaction. Conversion into the bromobenzamide was achieved in high yield. The subsequent standard Suzuki cross coupling gave the biaryl product which, upon C—N activated coupling with the anisyl boroneopentylate afforded the teraryl in 80% overall yield in three steps. Notably, the C—N activation/coupling step proceeded in almost quantitative yield.

### Amide-Directed C—O Bond Activation/Cross Coupling

Amide-directed catalytic C—O activation/cross coupling reactions carried out under simple RuH₂(CO)(PPh₃)₃/solvent conditions were investigated (see Scheme 2b, FIG. 1). Notably, C—H activation byproducts were not observed. These results show that tertiary amide directing groups activate C—O bonds. Such directing groups may exhibit greater coordination ability than ketone. In addition, these results indicate that CONEt₂ behaved similarly to the t-butyl group of ortho-methoxyphenyl t-butyl ketone in preventing non-regioselective C—H and C—O activation and therefore diarylation. Results of coupling of ortho-anisamides with a variety of aryl boroneopentylates including substituted aryl boronates are summarized in Tables 5 and 6. Importantly, this amide-directed catalytic arylation reaction provided a catalytic base-free DoM-cross coupling process at non-cryogenic temperatures.

This method has several advantages over the Kakiuchi ketone-directed C—O activation/coupling reaction: i) it is not compromised by a C—H activation cross coupling reaction; and ii) compared to the intractable t-butyl ketone products, the resulting amides are potentially useful in further amide-related chemistry. The corresponding C—O activated coupling reaction is a significant advance of the Ru-catalyzed ketone-directed C—O activation/coupling reaction developed by Murai, Kakiuchi, and co-workers.

As an application of the above methodology, a Ru-catalyzed C—O and normal Suzuki cross coupling sequence was developed for the synthesis of teraryls (see Scheme 3, FIG. 1). The overall synthesis combines classical electrophilic substitution and two catalytic cross coupling reactions in an overall efficient synthesis of teraryls (80% overall yield in 3 steps).

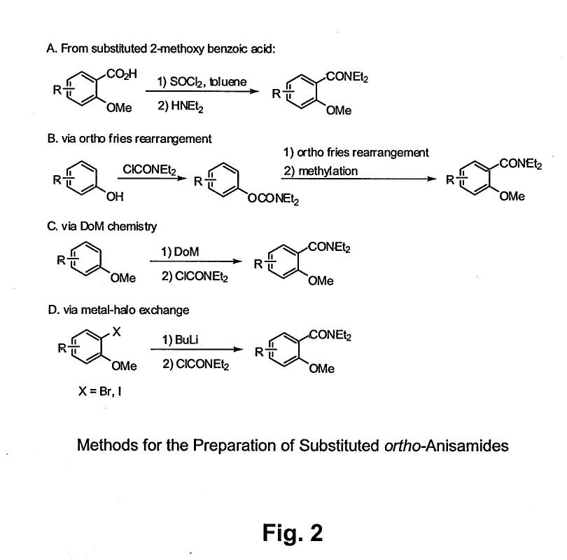

Based on the results described above, a general and efficient amide-directed C—O activation/cross coupling methodology for the synthesis of biaryl and heterobiaryl amides has been developed. This methodology has high practical value in that, compared to the preparation of starting materials for the Kakiuchi ketone-directed coupling reaction, the substituted ortho-anisamides are readily available from simple and inexpensive commodity chemicals. Four common methods (A-D) of preparation are shown in FIG. 2. Method A starts from substituted salicylic ortho-anisic acids to afford the corresponding amides in classical two-step one-pot sequence. Method B involves the anionic ortho Fries rearrangement (Ma, Y. et al., J. Am. Chem. Soc. 2007, 129, 14818-14825; Singh, K. J. et al., J. Am. Chem. Soc. 2006, 128, 13753-13760; Tsukazaki, M. et al., Can. J. Chem. 1992, 70, 1486-91) to lead to ortho-anisamides in three simple steps starting from commercially available phenols. This method also has the benefit of DoM chemistry to obtain unusually substituted derivatives. Method C starts from substituted anisoles to afford the 2-MeO benzamides in a single step via DoM chemistry. Method D shows a process to form 2-MeO benzamides via metal-halogen exchange. The facile and multiple routes for the preparation of 2-MeO benzamides will make the amide-directed C—O activation/coupling reaction of practical interest.

In summary, we have demonstrated the first catalytic amide-directed C—O activation/C—C cross coupling reaction. The reaction is efficient, highly regiospecific and has considerable practical potential. The catalytic reaction may be viewed as complementing or superceding the DoM-cross coupling strategy (Anctil, E. J. G. et al., Metal-Catalyzed Cross-Coupling Reactions (2nd Ed) 2004, 2, 761-813, Wiley: Weinheim; Anctil, E. J. G. et al., J. Organomet. Chem. 2002, 653, 150-160; Green, L. et al., J. Heterocycl. Chem. 1999, 36, 1453-1468.) with advantage of non-cryogenic temperatures and non-requirement of base.

### C—O Activation of Naphthamides and C—C Bond Formation

As demonstrated above, ortho-anisamides are highly reactive partners for Ru-catalyzed amide-directed C—O activation/C—C cross coupling reaction with aryl boroneopentylates (see Tables 8 and 9). Further studies explored whether similar success could be found for naphthamides. Several ortho-MeO naphthamides were studied and initial results are presented in Table 7. Yields of cross coupling products varied as a function of methoxy naphthamide isomers. Thus 2-MeO-1-naphthamide and 1-MeO-2-naphthamide underwent C—O activation/cross coupling reactions to afford the biaryl products in excellent yields while the 3-MeO-2-naphthamide gave product in much lower yield. As also observed for the C—O cross coupling reactions of benzamides (Tables 5 and 6), no C—H activation/coupling products were formed. To note again, in contrast to the Kakiuchi ketone-directed C—O activation/cross coupling reaction, the corresponding naphthamide coupling reaction is for ortho C—O activation and is inert to the ortho C—H bond activation process.

These initial results motivated an investigation of the generality of the reaction with a variety of organoboronates and results are shown in Table 8. These results establish a general, efficient, and potentially useful route for the preparation of 1-arylated naphthalenes. As indicated by the observed high yields in all reactions, no peri-hindrance effect inhibits the C—O activated coupling (Kumar, D. et al. Synthesis 2008, 1249-1256; Lakshmi, A. et al., J. Phys. Chem. 1978, 82, 1091-1095).

Generality of the reaction of 2-MeO-1-naphthamides was then investigated with a variety of organoboronates. Considering a possible steric conflict between the peri-hydrogen and an amide group, 2-MeO-N,N-dimethyl-1-naphthamide was employed to minimize problems of peri steric hindrance in coupling with ortho-substituted aryl boroneopentylates. Results are shown in Table 9. Based on these results, this method may provide a useful route for making 2-arylated naphthalenes.

Having completed a study concerning scope of aryl boroneopentylates in the cross coupling reaction, we investigated the scope of naphthamide coupling partners and results are presented in Table 10. Entries 1 and 2 demonstrate that selective ortho to amide C—O bond activation/cross coupling occurs to give the ortho-phenylated products in quantitative yields, which reinforces the significance of amide directing and chelation assistance in the reaction. Interestingly, entry 3 shows that, in the presence of C-1 and C-3 C—O bonds, C-1 C—O activation/cross coupling selectivity is observed. This result confirms the higher C-1 compared to the C-3 C—O activation reactivity, which was also observed in studies of other isomeric methoxy naphthamides (see Table 7).

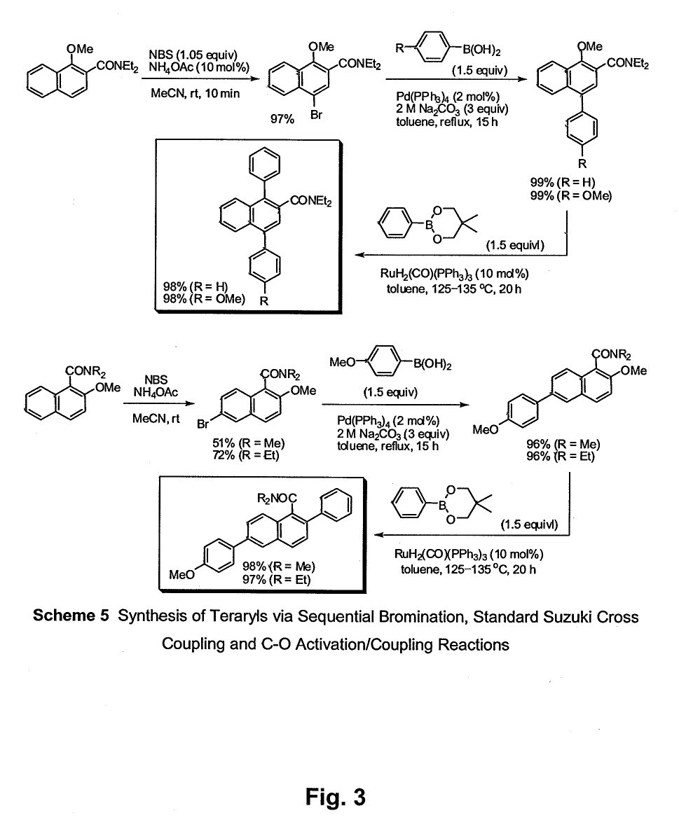

Analogous to the previous study, combined C—O and standard Suzuki cross coupling tactics (Scheme 3) were investigated, a similar high yield process was developed which involved bromination, Suzuki coupling and the C—O activation/coupling for the construction of teraryls incorporating a functionalized central naphthalene ring (Scheme 5, FIG. 3).

In summary, an efficient and highly regioselective Ru-catalyzed naphthamide coupling methodology has been established that constitutes a first catalytic amide-directed C—O activation of naphthamides/C—C bond cross coupling reaction. It complements and may supercede the DoM Suzuki cross coupling strategy since it has the advantages of non-cryogenic and base-free conditions. In addition, it provides naphthamides which are difficult to prepare by the traditional DoM-Suzuki cross coupling sequence (see Table 8).

### Ester-Directed C—O Activation and C—C Bond Formation

The first Ru-catalyzed ester-directed C—H activation/arylation was reported by Kakiuchi and co-workers (Kitazawa, K. et al. J. Organomet. Chem. 2010, 695, 1163-1167). The disadvantage of this method is that the formation of a mixture of mono- and di-arylated products cannot be avoided even when the required isopropyl ester is used as the directing group.

An ester-directed C—O activation/arylation reaction has not been reported to date. Several experiments were formulated to test whether ester may have the appropriate directing features for C—O activation/C—C bond formating reaction, results are shown in Table 11. Commercially available ortho-anisic ester led to only trace amounts of C—O activation/cross coupling product (Table 11, entry 1). However, of the three regioisomeric naphthoates, 2-MeO-1-naphthoate showed excellent reactivity for a C—O activation/phenylation reaction while the isomeric 1-methoxy ester was modestly reactive and the 3-methoxy ester was unreactive (Table 11, entries 2-4). Accordingly, these studies constitute the first examples of ester-directed C—O activation/cross coupling reaction.

The recognition that the 2-MeO-1-naphthoate ester has an excellent reactivity and selectivity for a C—O activation/phenylation reaction stimulated a study concerning the generalization of the reaction for a variety of aryl boroneopentylates and the results are shown in Table 12.

In summary, a highly efficient and regioselective Ru-catalyzed naphthoate ester-directed C—O activation/cross coupling methodology has been discovered and generalized. Together with the benzoate results, it constitutes a new reaction which extends the Murai, Kakiuchi chemistry from ketone- to ester-directed reactions.

This method is the first catalytic ester-directed C—O activation/C—C bond formation reaction. It proceeds with high efficiency and regioselectivity and may be viewed as a complement and perhaps a replacement of the DoM-Suzuki cross coupling strategy with advantages of non-cryogenic temperatures and base-free conditions. This reaction has the potential to become a most highly efficient and practical cross coupling route for preparation of 2-aryl and heteroaryl naphthoate acids and esters from easily available 1-naphthoate ester derivatives.

### C—O Activation and Reduction (Hydrodemethoxylation)

The current popular method for reductive removal of a phenol or alkoxy substituent from an aromatic substrate is via conversion to a C—OTf derivative and catalytic hydrodetriflation (Cacchi, S. et al., Tetrahedron Lett. 1986, 27, 5541-5544; Peterson, G. A. et al., Tetrahedron Lett. 1987, 28, 1381-1384; Saa, J. M. et al., J. Org. Chem. 1990, 55, 991-995; Behenna, D. C. et al., Angew. Chem. Int. Ed. 2007, 46, 4077-4080; and Hupp, C. D. et al., Tetrahedron Lett. 2010, 51, 2359-2361). The requirement of preparation of the triflate using expensive triflic anhydride or PhNTf₂ represents a major limitation of this procedure. An available direct hydrodemethoxylation of aromatic C—OMe derivatives via a catalytic C—O cleavage would constitute a useful contribution to organic synthesis.

Based on the above studies of amide-directed C—O activation/cross coupling reactions, it was considered that a hydrodemethoxylation reaction of aromatic OMe derivatives may be achieved via a C—O activation/reduction by a hydride source. An absolute requirement for the success of the process was that the chosen hydride reagent not reduce the amide group.

Initially, several reductants were tested for the hydrodemethoxylation reaction of 1-MeO-N,N-diethyl-2-naphthamide using RuH₂(CO)(PPh₃)₃ catalysis and the results are tabulated in Table 13. Using Et₃SiH afforded the hydrodemethoxylation product in almost quantitative yield (Table 13, entry 1) while DIBAL-H was somewhat less effective but still a suitable reagent to give product in 72% yield (Table 13, entry 2). However, only trace amounts of the expected product was observed (GC-MS analysis) using LiAlH(OBu-t)₃ (Table 13, entry 3) and a hydrogenation reaction led to complete recovery of starting material (Table 13, entry 4).

Having established an effective hydride reagent, Et₃SiH, generalization of the discovered method was pursued and the results are shown in Table 14. Clearly, based on these results, Et₃SiH is an efficient reductant for the hydrodemethoxylation of 2-naphthamides and the biaryl amide (entry 3) but not benzamide derivatives.

The successful albeit lower yielding hydrodemethoxylation established using DIBAL-H (Table 13) prompted further examination of this reagent for several aromatic amides and the results are listed in Table 15. It is found that DIBAL-H is also a useful reductant with a major difference to Et₃SiH in its ability to hydrodemethoxylate not only methoxy naphthamides but also the corresponding benzamides.

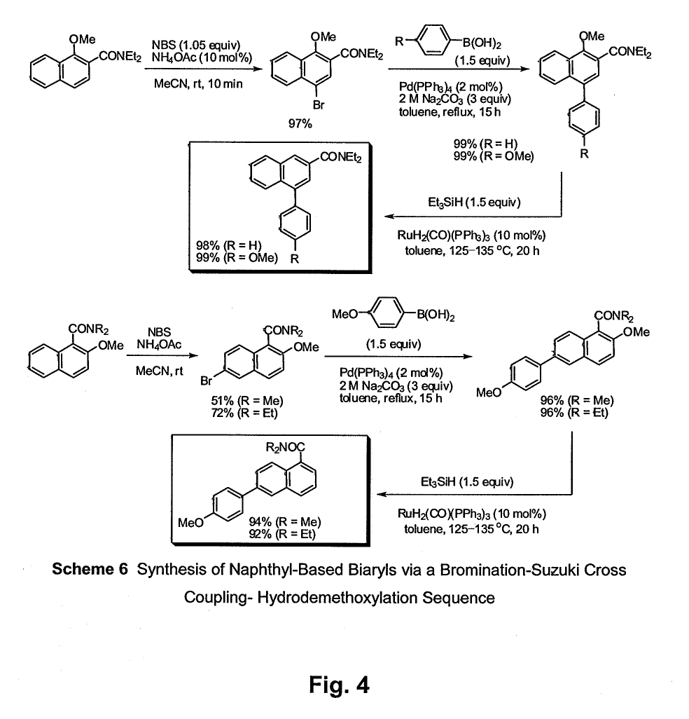

To demonstrate application of the above hydrodemethoxylation methodology, the synthesis of aryl naphthamides was carried out (see Scheme 6 in FIG. 4: Synthesis of Naphthanyl-Based Biaryls via a Bromination-Suzuki Cross Coupling-Hydrodemethoxylation Sequence). Starting from simple naphthamides, two types of naphthyl-based biaryls were synthesized in three steps in 46-95% overall yields. These syntheses demonstrate the concept, perhaps of general value, of using the strong OMe-directed electrophilic substitution reaction to derive a Suzuki coupling partner, which after it has served such a purpose, is detached to derive a substance which is again primed for further regioselective DoM chemistry.

In summary, the above studies show that the Ru-catalyzed amide-directed hydrodemethoxylation is a general method of significant potential in organic synthesis. A hydrodemethoxylation of simple aryl methyl ethers under Ni(COD)₂/PCy₃ catalytic conditions was recently reported (Alvarez-Bercedo, P.; Martin, R. J. Am. Chem. Soc. 2010, 132, 17352-17353).

### Advantages

In general, aspects of the invention provide a method that is performed under simple RuH₂(CO)(PPh₃)₃/toluene conditions with considerable advantage in high regioselectivity, yields, operational simplicity, low cost, and convenience for scale-up and handling in industrial settings. In contrast to most cross coupling reactions, neither base, additive nor organohalide are required in this process which allows minimization of waste. Starting materials are commercially available or easily prepared from inexpensive chemicals. Biaryl products can be easily transformed to useful building blocks for organic synthesis. Furthermore, the method may save steps for the preparation of some compounds which require multi-step synthesis such as the preparation of 2-aryl-1-naphthoate esters. Other advantages are described in detail as follows:
Avoidance of the conditions of the DoM (directed ortho metalation) reaction, specifically use of cryogenic temperature (usually −78° C.) and of stoichiometric to excess strong base (usually alkyllithiums). Averting the use of aryihalide coupling partners in the Suzuki cross coupling process which generates metal halide waste. The Ru-catalyzed C—O activation/coupling strategy described herein may supercede the two-step DoM-Suzuki cross coupling reaction in that it establishes a catalytic, single step replacement for the DoM-cross coupling process. Since it is carried out at non-cryogenic temperatures and under base-free conditions, it offers a convenient, economical and green alternative. This methodology, together with a method of in-situ Schwartz reduction (see Canadian patent application 2,686,915 and U.S. Patent Application Publication No. 2010/145060), promises to provide new synthetic routes for polysubstituted biaryls (see Example 17).
The catalytic and highly efficient ester-directed C—O activation/C—C bond forming reaction is demonstrated by the synthesis of 2-MeO-1-naphthoate ester. Of general value is the fact that this method establishes the most efficient and practical cross coupling route to prepare 2-substituted-1-naphthoic acid derivatives from easily available or commercial naphthalene substrates.
The new catalytic amide-directed ortho-hydrodemethoxylation reaction has potential value in links to aromatic electrophilic substitution and DoM chemistries, which establishes a new method for an aryl OMe ether group reductive cleavage. This process allows synthetic planning which involves utility of the ortho-OMe group for electrophilic bromination meta to the amide for subsequent Suzuki coupling and then its excision for potential further DoM chemistry.

### Utility of Products

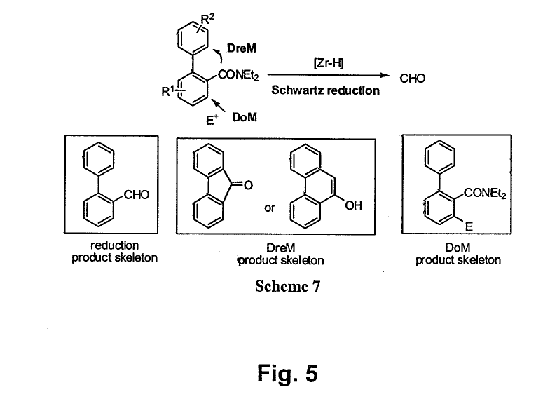

Subsequent to the Ru-catalyzed amide-directed C—O activation/arylation reaction, a rich chemistry of obtained 2-amide biaryls is presented in Scheme 7 of FIG. 5 including i) amide to aldehyde reduction using Schwartz reagent for preparation of useful building blocks (see Example 17); ii) a link to DreM (directed remote metalation) to make fused complex aromatic systems, e.g. fluorenones and phenanthrols; and iii) a large number of links to further DoM functionalization.

### WORKING EXAMPLES

The following working examples provide descriptions of syntheses that were carried out. In most cases, a representative synthetic procedure and characterization data for the representative compound are provided, followed by a table of compounds that were prepared using that procedure. For convenience, instead of sequentially numbering the tables herein, table numbers have been matched to the Example number in which they appear. Characterization data for certain compounds prepared during these studies are presented in Appendix 1.

### Example 1

### Materials

Many of the chemicals discussed below were purchased from Aldrich Chemical Company, Oakville, Ontario, Canada, which is indicated merely by the term “Aldrich”. RuH₂(CO)(PPh₃)₃ and Cp₂ZrCl₂ were purchased from Strem Chemicals, Inc. of Newburyport, Mass., USA. LiAlH(Ot-Bu)₃ was purchased from Aldrich. Silica gel 60, 230-400 mesh, was obtained from EMD Chemicals, Inc. of Darmstadt, Germany. ¹H NMR and ¹³C NMR spectra were acquired on a Varian 300 MHz and a Bruker 400 MHz spectrometers. GC-MS analyses were performed on an Agilent 6890 GC coupled with an Agilent 5973 inert MS under electron ionization conditions. High resolution MS analyses were obtained on a GCT Mass Spectrometer (available from Waters, Micromass, Manchester, England) and a QSTAR XL hybrid mass spectrometer (Applied Biosystems/MDS Sciex, Foster City, Calif., USA). IR spectra were recorded on a BOMEM FT-IR Varian 1000 FT-IR spectrometers.

### Example 2

### C—H Activation of Furan-3-Carboxamide and C—C Bond Formation

### Example 2A

### Synthesis of N,N-Diethyl-2-Phenylfuran-3-Carboxamide

This synthesis is provided as a representative example for compounds of Table 2. A mixture of N,N-diethylfuran-3-carboxamide (50 mg, 0.30 mmol), 2-phenyl-5,5-dimethyl-1,3,2-dioxaborinane (86 mg, 0.45 mmol) and RuH₂(CO)(PPh₃)₃ (11 mg, 4 mol %) in toluene (0.5 mL) was heated at 125-135° C. (oil bath temperature) in a sealed vial for 44 h. The reaction progress was monitored by GC-MS analysis. The reaction mixture was cooled to RT and concentrated in vacuo. The residue was subjected to flash SiO₂ column chromatography (eluent: EtOAc/hexanes). N,N-Diethyl-2-phenylfuran-3-carboxamide (66 mg, 90% yield) was obtained as a light yellow oil. IR (KBr) νₘₐₓ 2974, 2935, 1631, 1491, 1430, 1295, 1216, 1061, 775, 758, 692 cm⁻¹; ¹H NMR (400 MHz, CDCl₃) δ ppm: 7.66 (d, J=7.3 Hz, 2H), 7.46 (d, J=1.8 Hz, 1H), 7.37 (t, J=7.5 Hz, 2H), 7.32-7.25 (m, 1H), 6.49 (d, J=1.8 Hz, 1H), 3.58 (q, J=7.1 Hz, 2H), 3.20 (q, J=7.1 Hz, 2H), 1.26 (t, J=7.1 Hz, 3H), 0.95 (t, J=7.1 Hz, 3H); ¹³C NMR (101 MHz, CDCl₃) δ ppm: 166.21, 149.26, 141.62, 130.00, 128.59 (2C), 128.03, 125.06 (2C), 116.90, 111.59, 43.03, 39.17, 14.05, 12.53. MS EI m/z (rel. int.) 243 (M⁺, 25), 214 (10), 171 (100), 115 (10); HRMS m/z (EI, M⁺) calcd for C₁₅H₁₇NO₂, 243.1259. found 243.1261.

TABLE 2
Amide-directed C—H Activation and C—C Cross Coupling of
Aromatic and Heteroaromatic Amide

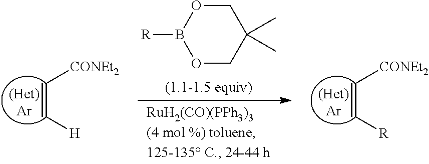

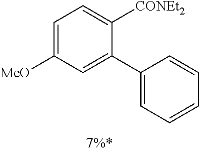

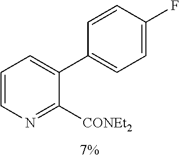

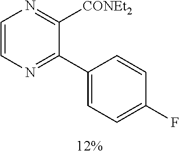

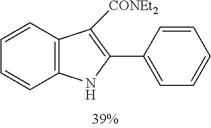

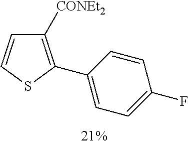

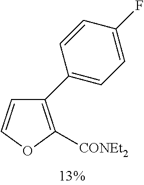

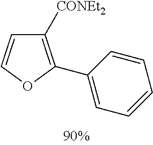

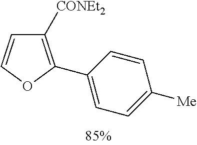

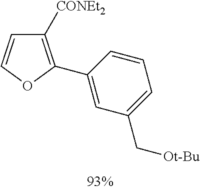

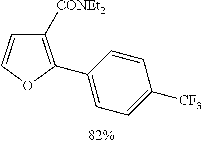

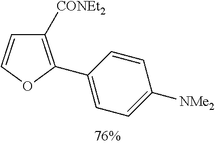

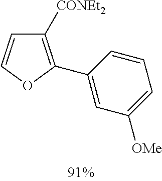

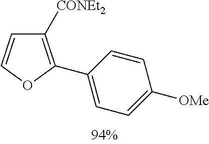

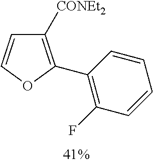

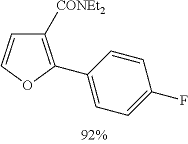

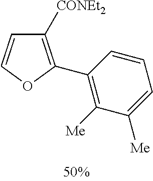

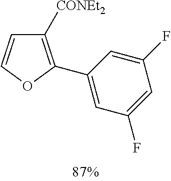

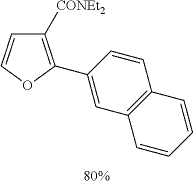

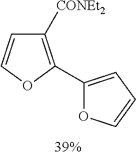

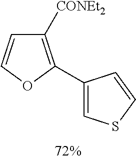

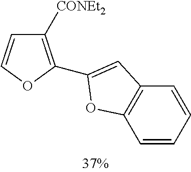

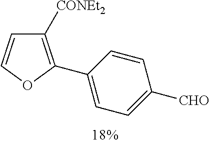

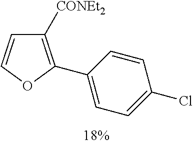

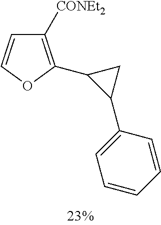

*Yields of isolated products.

### Example 3

### C—N Activation and C—C Bond Formation

### Example 3A

### Synthesis of N,N-diethyl-2-((4-trifluoromethyl)phenyl)benzamide

This synthetic procedure is provided as a representative example for compounds shown in Table 3. A mixture of N,N-diethyl-2-(dimethylamino)benzamide (66 mg, 0.30 mmol), 2((4-trifluoromethyl)phenyl)-5,5-dimethyl-1,3,2-dioxaborinane (81 mg, 0.32 mmol), RuH₂(CO)(PPh₃)₃ (11 mg, 4 mol %) in toluene (0.4 mL) was heated at 125-135° C. (oil bath temperature) in a sealed vial for 1 h. The reaction progress was monitored by GC-MS analysis. The reaction mixture was cooled to RT and concentrated in vacuo. The residue was subjected to flash SiO₂ column chromatography (eluent: EtOAc/hexanes). N,N-Diethyl-2-((4-trifluoromethyl)phenyl)benzamide (95 mg, 99% yield) was obtained as a light yellow solid. mp 81-82° C. (EtOAc/hexanes); IR (KBr) νₘₐₓ, 2977, 1628, 1430, 1326, 1290, 1165, 1125, 1109, 1069, 767 cm⁻¹; ¹H NMR (400 MHz, CDCl₃) δ ppm: 7.68-7.57 (m, 4H), 7.51-7.33 (m, 4H), 3.83-3.62 (m, 1H), 3.13-2.83 (m, 2H) 2.77-2.58 (m, 1H), 0.88 (t, J=7.1 Hz, 3H), 0.78 (t, J=7.1 Hz, 3H); ¹³C NMR (101 MHz, CDCl₃) δ ppm: 169.99, 143.39, 136.88, 136.41, 129.70 (q, ²J_{C-F}=32.7 Hz), 129.36, 129.20 (2C), 129.08, 128.32, 126.96, 125.17 (q, =3.7 Hz, 2C), 124.13 (q, ¹J_{C-F}=271.9 Hz), 42.29, 38.37, 13.42, 11.85. MS EI m/z (rel. int.) 321 (M⁺, 31), 320 (52), 249 (100), 201 (33), 152 (18); HRMS m/z (EI, M⁺) calcd for C₁₈H₁₈F₃NO, 321.1340. found 321.1334.

TABLE 3
Cross Coupling Reactions of 2-Me₂N-N,N-diethyl Benzamide
with Aryl Boroneopentylates

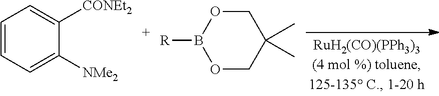

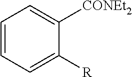

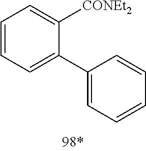

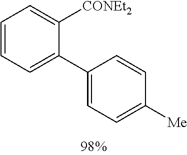

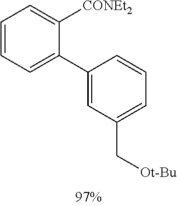

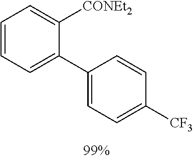

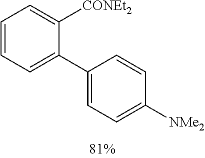

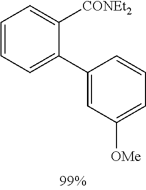

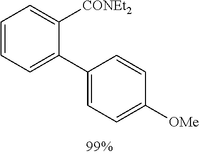

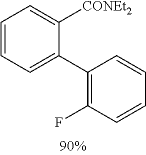

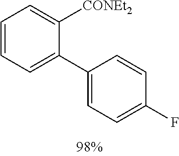

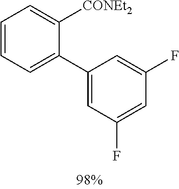

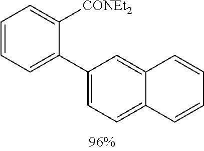

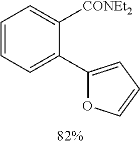

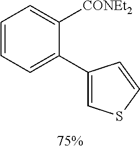

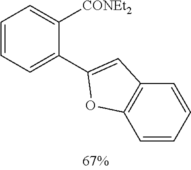

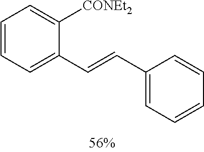

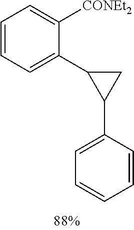

*Yields of isolated products.

### Example 4

### Screening of —OR Groups for the Cross Coupling with Phenyl Boroneopentylate

To determine whether varying the nature of the R in an alkoxy departing group, several alkoxy-substituted benzamides were studied using a particular set of reaction conditions. Results are shown in Table 4.

TABLE 4
Screening of —OR Groups for the Cross Coupling with Phenyl
Boroneopentylate

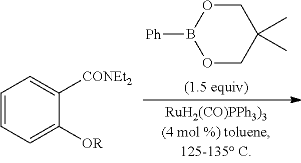

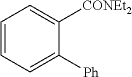

| | | | Yield |
|---|---|---|---|
| Entry | Substrate | Product | (%)^{a} |
| 1 | 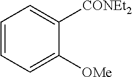 | 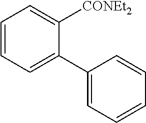 | 96 |
| 2 | 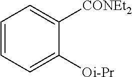 | 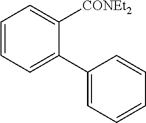 | 14^{b} |
| 3 | 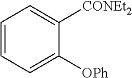 | 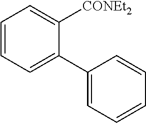 | 28^{b} |

^{a}Yields of isolated products.
^{b}Starting amide recovery: 85% (entry 2) and 72% (entry 3).

### Example 5

### Cross Coupling Reaction of the Ortho-Anisamide with Aryl Boroneopentylates

### Example 5A

### Synthesis of N,N-diethyl-2-(4-methoxyphenyl)benzamide

This synthetic procedure is provided as a representative example of compounds shown in Table 5. A mixture of N,N-diethyl-2-methoxybenzamide (62 mg, 0.30 mmol), 2-(4-methoxyphenyl)-5,5-dimethyl-1,3,2-dioxaborinane (99 mg, 0.45 mmol), RuH₂(CO)(PPh₃)₃ (11 mg, 4 mol %) in toluene (0.4 mL) was heated at 125-135° C. (oil bath temperature) in a sealed vial for 20 h. The reaction progress was monitored by GC-MS analysis. The reaction mixture was cooled to RT and concentrated in vacuo. The residue was subjected to flash SiO₂ column chromatography (eluent: EtOAc/hexanes). N,N-Diethyl-2-(4-methoxyphenyl)benzamide (83 mg, 98% yield) was obtained as light yellow solid. mp 46-47° C. (EtOAc/hexanes); IR (KBr) Vₘₐₓ 2973, 2935, 1626, 1518, 1485, 1458, 1428, 1289, 1244, 1180, 1035, 836, 764 cm⁻¹; ¹H NMR (400 MHz, CDCl₃) δ ppm: 7.46-7.29 (m, 6H), 6.90 (d, J=8.8 Hz, 2H), 3.81 (s, 3H), 3.78-3.66 (m, 1H), 3.10-2.86 (m, 2H), 2.71-2.59 (m, 1H), 0.93 (t, J=7.1 Hz, 3H), 0.73 (t, J=7.1 Hz, 3H); ¹³C NMR (101 MHz, CDCl₃) δ ppm: 170.68, 159.16, 137.90, 136.20, 132.31, 129.94 (2C), 129.23, 128.82, 127.05, 126.94, 113.66 (2C), 55.25, 42.19, 38.33, 13.36, 12.08; MS EI m/z (rel. int.) 283 (M⁺, 36), 282 (30), 211 (100), 168 (19); HRMS m/z (EI, kr) calcd for C₁₈H₂₁NO₂, 283.1572. found 283.1572.

TABLE 5
Scope of the Cross Coupling Reaction of the ortho-Anisamide
with Aryl Boroneopentylates

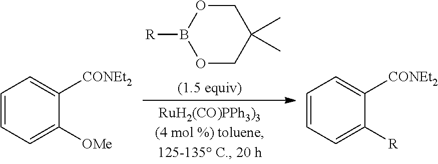

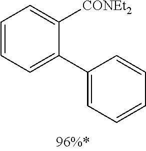

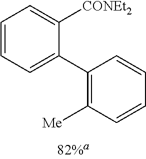

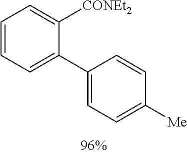

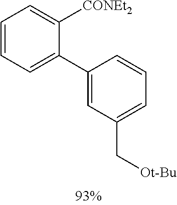

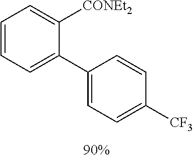

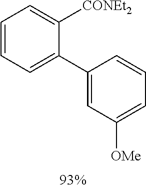

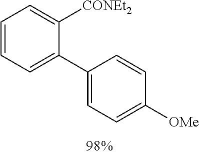

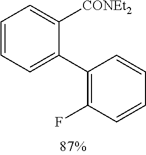

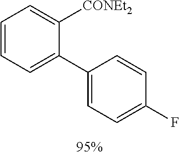

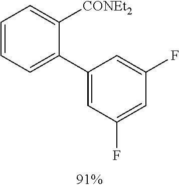

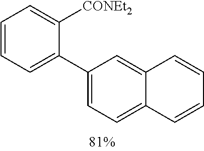

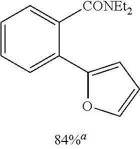

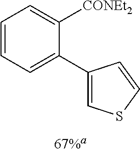

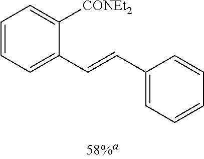

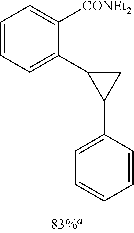

*Yields of isolated products.
^{a}The catalyst loading: 10 mol %.

### Example 6

### Cross Coupling Reaction of Substituted Ortho-Anisamides with Aryl Boroneopentylates

### Example 6A

### Synthesis of N,N-diethyl-2-phenyl-4-methoxybenzamide

This synthetic procedure is provided as a representative example for compounds shown in Table 6. A mixture of N,N-diethyl-2,4-dimethoxybenzamide (71 mg, 0.3 mmol), 2-phenyl-5,5-dimethyl-1,3,2-dioxaborinane (87 mg, 0.45 mmol), RuH₂(CO)(PPh₃)₃ (11 mg, 4 mol %) in toluene (0.8 mL) was heated at 125-135° C. (oil bath temperature) in a sealed vial for 20 h. The reaction progress was monitored by GC-MS analysis. The reaction mixture was cooled to RT and concentrated in vacuo. The residue was subjected to flash SiO₂ column chromatography (eluent: EtOAc/hexanes). N,N-Diethyl-2-phenyl-4-methoxybenzamide (76 mg, 89% yield) was obtained as a light yellow solid. mp 64-65° C. (EtOAc/hexanes); IR (KBr) νₘₐₓ 2972, 2935, 1625, 1468, 1428, 1290, 1271, 1036, 772, 702 cm⁻¹; ¹H NMR (400 MHz, CDCl₃) δ ppm: 7.47 (d, J=6.6 Hz, 2H), 7.40-7.27 (m, 4H), 6.96-6.85 (m, 2H), 3.84 (s, 3H), 3.79-3.63 (m, 1H), 3.16-2.78 (m, 2H), 2.73-2.48 (m, 1H), 0.86 (t, J=7.1 Hz, 3H), 0.72 (t, J=7.1 Hz, 3H); ¹³C NMR (101 MHz, CDCl₃) δ ppm: 170.52, 159.70, 139.97, 139.76, 129.02, 128.70 (2C), 128.44, 128.24 (2C), 127.59, 114.62, 112.97, 55.33, 42.23, 38.29, 13.35, 11.90. MS EI m/z (rel. int.) 283 (M⁺, 11), 282 (16), 211 (100); HRMS m/z (EI, M⁺) calm′ for C₁₈H₂₁NO₂, 283.1572. found 283.1574.

TABLE 6
Scope of the Cross Coupling Reaction of Substituted
ortho-Anisamides with Aryl Boroneopentylates

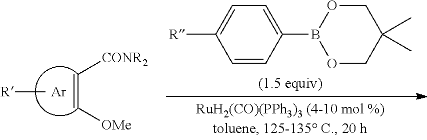

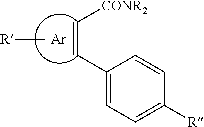

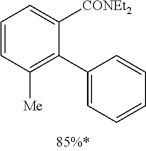

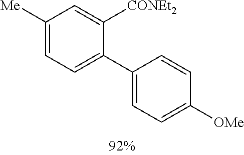

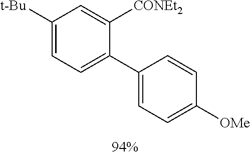

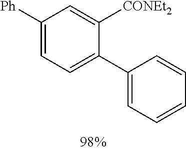

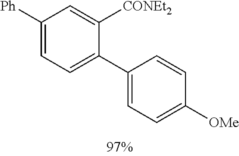

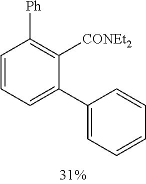

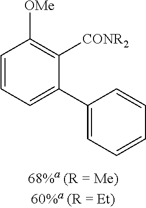

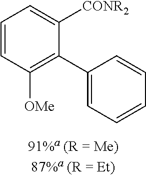

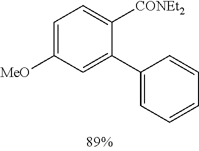

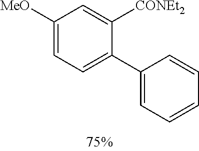

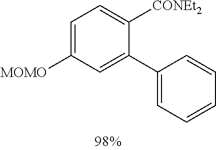

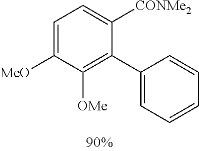

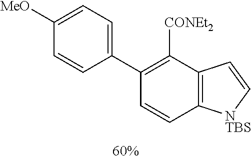

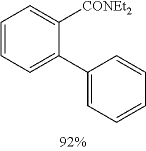

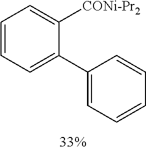

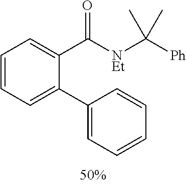

*Yields of isolated products.
^{a}Di- C—O activations were found: 22% (R = Me); 23% (R = Et).

### Example 7

### C—O Activation/Cross Coupling of Isomeric Naphthamide

As shown in Table 7, data is provided regarding C—O activation/cross coupling investigations of isomeric naphthamides.

TABLE 7
C—O Activation/Cross Coupling of Isomeric Naphthamide

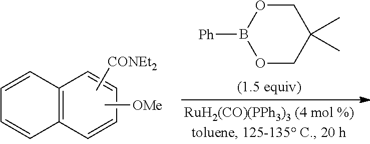

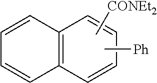

| Entry | Substrate | Product | Yield (%)^{a} |
|---|---|---|---|
| 1 | 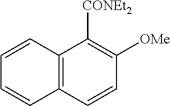 | 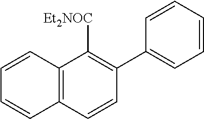 | 97 |
| 2 | 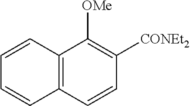 | 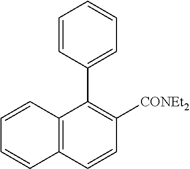 | 96 |
| 3 | 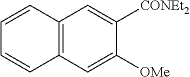 | 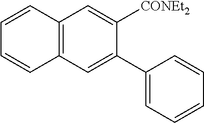 | 30 |

^{a}Yields of isolated products

### Example 8

### Cross Coupling of 1-MeO-2-Naphthamide with Aryl Boroneopentylates

Studies were conducted to determine the scope of Cross Coupling for 1-MeO-2-naphthamide with a variety of aryl boronates.

For entries 1 and 2 of Table 8, where R=Me or Et, the procedure outlined below was used and the starting material amide had the appropriate R group to provide the desired product.

### Example 8A

### Synthesis of N,N-diethyl-1-(4-methylphenyl)-2-naphthamide

This synthetic procedure is provided as a representative example for compounds shown in Table 8. A mixture of N,N-diethyl-1-methoxy-2-naphthamide (52 mg, 0.2 mmol), 2-(4-methylphenyl)-5,5-dimethyl-1,3,2-dioxaborinane (61 mg, 0.3 mmol), RuH₂(CO)(PPh₃)₃ (7 mg, 4 mol %) in toluene (0.6 mL) was heated at 125-135° C. (oil bath temperature) in a sealed vial for 20 h. The reaction progress was monitored by GC-MS analysis. The reaction mixture was cooled to RT and concentrated in vacuo. The residue was subjected to flash SiO₂ column chromatography (eluent: EtOAc/hexanes). N,N-Diethyl-1-(4-methylphenyl)-2-naphthamide (63 mg, 99% yield) was obtained as a light yellow solid. mp 181-183° C. (EtOAc/hexanes); IR (KBr) νₘₐₓ 2973, 2932, 1629, 1477, 1427, 1285, 1102, 817 cm⁻¹; ¹H NMR (400 MHz, CDCl₃) δ ppm: 7.91 (d, J=8.2 Hz, 2H), 7.74 (d, J=8.3 Hz, 1H), 7.53 (t, J=7.2 Hz, 1H), 7.49-7.37 (m, 3H), 7.33-7.18 (m, 3H), 3.95-3.71 (m, 1H), 3.25-3.06 (m, 1H), 2.98-2.82 (m, 1H), 2.81-2.65 (m, 1H), 2.44 (s, 3H), 0.91 (t, J=7.0 Hz, 3H), 0.74 (t, J=7.0 Hz, 3H); ¹³C NMR (101 MHz, CDCl₃) δ ppm: 170.34, 137.28, 135.59, 134.24, 134.11, 133.40, 132.10, 131.03, 129.55, 129.22, 127.98 (3C), 126.55, 126.43, 126.14, 123.40, 42.26, 37.78, 21.24, 13.72, 11.71. MS EI m/z (rel. int.) 317 (M⁺, 38), 316 (31), 246 (20), 245 (100), 215 (14), 202 (36); HRMS m/z (EI, M⁺) calcd for C₂₂H₂₃NO, 317.1780. found 317.1786.

TABLE 8
Cross Coupling of 1-MeO-2-naphthamide with Aryl Boroneopentylates

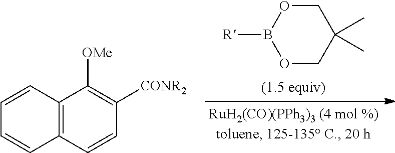

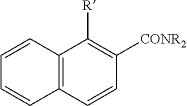

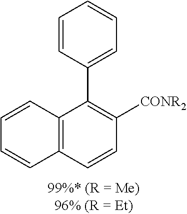

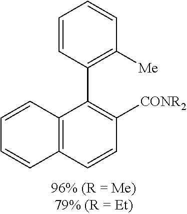

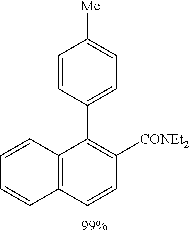

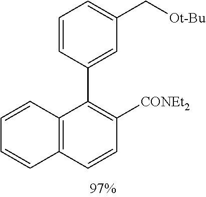

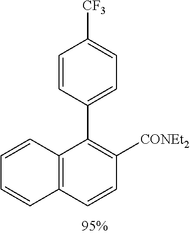

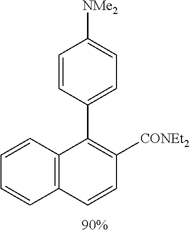

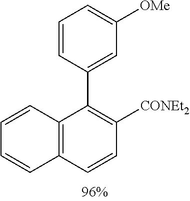

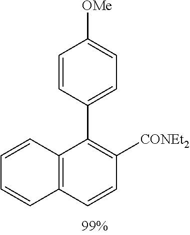

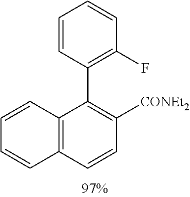

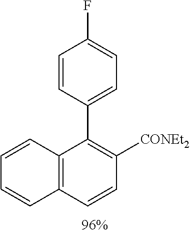

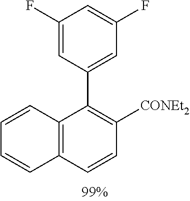

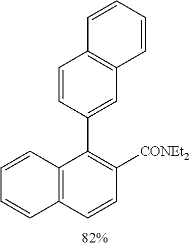

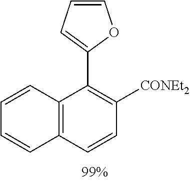

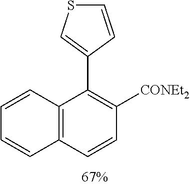

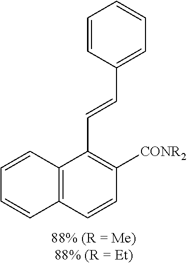

*Yields for isolated products.

### Example 9

### Cross Coupling Reaction of 2-MeO-1-Naphthamide with Aryl Boroneopentylates

### Example 9A

### Synthesis of 2-(2-fluorophenyl)-N,N-dimethyl-1-naphthamide

This synthetic procedure is provided as a representative example of compounds shown in Table 9. A mixture of N,N-dimethyl-2-methoxy-1-naphthamide (46 mg, 0.2 mmol), 2-(2-fluorophenyl)-5,5-dimethyl-1,3,2-dioxaborinane (62 mg, 0.3 mmol), RuH₂(CO)(PPh₃)₃ (7 mg, 4 mol %) in toluene (0.6 mL) was heated at 125-135° C. (oil bath temperature) in a sealed vial for 20 h. The reaction progress was monitored by GC-MS analysis. The reaction mixture was cooled to RT and concentrated in vacuo. The residue was subjected to flash SiO₂ column chromatography (eluent: EtOAc/hexanes). 2-(2-Fluorophenyl)-N,N-dimethyl-1-naphthamide (58 mg, 99% yield) was obtained as a light yellow solid. mp 105-106° C. (EtOAc/hexanes); IR (KBr) νₘₐₓ 2927, 1637, 1496, 1450, 1400, 1261, 1206, 1195, 806, 760 cm⁻¹; ¹H NMR (400 MHz, CDCl₃) δ ppm: 7.93-7.87 (m, 2H), 7.87-7.80 (m, 1H), 7.60-7.46 (m, 4H), 7.41-7.31 (m, 1H), 7.24-7.12 (m, 2H), 2.96 (s, 3H), 2.57 (s, 3H); ¹³C NMR (101 MHz, CDCl₃) δ ppm 169.50, 159.57 (d, ¹J_{C-F}=246.3 Hz), 133.74, 132.87, 131.92 (d, ⁴J_{C-F}=3.0 Hz), 130.01, 129.88, 129.70 (d, ³J_{C-F}=8.1 Hz), 128.20, 128.08, 127.97 (d, ⁴J_{C-F}=2.3 Hz), 127.36 (d, ²J_{C-F}=14.9 Hz), 127.15, 126.61, 125.45, 124.00 (d, ³J_{C-F}=3.6 Hz), 115.49 (d, ²J_{C-F}=22.1 Hz), 37.76, 34.39. MS EI m/z (rel. int.) 293 (M⁺, 28), 249 (96), 221 (38), 220 (100), 219 (20), 218 (22); HRMS m/z (EI, M⁺) calcd for C₁₉H₁₆FNO, 293.1216. found 293.1230.

TABLE 9
Cross Coupling of 2-MeO-1-naphthamide with Aryl Boroneopentylates

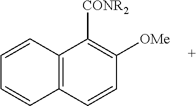

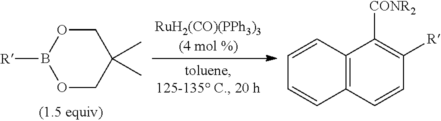

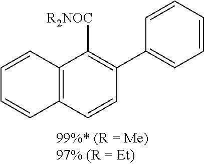

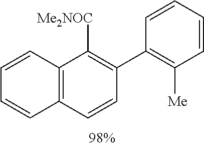

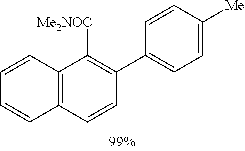

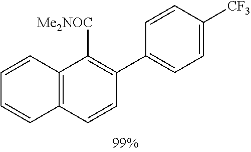

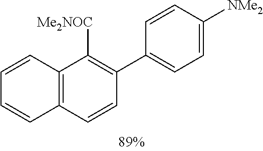

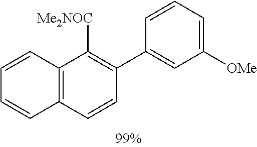

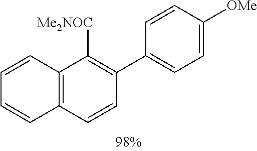

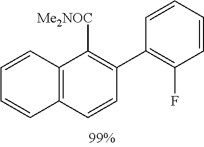

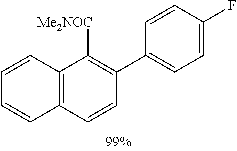

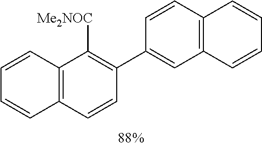

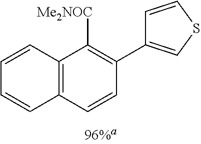

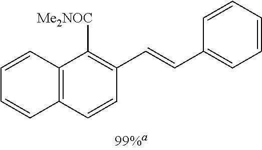

*Yields of isolated products.
^{a}The catalyst loading: 10 mol %.

### Example 10

### Selectivity in Cross Coupling of Substituted Naphthamides

Using the procedures outlined in Examples 8 and 9, investigations were conducted to probe regioselectivity preferences for cross coupling reactions of substituted naphthamides. Results are shown in Table 10.

TABLE 10
Selectivity in the Cross Coupling of Substituted Naphthamides

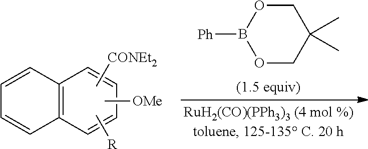

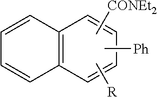

| | | | Isolated |
|---|---|---|---|
| Entry | Substrate | Product | Yield (%)^{a} |
| 1 | 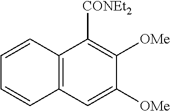 | 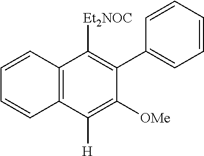 | 99 |
| 2 | 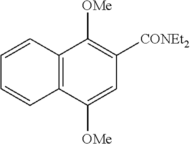 | 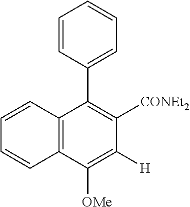 | 99 |
| 3 | 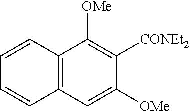 | 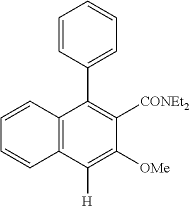 | 97 |

### Example 11

### C—O and C—N Activation/C—C Cross Coupling Reactions of Ester Directing Group Substrates

Using the procedures outlined in Example 12, investigations were conducted to probe reactivity of cross coupling reactions of aryl moieties with ester directing groups. Results are shown in Table 11.

TABLE 11
C—O Activation/C—C Cross Coupling Reactions of Ester
Directing Group Substrates

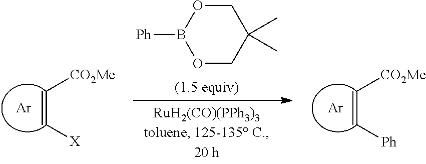

| | | Catalyst | | |
|---|---|---|---|---|
| | | loading | | Yield |
| Entry | Substrate | (mol %) | Product | (%)^{a} |
| 1 | 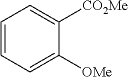 [SMILES: COC(=O)c1ccccc1OC] | 10 | 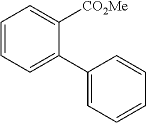 [SMILES: COC(=O)c1ccccc1-c1ccccc1] | −(4)^{b} |
| 2 | 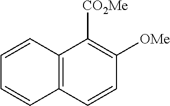 [SMILES: COC(=O)c1c(OC)ccc2ccccc12] | 4 | 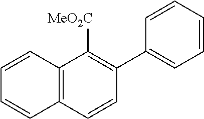 [SMILES: COC(=O)c1c(-c2ccccc2)ccc2ccccc12] | 96 |
| 3 | 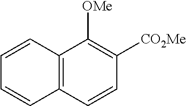 [SMILES: COC(=O)c1ccc2ccccc2c1OC] | 10 | 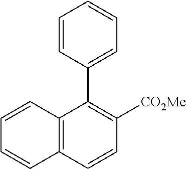 [SMILES: COC(=O)c1ccc2ccccc2c1-c1ccccc1] | 39 |
| 4 | 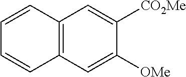 [SMILES: COC(=O)c1cc2ccccc2cc1OC] | 10 | 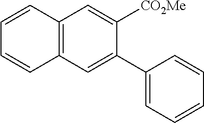 [SMILES: COC(=O)c1cc2ccccc2cc1-c1ccccc1] | n.d. |
| 5 | 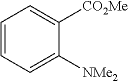 [SMILES: COC(=O)c1ccccc1N(C)C] | 10 | 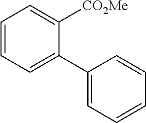 [SMILES: COC(=O)c1ccccc1-c1ccccc1] | 72 |

^{a}Yields of isolated products.
^{b}Yield determined by GC-MS analysis.

### Example 12

### C—OMe Activated Cross Coupling of Methyl 2-MeO-1-Naphthoate with Aryl Boroneopentylates

### Example 12

### Synthesis of methyl 2-(4-(trifluoromethyl)phenyl)-1-naphthoate

A mixture of methyl 2-methoxy-1-naphthoate (43 mg, 0.2 mmol), 2-(4-(trifluoromethyl)phenyl)-5,5-dimethyl-1,3,2-dioxaborinane (77 mg, 0.3 mmol), RuH₂(CO)(PPh₃)₃ (7 mg, 4 mol %) in toluene (0.4 mL) was heated at 125-135° C. (oil bath temperature) in a sealed vial for 20 h. The reaction progress was monitored by GC-MS analysis. The reaction mixture was cooled to RT and concentrated in vacuo. The residue was subjected to flash SiO₂ column chromatography (eluent: EtOAc/hexanes). Methyl 2-(4-(trifluoromethyl)phenyl)-1-naphthoate (57 mg, 86% yield) was obtained as a colorless solid. mp 74-76° C. (EtOAc/hexanes); IR (KBr) νₘₐₓ 1728, 1325, 1237, 1167, 1125, 1114, 1085, 1064, 1022, 820 cm⁻¹; ¹H NMR (400 MHz, CDCl₃) δ ppm: 8.04-7.95 (m, 2H), 7.92 (dd, J=7.5, 1.4 Hz, 1H), 7.71 (d, J=8.1 Hz, 2H), 7.65-7.54 (m, 4H), 7.49 (d, J=8.5 Hz, 1H), 3.72 (s, 3H); ¹³C NMR (101 MHz, CDCl₃) δ ppm: 169.55, 144.57, 144.56, 136.52, 132.60, 130.25, 129.89, 129.76 (q, ²J_{C-F}=32.52 Hz), 128.90, 128.18, 127.75, 126.84, 126.79, 125.35 (q, ³J_{C-F}=3.74 Hz, 2C), 125.17, 124.17 (q, J_{C-F}=272.07 Hz), 52.28. MS EI m/z (rel. int.) 330 (M⁺, 62), 299 (100), 251 (29), 202 (65), 69 (65); HRMS m/z (EI, M⁺) calcd for C₁₉H₁₃F₃O₂, 330.0868. found 330.0848.

TABLE 12
C—OMe Activated Cross Coupling of Methyl 2-MeO-1
naphthoate with Aryl Boroneopentylates

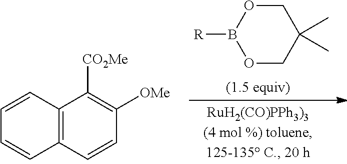

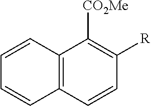

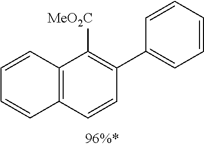 [SMILES: COC(=O)c1c(-c2ccccc2)ccc2ccccc12]

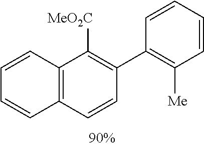 [SMILES: COC(=O)c1c(-c2ccccc2C)ccc2ccccc12]

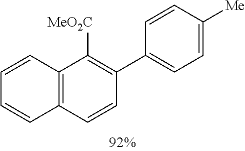 [SMILES: COC(=O)c1c(-c2ccc(C)cc2)ccc2ccccc12]

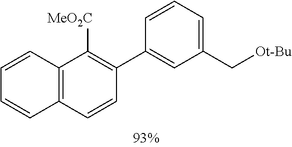 [SMILES: COC(=O)c1c(-c2cccc(COC(C)(C)C)c2)ccc2ccccc12]

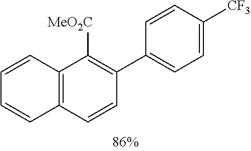 [SMILES: COC(=O)c1c(-c2ccc(C(F)(F)F)cc2)ccc2ccccc12]

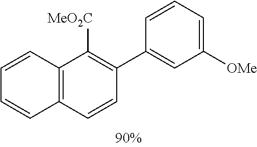 [SMILES: COC(=O)c1c(-c2cccc(OC)c2)ccc2ccccc12]

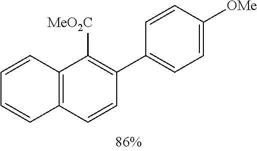 [SMILES: COC(=O)c1c(-c2ccc(OC)cc2)ccc2ccccc12]

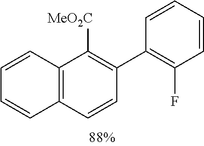 [SMILES: COC(=O)c1c(-c2ccccc2F)ccc2ccccc12]

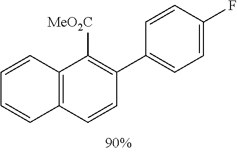 [SMILES: COC(=O)c1c(-c2ccc(F)cc2)ccc2ccccc12]

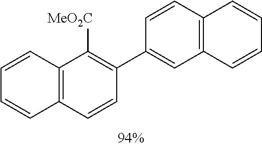 [SMILES: COC(=O)c1c(-c2ccc3ccccc3c2)ccc2ccccc12]

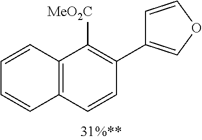 [SMILES: COC(=O)c1c(-c2ccoc2)ccc2ccccc12]

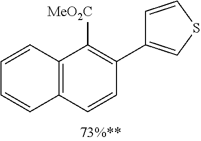 [SMILES: COC(=O)c1c(-c2ccsc2)ccc2ccccc12]

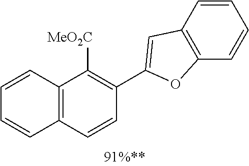 [SMILES: COC(=O)c1c(-c2cc3ccccc3o2)ccc2ccccc12]

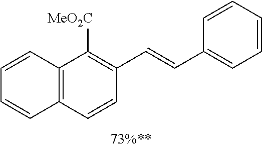 [SMILES: COC(=O)c1c(/C=C/c2ccccc2)ccc2ccccc12]

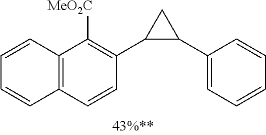 [SMILES: COC(=O)c1c(C2CC2c2ccccc2)ccc2ccccc12]

*Yields of isolated products.
**10 mol % catalyst loading.

### Example 13

### Screening of Reductants

Studies were conducted to probe efficacy of several reductants using a model reaction of cross coupling of 1-MeO-2-naphthamide. Results are shown in Table 13. Notably, Si—H and Al—H reductants were effective. In contrast, LiAlH(OBu-t)₃ and hydrogen were not effective in this particular reaction.

TABLE 13
Initial Test for Reductants

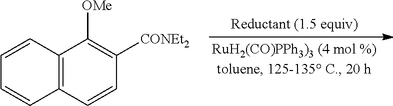

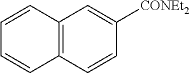

| Entry | Reductant | Isolated Yield (%) |
|---|---|---|
| 1 | Et₃SiH | 98 |
| 2 | DIBAL-H | 72 |
| 3 | LiAlH(OBu-t)₃ | −(9)^{a} |
| 4 | H₂ | −^{b} |

^{a}Yield determined by GC-MS analysis
^{b}60 psi. Recovery of staring material (98%).

### Example 14

### Ru-Catalyzed Hydrodemethoxylation of Benzamides and Naphthamides Using Et₃SiH

### Example 14A

### Synthesis of N,N-diethyl-4-methoxy-2-naphthamide

This synthetic procedure is provided as a representative example of compounds shown in Table 14. A mixture of N,N-diethyl-1,4-dimethoxy-2-naphthamide (58 mg, 0.2 mmol), Et₃SiH (36 mg, 0.3 mmol), RuH₂(CO)(PPh₃)₃ (7 mg, 4 mol %) in toluene (0.6 mL) was heated at 125-135° C. (oil bath temperature) in a sealed vial for 20 h. The reaction progress was monitored by GC-MS analysis. The reaction mixture was cooled to RT and concentrated in vacuo. The residue was subjected to flash SiO₂ column chromatography (eluent: EtOAc/hexanes). N,N-Diethyl-4-methoxy-2-naphthamide (49 mg, 93% yield) was obtained as a light yellow oil. IR (KBr) νₘₐₓ, 2971, 2935, 1627, 1597, 1577, 1478, 1459, 1422, 1397, 1372, 1293, 1266, 1235, 1111, 1095, 818, 779 cm⁻¹; ¹H NMR (400 MHz, CDCl₃) δ ppm: 8.25 (dd, J=6.9, 2.3 Hz, 1H), 7.79 (dd, J=6.8, 2.1 Hz, 1H), 7.59-7.46 (m, 2H), 7.41 (s, 1H), 6.81 (s, 1H), 4.02 (s, 3H), 3.70-3.15 (m, 4H), 1.41-1.08 (m, 6H); ¹³C NMR (101 MHz, CDCl₃) δ ppm: 171.34, 155.65, 134.57, 133.64, 127.80, 127.00, 125.96, 125.60, 121.91, 117.66, 102.16, 55.60, 43.03, 39.00, 14.10, 12.82. MS EI m/z (rel. int.) 257 (Kr, 85), 242 (40), 186 (32), 185 (100), 158 (32), 157 (47), 114 (22); HRMS m/z (EI, Kr) calcd for C₁₆H₁₉NO₂, 257.1416. found 257.1424.

TABLE 14
Ru-catalyzed Hydrodemethoxylation Benzamides and Naphthamides Using Et₂SiH

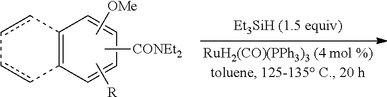

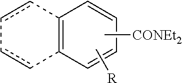

| | | | Yield |
|---|---|---|---|
| Entry | Substrate | Product | (%)^{a} |
| 1 | 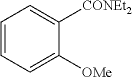 | 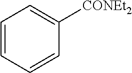 | −(4)^{b} |
| 2 | 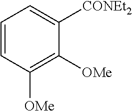 | 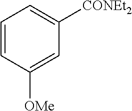 | −(12)^{b} |
| 3 | 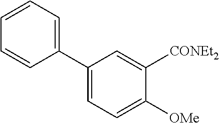 | 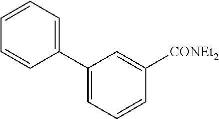 | 88^{c} |
| 4 | 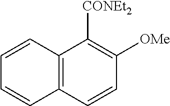 | 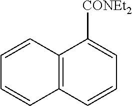 | 87 |
| 5 | 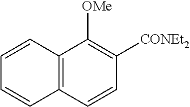 | 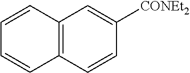 | 98 |
| 6 | 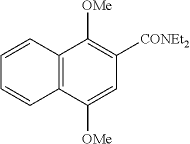 | 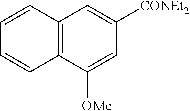 | 93 |

^{a}Yields of isolated products.
^{b}Yield determined by GC-MS analysis
^{c}The catalyst loading: 10 mol %

### Example 15

### Ru-Catalyzed Hydrodemethoxylation Using DIBAL-H

### Example 15A

### Synthesis of N,N-diethyl-2-naphthamide

This synthetic procedure is provided as a representative example of compounds shown in Table 15. A mixture of N,N-diethyl-1-methoxy-2-naphthamide (52 mg, 0.20 mmol), DIBAL-H (0.22 mL, 0.22 mmol, 1 M in THF), RuH₂(CO)(PPh₃)₃ (7 mg, 4 mol %) in toluene (0.6 mL) was heated at 125-135° C. (oil bath temperature) in a sealed vial for 20 h. The reaction progress was monitored by GC-MS analysis. The reaction mixture was cooled to RT and concentrated in vacuo. The residue was subjected to flash SiO₂ column chromatography (eluent: EtOAc/hexanes). N,N-Diethyl-2-naphthamide (38 mg, 83% yield) was obtained as a light yellow oil. ¹H NMR (400 MHz, CDCl₃) δ ppm: 7.93-7.79 (m, 4H), 7.57-7.49 (m, 2H), 7.47 (dd, J=8.4, 1.3 Hz, 1H), 3.74-3.47 (m, 2H), 3.43-3.16 (m, 2H), 1.42-1.21 (m, 3H), 1.20-0.99 (m, 3H); ¹³C NMR (101 MHz, CDCl₃) δ ppm: 171.21, 134.57, 133.31, 132.72, 128.23, 128.18, 127.71, 126.68, 126.51, 125.67, 123.87, 43.32, 39.23, 14.20, 12.93. The physical and spectral data were consistent with those previously reported (Salvio, R.; Moisan, L.; Ajami, D.; Rebek, J. Eur. J. Org. Chem. 2007, 2722-2728).

TABLE 15
Ru-catalyzed Hydrodemethoxylation Using DIBAL-H

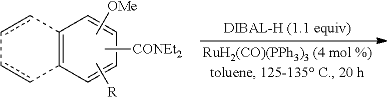

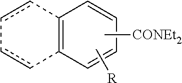

| Entry | Substrate | Product | Yield (%)^{a} |
|---|---|---|---|
| 1 | 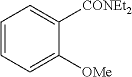 | 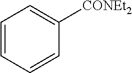 | 51 |
| 2 | 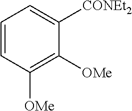 | 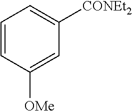 | 68 |
| 3 | 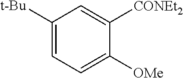 | 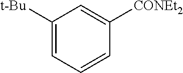 | 55^{b} |
| 4 | 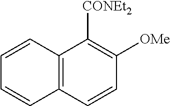 | 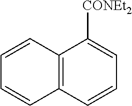 | 70^{c} |
| 5 | 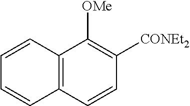 | 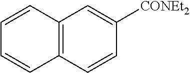 | 83 72^{c} |

^{a}Yields of isolated products.
^{b}The catalyst loading: 10 mol %
^{c}1.5 Equiv. of reductant is used

### Example 16

### Procedures for Bromination and Suzuki Cross Coupling Steps in Schemes 3, 4, 5 and 6 of FIGS. 1, 3 and 4

### Example 16A

### Synthesis of 5-bromo-2-(dimethylamino)-N,N-diethylbenzamide

To a mixture of N,N-diethyl-2-(dimethylamino)benzamide (221 mg, 1.00 mmol) and NH₄OAc (8 mg, 0.10 mmol) in MeCN (5 mL) at RT was added NBS (189 mg, 1.05 mmol) quickly. The reaction was stirred at RT for 2 min and monitored by TLC analysis until the completion. After removal of the solvent, water and EtOAc were added to the residue, the layers were separated and the water layer was extracted with EtOAc. The combined organic extract was washed with brine, dried (MgSO₄) and concentrated in vacuo. The residue was subjected to flash SiO₂ column chromatography (eluent: EtOAc/hexanes). 5-Bromo-2-(dimethylamino)-N,N-diethylbenzamide (268 mg, 90% yield) was obtained as a colorless oil. ¹H NMR (400 MHz, CDCl₃) δ ppm 7.33 (dd, J=8.7, 2.3 Hz, 1H), 7.26 (d, J=2.3 Hz, 1H), 6.76 (d, J=8.7 Hz, 1H), 3.83-3.62 (m, 1H), 3.43-3.26 (m, 1H), 3.25-2.98 (m, 2H), 2.77 (s, 6H), 1.22 (t, J=7.1 Hz, 3H), 1.03 (t, J=7.1 Hz, 3H); ¹³C NMR (101 MHz, CDCl₃) δ ppm 169.67, 148.27, 132.18, 131.08, 118.62, 112.74, 43.31 (2C), 42.75, 38.89, 13.69, 12.50 (1C not observed). The physical and spectral data were consistent with those previously reported (Stanetty, P.; Krumpak, B.; Rodler, I. K. J. Chem. Res., Synop. 1995, 342-343).

### Example 16B

### Synthesis of 4-bromo-N,N-diethyl-1-methoxy-2-naphthamide

To a mixture of N,N-diethyl-1-methoxy-2-naphthamide (515 mg, 2.0 mmol) and NH₄OAc (15 mg, 0.2 mmol) in MeCN (10 mL) at RT was added NBS (378 mg, 2.1 mmol) quickly. The reaction was stirred at RT for 10 min and monitored by TLC analysis until the completion. After removal of the solvent, water and EtOAc were added to the residue, the layers were separated and the water layer was extracted with EtOAc. The combined organic extract was washed with brine, dried (MgSO₄) and concentrated in vacuo. The residue was subjected to flash SiO₂ column chromatography (eluent: EtOAc/hexanes). 4-Bromo-N,N-diethyl-1-methoxy-2-naphthamide (650 mg, 97% yield) was obtained as a yellow oil. IR (KBr) νₘₐₓ 2973, 2935, 1634, 1592, 1476, 1454, 1429, 1361, 1324, 1278, 1255, 1220, 1132, 1083, 763 cm⁻¹; ¹H NMR (400 MHz, CDCl₃) δ ppm 8.20 (d, J=9.1 Hz, 1H), 8.18 (d, J=9.1 Hz, 1H), 7.68-7.51 (m, 3H), 4.00 (s, 3H), 3.86-3.69 (m, 1H), 3.53-3.35 (m, 1H), 3.32-3.08 (m, 2H), 1.30 (t, J=7.1 Hz, 3H), 1.05 (t, J=7.1 Hz, 3H); ¹³C NMR (101 MHz, CDCl₃) δ ppm 167.49, 151.45, 132.97, 128.95, 128.19, 128.15, 127.41, 127.11, 126.25, 122.87, 117.54, 62.77, 43.15, 39.18, 14.02, 12.74. MS EI m/z (rel. int.) 337 ([M+2]⁺, 14), 335 (M⁺, 17), 265 (89), 263 (87), 250 (24), 248 (25), 194 (26), 192 (30), 156 (23), 155 (24), 128 (30), 127 (23), 126 (65), 113 (62), 72 (31), 58 (34), 57 (100), 56 (100); HRMS m/z (ESI, [M+1]⁺) calcd for C₁₆H₁₉Br NO₂, 336.0599. found 336.0590.

### Example 16C

### Synthesis of 2-(dimethylamino)-5-phenyl-N,N-diethylbenzamide

A mixture of 5-bromo-2-(dimethylamino)-N,N-diethylbenzamide (180 mg, 0.6 mmol), phenylboronic acid (110 mg, 0.9 mmol), a degassed 2 M aqueous solution of Na₂CO₃ (0.9 mL, 1.8 mmol) and Pd(PPh₃)₄ (14 mg, 2 mol %) and toluene (1 mL) was heated at 120-130° C. (oil bath temperature) in a sealed vial for 15 h. The reaction progress was monitored by GC-MS analysis. The reaction mixture was cooled to RT and extracted with EtOAc. Then, the combined organic extract was washed with brine, dried (MgSO₄) and concentrated in vacuo. The residue was subjected to flash SiO₂ column chromatography (eluent: EtOAc/hexanes). 2-(Dimethylamino)-5-phenyl-N,N-diethylbenzamide (157 mg, 89% yield) was obtained as a light yellow oil. IR (KBr) νₘₐₓ 2973, 2936, 1625, 1515, 1486, 1458, 1432, 1378, 1320, 1263, 1137, 1081, 763, 699 cm⁻¹; ¹H NMR (400 MHz, CDCl₃) δ ppm 7.56 (d, J=7.3 Hz, 2H), 7.51 (dd, J=8.4, 2.0 Hz, 1H), 7.43 (d, J=2.0 Hz, 1H), 7.40 (t, J=7.6 Hz, 2H), 7.28 (t, J=7.4 Hz, 1H), 6.96 (d, J=8.4 Hz, 1H), 3.90-3.71 (m, 1H), 3.42-3.31 (m, 1H), 3.30-3.19 (m, 1H), 3.18-3.06 (m, 1H), 2.85 (s, 6H), 1.26 (t, J=7.1 Hz, 3H), 1.03 (t, J=7.1 Hz, 3H); ¹³C NMR (101 MHz, CDCl₃) 6 ppm 171.25, 148.50, 140.22, 133.06, 129.52, 128.65 (2C), 127.89, 127.05, 126.64, 126.46 (2C), 117.15, 43.38 (2C), 42.75, 38.81, 13.75, 12.55. MS EI m/z (rel. int.) 296 (M⁺, 38), 224 (100), 223 (50), 196 (25), 181 (47), 180 (36), 167 (38), 153 (42), 152 (75), 72 (41), 58 (48), 57 (38), 56 (66); HRMS m/z (ESI, [M+1]⁺) calcd for C₁₉H₂₅N₂O, 297.1966. found 297.1979.

### Example 16D

### Synthesis of N,N-diethyl-1-methoxy-4-(4-methoxyphenyl)-2-naphthamide

A mixture of 4-bromo-N,N-diethyl-1-methoxy-2-naphthamide (135 mg, 0.4 mmol), 4-methoxyphenylboronic acid (91 mg, 0.6 mmol), a degassed 2 M aqueous solution of Na₂CO₃ (0.6 mL, 1.2 mmol) and Pd(PPh₃)₄ (9 mg, 2 mol %) and toluene (0.6 mL) was heated at 120-130° C. (oil bath temperature) in a sealed vial for 15 h. The reaction progress was monitored by GC-MS analysis. The reaction mixture was cooled to RT and extracted with EtOAc. Then, the combined organic extract was washed with brine, dried (MgSO₄) and concentrated in vacuo. The residue was subjected to flash SiO₂ column chromatography (eluent: EtOAc/hexanes). N,N-Diethyl-1-methoxy-4-(4-methoxyphenyl)-2-naphthamide (143 mg, 99% yield) was obtained as a light yellow solid. mp 129-130° C. (EtOAc/hexanes); IR (KBr) νₘₐₓ 2972, 2935, 1632, 1610, 1515, 1476, 1458, 1430, 1370, 1272, 1248, 1222, 1177, 1062, 1033, 839, 773 cm⁻¹; ¹H NMR (400 MHz, CDCl₃) δ ppm 8.23 (d, J=8.3 Hz, 1H), 7.91 (d, J=8.3 Hz, 1H), 7.55 (t, J=7.5 Hz, 1H), 7.47 (t, J=7.6 Hz, 1H), 7.40 (d, J=8.6 Hz, 2H), 7.25 (s, 1H), 7.02 (d, J=8.6 Hz, 2H), 4.05 (s, 3H), 3.88 (s, 3H), 3.85-3.73 (m, 1H), 3.57-3.39 (m, 1H), 3.37-3.11 (m, 2H), 1.31 (t, J=7.1 Hz, 3H), 1.07 (t, J=7.1 Hz, 3H); ¹³C NMR (101 MHz, CDCl₃) δ ppm 168.95, 158.97, 150.88, 136.34, 133.18, 132.22, 131.10 (2C), 128.02, 126.73, 126.41, 126.13, 125.42, 125.35, 122.61, 113.71 (2C), 62.74, 55.32, 43.17, 39.09, 14.11, 12.83. MS EI m/z (rel. int.) 363 (M⁺, 36), 291 (100), 205 (24), 189 (47), 177 (27), 176 (33), 56 (33); HRMS m/z (EI, M⁺) calcd for C₂₃H₂₅NO₃, 363.1834. found 363.1834.

### Example 17

### Reduction of Amides to Aldehydes by an In Situ-Generated Schwartz Reagent

Following use of the amide directing group to modify an aryl ring, it is possible to convert the amide to an aldehyde. Advantages of such a conversion include the versatility of aldehydes. Aldehydes can be converted to a variety of other functional groups. Details of this process are described in U.S. Patent Application Publication No. 2010-0145060 (U.S. Pat. No. 8,168,833).

Briefly, methods are provided for performing selective reductions of substrates without the necessity of pre-preparing Schwartz Reagent. This one-step method mixes three compounds. However, two of the mixed compounds do not react with the third, instead they selectively react with each other. Their reaction leads to formation of an intermediate reaction product that is only briefly present in the mixture. The reason for the briefness of its presence is that it is selectively reactive toward the third compound in the mixture. Upon reaction of the intermediate reaction product with this third compound, a desired end product is formed. Thus three compounds, A, B and D, are all provided in a mixture. A and B react to form an intermediate product, which then reacts with substrate D. A desired product is formed from the reaction of the intermediate product and D. The product is a reduced form of D and is known herein as E. To assist with completeness and speed of reaction, a solvent is also present to solubilize the mixture. A is Schwartz Reagent Precursor, Cp₂ZrCl₂, which is significantly less expensive to purchase than Schwartz Reagent. B is a reducing agent that is selective for A. In certain embodiments of the invention, B is LiAlH(OBu-O₃, LiBH(s-Bu)₃, or a combination thereof. These reducing agents are inert to many functional groups and are selective for others. A-selective reductants did not undergo substantially any side reactions with D when D was tertiary amide, tertiary benzamide, aryl O-carbamate, or heteroaryl N-carabamate. Nor did the reductants undergo reactions with any intermediates formed during these reactions. As noted above, D is substrate. Examples of D include tertiary amides, tertiary benzamides, aryl O-carbamates, N-carbamates, and aryl N-carbamates including heteroaryl N-carbamates. As noted above, E is the reaction product of the reduction of substrate, D. Examples of E include aldehydes, benzaldehydes, aromatic alcohols (commonly referred to as phenols), and N-heteroaromatic compounds.

Accordingly, substituted benzamides that have been provided by activating and C—C cross coupling methods described herein can have their amide moiety converted to aldehydes, benzaldehydes, aromatic alcohols (commonly referred to as phenols), and N-heteroaromatic compounds.

### Example 17A

### Synthesis of 1-(3-methoxyphenyl)-2-naphthaldehyde

This synthetic procedure is provided an a representative example of a conversion that may be effective for substantially all of the benzamides described herein. To a solution of N,N-diethyl-1-(3-methoxyphenyl)-2-naphthamide (17 mg, 0.05 mmol) and Cp₂ZrCl₂ (21 mg, 0.07 mmol) in THF (0.5 mL) at RT was rapidly added a 1 M THF solution of LiAlH(Ot-Bu)₃ (0.07 mL, 0.07 mmol). The resulting solution was stirred at RT for 2 min and the reaction was monitored by TLC analysis. The reaction mixture was immediately quenched by H₂O. A solution of 0.5 N HCl was added to adjust the pH<7 and the whole was extracted with EtOAc or ether. The combined organic extract was washed with brine, dried (MgSO₄) and concentrated in vacuo. The residue was subjected to flash SiO₂ column chromatography (eluent: EtOAc/hexanes). 1-(3-Methoxyphenyl)-2-naphthaldehyde (12 mg, 90% yield) was obtained (see Table 17) as a light yellow oil. IR (KBr)νₘₐₓ 2850, 1692, 1678, 1597, 1577, 1487, 1462, 1429, 1286, 1256, 1224, 1046, 821, 781, 764, 749 cm⁻¹; ¹H NMR (400 MHz, CDCl₃) δ ppm 9.92 (s, 1H), 8.06 (d, J=8.6 Hz, 1H), 7.94 (d, J=8.6 Hz, 1H), 7.93 (d, J=7.8 Hz, 1H), 7.70 (d, J=8.5 Hz, 1H), 7.62 (t, J=7.5 Hz, 1H), 7.51-7.40 (m, 2H), 7.07 (dd, J=8.0, 2.1 Hz, 1H), 7.00 (d, J=7.4 Hz, 1H), 6.96 (s, 1H), 3.85 (s, 3H); ¹³C NMR (101 MHz, CDCl₃) δ ppm 192.69, 159.36, 146.33, 136.55, 136.05, 132.34, 131.09, 129.30, 128.75, 128.33, 128.17, 127.70, 126.86, 123.54, 122.03, 116.59, 113.94, 55.33. MS EI m/z (rel. int.) 262 (M⁺, 100), 261 (36), 233 (28), 231 (44), 203 (42), 202 (31), 201 (28), 189 (45), 149 (43); HRMS m/z (EI, M⁺) calcd for C₁₈H₁₄O₂, 262.0994. found 262.0994.

### Example 17B

### Synthesis of 1-(naphthalen-2-yl)-2-naphthaldehyde

This synthetic procedure is provided an a representative example of a conversion that may be effective for substantially all of the benzamides described herein. To a solution of N,N-diethyl-1-(naphthalen-2-yl)-2-naphthamide (18 mg, 0.05 mmol) and Cp₂ZrCl₂ (21 mg, 0.07 mmol) in THF (0.5 mL) at RT was rapidly added a 1 M THF solution of LiAlH(Ot-Bu)₃ (0.07 mL, 0.07 mmol). The resulting solution was stirred at RT for 2 min and the reaction was monitored by TLC analysis. The reaction mixture was immediately quenched by H₂O. A solution of 0.5 N HCl was added to adjust the pH<7 and the whole was extracted with EtOAc or ether. The combined organic extract was washed with brine, dried (MgSO₄) and concentrated in vacuo. The residue was subjected to flash SiO₂ column chromatography (eluent: EtOAc/hexanes). 1-(Naphthalen-2-yl)-2-naphthaldehyde (13 mg, 89% yield) was obtained (see Table 17) as a light yellow viscous oil. IR (KBr) νₘₐₓ 3058, 2849, 1689, 1678, 1228, 821, 765, 747 cm⁻¹; ¹H NMR (400 MHz, CDCl₃) δ ppm 9.92 (s, 1H), 8.11 (d, J=8.6 Hz, 1H), 8.05-7.93 (m, 4H), 7.92-7.82 (m, 2H), 7.68 (d, J=8.5 Hz, 1H), 7.65-7.57 (m, 3H), 7.54 (dd, J=8.3, 1.2 Hz, 1H), 7.44 (t, J=7.5 Hz, 1H); ¹³C NMR (101 MHz, CDCl₃) δ ppm 192.63, 146.41, 136.09, 132.92, 132.81, 132.65, 132.57, 131.48, 130.42, 128.77, 128.61, 128.45, 128.26, 128.06, 127.89 (2C), 127.79, 126.94, 126.91, 126.76, 122.18. MS EI m/z (rel. int.) 282 (M⁺, 100), 281 (54), 253 (42), 252 (56), 149 (21), 126 (37); HRMS m/z (EI, M⁺) calcd for C₂₁H₁₄O, 282.1045. found 282.1049.

TABLE 17
Reduction of Amides to Aldehydes via the in situ Schwartz Method

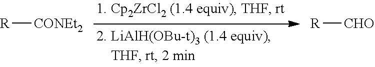

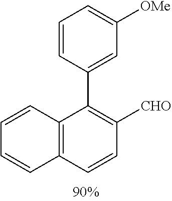 [SMILES: COc1cccc(-c2c(C=O)ccc3ccccc23)c1]

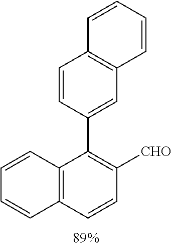 [SMILES: O=Cc1ccc2ccccc2c1-c1ccc2ccccc2c1]

*Yields of isolated and purified products.

It will be understood by those skilled in the art that this description is made with reference to certain preferred embodiments and that it is possible to make other embodiments employing the principles of the invention which fall within its spirit and scope as defined by the claims.

### APPENDIX 1

### Characterization Data for Indicated Compounds

### N,N-Diethyl-3-(4-fluorophenyl)picolinamide

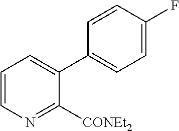

Light yellow oil. IR (KBr) 2977, 1636, 1513, 1223, 1103, 798 cm⁻; ¹H NMR (400 MHz, CDCl₃) δ ppm 8.62 (dd, J=4.7, 1.5 Hz, 1H), 7.72 (dd, J=7.8, 1.5 Hz, 1H), 7.52-7.44 (m, 2H), 7.38 (dd, J=7.8, 4.8 Hz, 1H), 7.15-7.01 (m, 2H), 3.42 (q, J=7.1 Hz, 2H), 2.89 (q, J=7.1 Hz, 2H), 1.01 (t, J=7.1 Hz, 3H), 0.87 (t, J=7.1 Hz, 3H); ¹³C NMR (101 MHz, CDCl₃) δ ppm 168.07, 162.84 (d, ¹J_{C-F}=248.3 Hz), 153.73, 148.34, 137.24, 133.28, 133.21 (d, =3.4 Hz), 130.63 (d, ³J_{C-F}=8.1 Hz, 2C), 123.61, 115.57 (d, ²J_{C-F}=21.5 Hz, 2C), 42.46, 38.72, 13.50, 12.21. MS EI m/z (rel. int.) 272 (M⁺, 7), 173 (13), 172 (23), 72 (100); HRMS m/z (EI, M⁺) calcd for C₁₆H₁₇FN₂O, 272.1325. found 272.1319.

### N,N-Diethyl-3-(4-fluorophenyl)pyrazine-2-carboxamide

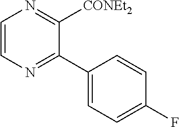

Yellow oil. IR (KBr) νₘₐₓ 2978, 2936, 1638, 1513, 1382, 1227, 1161, 1111, 848 cm⁻; ¹H NMR (400 MHz, CDCl₃) δ ppm 8.67 (d, J=2.4 Hz, 1H), 8.54 (d, J=2.4 Hz, 1H), 7.88-7.77 (m, 2H), 7.14 (t, J=8.6 Hz, 2H), 3.50 (q, J=7.1 Hz, 2H), 2.92 (q, J=7.1 Hz, 2H), 1.14 (t, J=7.1 Hz, 3H), 0.88 (t, J=7.1 Hz, 3H); ¹³C NMR (101 MHz, CDCl₃) δ ppm 167.08, 163.82 (d, ¹J_{C-F}=250.4 Hz), 149.82, 148.89, 144.09, 142.06, 132.54 (d, ⁴J_{C-F}=3.3 Hz), 130.82 (d, ³J_{C-F}=8.5 Hz, 2C), 115.71 (d, ²J_{C-F}=21.7 Hz, 2C), 42.66, 39.15, 13.42, 12.14. MS EI m/z (rel. int.) 273 (M⁺, 6), 173 (18), 72 (100); HRMS m/z (EI, M⁺) calcd for C₁₅H₁₆FN₃O, 273.1277. found 273.1277.

### N,N-Diethyl-2-phenyl-1H-indole-3-carboxamide

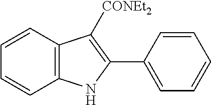

Light yellow solid. mp 224-226° C. (EtOAc/hexanes); IR (KBr) νₘₐₓ 3143, 2976, 2930, 1594, 1574, 1543, 1495, 1457, 1420, 1320, 1274, 1235, 1124, 1048, 743, 696 cm⁻¹; ¹H NMR (400 MHz, CDCl₃) δ ppm 9.11 (s, 1H), 7.61-7.49 (m, 3H), 7.30-7.24 (m, 4H), 7.18-7.05 (m, 2H), 3.81-3.43 (m, 2H), 3.24-3.02 (m, 2H), 1.25 (t, J=6.3 Hz, 3H), 0.77 (t, J=6.4 Hz, 1H); ¹³C NMR (101 MHz, CDCl₃) 6 ppm 167.82, 135.79, 134.70, 131.62, 128.75 (2C), 128.08, 127.47, 126.90 (2C), 122.72, 120.55, 119.33, 111.17, 109.74, 43.13, 38.99, 14.03, 12.74. MS EI m/z (rel. int.) 292 (M⁺, 25), 221 (61), 220 (100); HRMS m/z (EI, M⁺) calcd for C₁₉H₂₀N₂O, 292.1576. found 292.1582.

### N,N-Diethyl-2-(4-fluorophenyl)thiophene-3-carboxamide

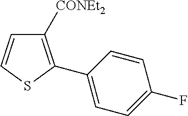

Light yellow solid. mp 62-63° C. (EtOAc/hexanes); IR (KBr) νₘₐₓ 2975, 2935, 1626, 1505, 1435, 1286, 1234, 1099, 839 cm⁻; ¹H NMR (400 MHz, CDCl₃) δ ppm 7.56-7.44 (m, 2H), 7.29 (d, J=5.2 Hz, 1H), 7.10-6.98 (m, 3H), 3.48 (q, J=7.1 Hz, 2H), 2.99 (q, J=7.1 Hz, 2H), 1.12 (t, J=7.1 Hz, 3H), 0.79 (t, J=7.1 Hz, 3H); ¹³C NMR (101 MHz, CDCl₃) δ ppm 167.36, 162.63 (d, ¹J_{C-F}=248.5 Hz), 138.84, 133.53, 129.70 (d, ³J_{C-F}=8.1 Hz, 2C), 129.44 (d, =3.3 Hz), 127.68, 125.18, 115.76 (d, ²J_{C-F}=21.7 Hz, 2C), 42.75, 39.01, 13.78, 12.3. MS EI m/z (rel. int.) 277 (M⁺, 24), 244 (12), 205 (100), 133 (25); HRMS m/z (EI, M⁺) calcd for C₁₅H₁₆FNOS, 277.0937. found 277.0934.

### N,N-Diethyl-3-(4-fluorophenyl)furan-2-carboxamide

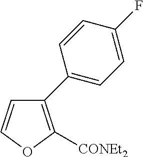

Light yellow oil. IR (KBr) νₘₐₓ 2977, 1634, 1516, 1433, 1223, 1158, 856, 839 cm⁻; ¹H NMR (400 MHz, CDCl₃) δ ppm 7.54-7.46 (m, 2H), 7.44 (d, J=1.8 Hz, 1H), 7.10-7.01 (m, 2H), 6.60 (d, J=1.8 Hz, 1H), 3.59-3.40 (m, 2H), 3.25-3.12 (m, 2H), 1.23-1.16 (m, 3H), 1.11-0.98 (m, 3H); ¹³C NMR (101 MHz, CDCl₃) δ ppm 162.28 (d, =247.1 Hz), 161.79, 142.80, 142.26, 129.49 (d, ³J_{C-F}=8.0 Hz, 2C), 128.04 (d, ⁴J_{C-F}=3.4 Hz), 125.47, 115.48 (d, ²J_{C-F}=21.5 Hz, 2C), 111.43, 43.01, 39.83, 14.25, 12.54. MS EI m/z (rel. int.) 261 (M⁺, 27), 190 (30), 189 (100), 162 (14), 133 (17); HRMS m/z (EI, M⁺) calcd for C₁₅H₁₆FNO₂, 261.1165. found 261.1166.

### N,N-Diethyl-2-phenylfuran-3-carboxamide

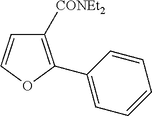

Light yellow oil. IR (KBr) νₘₐₓ 2974, 2935, 1631, 1491, 1430, 1295, 1216, 1061, 775, 758, 692 cm⁻¹; ¹H NMR (400 MHz, CDCl₃) δ ppm 7.66 (d, J=7.3 Hz, 2H), 7.46 (d, J=1.8 Hz, 1H), 7.37 (t, J=7.5 Hz, 2H), 7.32-7.25 (m, 1H), 6.49 (d, J=1.8 Hz, 1H), 3.58 (q, J=7.1 Hz, 2H), 3.20 (q, J=7.1 Hz, 2H), 1.26 (t, J=7.1 Hz, 3H), 0.95 (t, J=7.1 Hz, 3H); ¹³C NMR (101 MHz, CDCl₃) δ ppm 166.21, 149.26, 141.62, 130.00, 128.59 (2C), 128.03, 125.06 (2C), 116.90, 111.59, 43.03, 39.17, 14.05, 12.53. MS EI m/z (rel. int.) 243 (M⁺, 25), 214 (10), 171 (100), 115 (10); HRMS m/z (EI, M⁺) calcd for C₁₅H₁₇NO₂, 243.1259. found 243.1261.

### N,N-Diethyl-2-(p-tolyl)furan-3-carboxamide

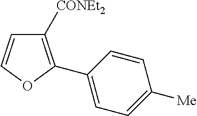

Light yellow oil. IR (KBr) νₘₐₓ 2973, 1934, 1630, 1496, 1429, 1294, 1069, 821 cm⁻; ¹H NMR (400 MHz, CDCl₃) δ ppm 7.54 (d, J=8.2 Hz, 2H), 7.42 (d, J=1.8 Hz, 1H), 7.17 (d, J=8.0 Hz, 2H), 6.47 (d, J=1.8 Hz, 1H), 3.57 (q, J=7.1 Hz, 2H), 3.19 (q, J=7.1 Hz, 2H), 2.34 (s, 3H), 1.25 (t, J=7.1 Hz, 3H), 0.94 (t, J=7.1 Hz, 3H); ¹³C NMR (101 MHz, CDCl₃) δ ppm 166.32, 149.49, 141.26, 137.97, 129.28 (2C), 127.29, 125.01 (2C), 116.16, 111.51, 43.00, 39.13, 21.23, 14.06, 12.52. MS EI m/z (rel. int.) 257 (M⁺, 35), 228 (10), 185 (100); HRMS m/z (EI, M⁺) calcd for C₁₆H₁₉NO₂, 257.1416. found 257.1417.

### N,N-Diethyl-2-(3-(t-butoxymethyl)phenyl)furan-3-carboxamide

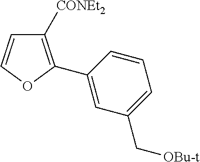

Light yellow oil. IR (KBr) νₘₐₓ 2974, 1633, 1482, 1459, 1431, 1363, 1194, 1064, 794 cm⁻; ¹H NMR (400 MHz, CDCl₃) δ ppm 7.62 (s, 1H), 7.54 (d, J=7.2 Hz, 1H), 7.44 (d, J=1.7 Hz, 1H), 7.37-7.27 (m, 2H), 6.48 (d, J=1.7 Hz, 1H), 4.44 (s, 2H), 3.57 (q, J=7.1 Hz, 2H), 3.18 (q, J=7.1 Hz, 2H), 1.29 (s, 9H), 1.25 (t, J=7.1 Hz, 3H), 0.94 (t, J=7.1 Hz, 3H); ¹³C NMR (101 MHz, CDCl₃) δ ppm 166.22, 149.26, 141.56, 140.37, 129.93, 128.63, 127.20, 124.05, 123.91, 116.81, 111.62, 73.49, 63.96, 43.05, 39.17, 27.65 (3C), 14.09, 12.59. MS EI m/z (rel. int.) 329 (M⁺, 100), 257 (26), 201 (64), 199 (27), 185 (65), 184 (45), 183 (77), 92 (24), 57 (24); HRMS m/z (EI, M⁺) calcd for C₂₀H₂₇NO₃, 329.1991. found 329.1988.

### N,N-Diethyl-2-(4-trifluoromethylphenyl)furan-3-carboxamide

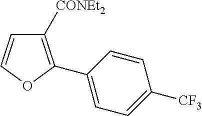

Light yellow solid. mp 45-48° C. (EtOAc/hexanes); IR (KBr) νₘₐₓ 2977, 2937, 1634, 1621, 1497, 1432, 1326, 1294, 1167, 1125, 1067, 846 cm⁻¹; ¹H NMR (400 MHz, CDCl₃) δ ppm 7.78 (d, J=8.1 Hz, 2H), 7.61 (d, J=8.3 Hz, 2H), 7.50 (d, J=1.8 Hz, 1H), 6.51 (d, J=1.8 Hz, 1H), 3.58 (q, J=7.1 Hz, 2H), 3.22 (q, J=7.1 Hz, 2H), 1.27 (t, J=7.1 Hz, 3H), 0.98 (t, J=7.1 Hz, 3H); ¹³C NMR (101 MHz, CDCl₃) δ ppm 165.69, 147.83, 142.55, 133.13, 129.63 (q, ²J_{C-F}=32.6 Hz), 125.63 (q, ³J_{C-F}=3.8 Hz, 2C), 125.05 (2C), 123.98 (q, ¹J_{C-F}=272.0 Hz), 118.84, 111.78, 43.11, 39.32, 14.16, 12.59. MS EI m/z (rel. int.) 311 (M⁺, 22), 282 (15), 239 (100); HRMS m/z (EI, M⁺) calcd for C₁₆H₁₆F₃NO₂, 311.1133. found 311.1131.

### N,N-Diethyl-2-(4-(dimethylamino)phenyl)furan-3-carboxamide

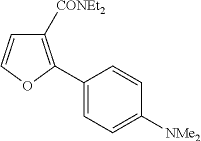

Light yellow oil. mp 73-74° C. (EtOAc/hexanes); IR (KBr) νₘₐₓ 1625, 1618, 1528, 1500, 1429, 1362, 1199, 1065, 820 cm⁻¹; ¹H NMR (400 MHz, CDCl₃) δ ppm 7.53 (d, J=8.9 Hz, 2H), 7.36 (d, J=1.8 Hz, 1H), 6.69 (d, J=8.9 Hz, 2H), 6.44 (d, J=1.8 Hz, 1H), 3.56 (q, J=7.1 Hz, 2H), 3.20 (q, J=7.1 Hz, 2H), 2.97 (s, 6H), 1.25 (t, J=7.0 Hz, 3H), 0.95 (t, J=7.0 Hz, 3H); ¹³C NMR (101 MHz, CDCl₃) δ ppm 166.75, 150.39, 150.05, 140.27, 126.31 (2C), 118.45, 113.91, 111.95, 111.48 (2C), 42.98, 40.23 (2C), 39.11, 14.09, 12.61. MS EI m/z (rel. int.) 286 (M⁺, 80), 214 (100), 158 (23), 106 (18); HRMS m/z (EI, M⁺) calcd for C₁₇H₂₂N₂O₂, 286.1681. found 286.1680.

### N,N-Diethyl-2-(3-methoxyphenyl)furan-3-carboxamide

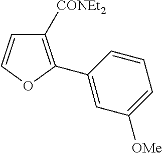

Light yellow oil. IR (KBr) νₘₐₓ 2974, 2936, 1630, 1578, 1492, 1460, 1433, 1293, 1271, 1220, 1043, 786 cm⁻; ¹H NMR (400 MHz, CDCl₃) δ ppm 7.45 (d, J=1.8 Hz, 1H), 7.32-7.17 (m, 3H), 6.89-6.78 (m, 1H), 6.49 (d, J=1.8 Hz, 1H), 3.82 (s, 3H), 3.57 (q, J=7.1 Hz, 2H), 3.21 (q, J=7.1 Hz, 2H), 1.26 (t, J=7.1 Hz, 3H), 0.96 (t, J=7.1 Hz, 3H); ¹³C NMR (101 MHz, CDCl₃) δ ppm 166.18, 159.78, 149.09, 141.60, 131.22, 129.66, 117.60, 117.13, 114.21, 111.61, 110.18, 55.23, 43.08, 39.25, 14.08, 12.63. MS EI m/z (rel. int.) 273 (M⁺, 38), 202 (58), 201 (100), 174 (14); HRMS m/z (EI, M⁺) calcd for C₁₆H₁₉NO₃, 273.1365. found 273.1362.

### N,N-Diethyl-2-(4-methoxyphenyl)furan-3-carboxamide

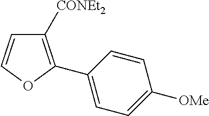

Light yellow oil. IR (KBr) νₘₐₓ 2973, 2935, 1629, 1599, 1520, 1497, 1460, 1431, 1296, 1254, 1180, 1068, 1033, 835 cm⁻; ¹H NMR (400 MHz, CDCl₃) δ ppm 7.59 (d, J=8.9 Hz, 2H), 7.39 (d, J=1.8 Hz, 1H), 6.89 (d, J=8.9 Hz, 2H), 6.45 (d, J=1.8 Hz, 1H), 3.81 (s, 3H), 3.56 (q, J=7.0 Hz, 2H), 3.19 (q, J=7.0 Hz, 2H), 1.24 (t, J=7.1 Hz, 3H), 0.94 (t, J=7.1 Hz, 3H); ¹³C NMR (101 MHz, CDCl₃) δ ppm 166.41, 159.47, 149.53, 140.95, 126.64 (2C), 122.98, 115.38, 114.03 (2C), 111.48, 55.22, 43.02, 39.16, 14.08, 12.58. MS EI m/z (rel. int.) 273 (M⁺, 38), 201 (100); HRMS m/z (EI, M⁺) calcd for C₁₆H₁₉NO₃, 273.1365. found 273.1360.

### N,N-Diethyl-2-(2-fluorophenyl)furan-3-carboxamide

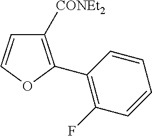

Light yellow oil. IR (KBr) νₘₐₓ 2975, 2936, 1632, 1598, 1494, 1457, 1430, 1294, 1220, 1064, 758 cm⁻¹; ¹H NMR (400 MHz, CDCl₃) δ ppm 7.65 (td, J=7.6, 1.6 Hz, 1H), 7.51 (d, J=1.8 Hz, 1H), 7.34-7.27 (m, 1H), 7.17 (td, J=7.6, 1.0 Hz, 1H), 7.13-7.04 (m, 1H), 6.53 (d, J=1.8 Hz, 1H), 3.51 (q, J=7.1 Hz, 2H), 3.26 (q, J=7.1 Hz, 2H), 1.20 (t, J=7.1 Hz, 3H), 1.00 (t, J=7.1 Hz, 3H); ¹³C NMR (101 MHz, CDCl₃) 6 ppm 165.63, 158.82 (d, ¹J_{C-F}=251.5 Hz), 145.48 (d, ⁴J_{C-F}=1.9 Hz), 142.23, 130.04 (d, ³J_{C-F}=8.3 Hz), 128.94 (d, ⁴J_{C-F}=2.8 Hz), 124.23 (d, ³J_{C-F}=3.5 Hz), 119.81 (d, ³J_{C-F}=2.1 Hz), 118.18 (d, ²J_{C-F}=13.5 Hz), 116.10 (d, ²J_{C-F}=21.8 Hz), 111.47, 42.84, 38.91, 13.86, 12.42. MS EI m/z (rel. int.) 261 (M⁺, 30), 232 (15), 190 (15), 189 (100); HRMS m/z (EI, M⁺) calcd for C₁₅H₁₆FNO₂, 261.1165. found 261.1167.

### N,N-Diethyl-2-(4-fluorophenyl)furan-3-carboxamide

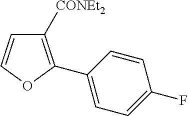

Light yellow oil. IR (KBr) νₘₐₓ 2975, 2936, 1630, 1601, 1518, 1496, 1460, 1431, 1295, 1234, 1159, 1068, 839,755 cm⁻¹; ¹H NMR (400 MHz, CDCl₃) δ ppm 7.70-7.58 (m, 2H), 7.43 (d, J=1.8 Hz, 1H), 7.11-6.98 (m, 2H), 6.47 (d, J=1.8 Hz, 1H), 3.56 (q, J=7.0 Hz, 2H), 3.20 (q, J=7.0 Hz, 2H), 1.24 (t, J=7.0 Hz, 3H), 0.95 (t, J=7.0 Hz, 3H); ¹³C NMR (101 MHz, CDCl₃) δ ppm 166.07, 162.45 (d, ¹J_{C-F}=248.3 Hz), 148.63, 141.56, 127.04 (d, ³J_{C-F}=8.1 Hz, 2C), 126.36 (d, ⁴J_{C-F}=3.3 Hz), 116.65, 115.68 (d, ²J_{C-F}=21.8 Hz, 2C), 111.53, 43.06, 39.23, 14.11, 12.59. MS EI m/z (rel. int.) 261 (M⁺, 27), 232 (11), 189 (100), 133 (10); HRMS m/z (EI, M⁺) calcd for C₁₅H₁₆FNO₂, 261.1165. found 261.1160.

### N,N-Diethyl-2-(2,3-dimethylphenyl)furan-3-carboxamide

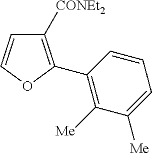

Light yellow oil. IR (KBr) νₘₐₓ 2973, 2935, 1631, 1478, 1458, 1433, 1062, 788 cm⁻¹; ¹H NMR (400 MHz, CDCl₃) δ ppm 7.48 (d, J=1.8 Hz, 1H), 7.21 (d, J=7.6 Hz, 1H), 7.17 (d, J=7.3 Hz, 1H), 7.09 (t, J=7.5 Hz, 1H), 6.57 (d, J=1.8 Hz, 1H), 3.42 (q, J=7.0 Hz, 2H), 3.10 (q, J=7.0 Hz, 2H), 2.31 (s, 3H), 2.22 (s, 3H), 1.08 (t, J=6.9 Hz, 3H), 0.76 (t, J=6.9 Hz, 3H); ¹³C NMR (101 MHz, CDCl₃) δ ppm 165.72, 151.58, 141.68, 137.40, 135.57, 130.72, 129.87, 127.96, 125.42, 118.74, 111.23, 42.90, 38.91, 20.46, 16.76, 13.61, 12.49. MS EI m/z (rel. int.) 271 (M⁺, 4), 199 (100), 198 (50), 171 (22), 143 (14), 128 (23), 72 (16); HRMS m/z (EI, M⁺) calcd for C₁₇H₂₁NO₂, 271.1572. found 271.1567.

### N,N-Diethyl-2-(3,5-difluorophenyl)furan-3-carboxamide

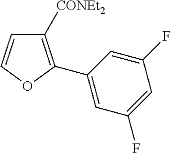

Light yellow oil. IR (KBr) νₘₐₓ 2976, 2937, 1626, 1583, 1506, 1481, 1432, 1321, 1290, 1216, 1121, 1083, 983, 866, 823 cm⁻; ¹H NMR (400 MHz, CDCl₃) δ ppm 7.47 (d, J=1.8 Hz, 1H), 7.24-7.14 (m, 2H), 6.72 (tt, J=8.7, 2.3 Hz, 1H), 6.50 (d, J=1.8 Hz, 1H), 3.59 (q, J=7.1 Hz, 2H), 3.22 (q, J=7.1 Hz, 2H), 1.28 (t, J=7.1 Hz, 3H), 1.00 (t, J=7.1 Hz, 3H); ¹³C NMR (101 MHz, CDCl₃) δ ppm 165.43, 163.22 (dd, ^{1,3}J_{C-F}=247.8, 13.0 Hz, 2C), 147.01 (t, ⁴J_{C-F}=3.6 Hz), 142.44, 132.63 (t, ³J_{C-F}=10.6 Hz), 118.89, 111.76, 107.68 (dd, ^{2,4}J_{C-F}=27.7, 8.0 Hz, 2C), 103.23 (t, ²J_{C-F}=25.5 Hz), 43.10, 39.35, 14.16, 12.48. MS EI m/z (rel. int.) 279 (M⁺, 24), 250 (10), 207 (100), 151 (12); HRMS m/z (EI, M⁺) calcd for C₁₅H₁₅F₂NO₂, 279.1071. found 279.1064.

### N,N-Diethyl-2-(naphthalen-2-yl)furan-3-carboxamide

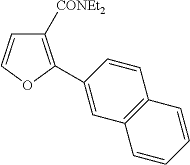

Pale solid. mp 92-93° C. (EtOAc/hexanes); IR (KBr) νₘₐₓ 2973, 1627, 1478, 1430, 1294, 832, 744 cm⁻¹; ¹H NMR (400 MHz, CDCl₃) δ ppm 8.14 (s, 1H), 7.91-7.73 (m, 4H), 7.51 (d, J=1.6 Hz, 1H), 7.50-7.39 (m, 2H), 6.55 (d, J=1.6 Hz, 1H), 3.62 (q, J=7.0 Hz, 2H), 3.21 (q, J=7.0 Hz, 2H), 1.32 (t, J=7.0 Hz, 3H), 0.94 (t, J=7.0 Hz, 3H); ¹³C NMR (101 MHz, CDCl₃) δ ppm 166.26, 149.24, 141.88, 133.27, 132.84, 128.35, 128.27, 127.65, 127.42, 126.46, 126.33, 124.09, 122.86, 117.35, 111.81, 43.09, 39.28, 14.09, 12.62. MS EI m/z (rel. int.) 293 (M⁺, 35), 222 (64), 221 (100), 165 (28); HRMS m/z (EI, M⁺) calcd for C₁₉H₁₉NO₂, 293.1416. found 293.1417.

### N,N-Diethyl-2-(furan-2-yl)furan-3-carboxamide

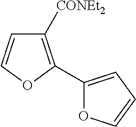

Light yellow oil. IR (KBr) νₘₐₓ 2975, 1629, 1487, 1462, 1430, 1293, 1068, 1008, 740 cm⁻¹; ¹H NMR (400 MHz, CDCl₃) δ ppm 7.42 (d, J=1.7 Hz, 1H), 7.39 (d, J=1.7 Hz, 1H), 6.64 (d, J=3.4 Hz, 1H), 6.48 (d, J=1.8 Hz, 1H), 6.44 (dd, J=3.3, 1.8 Hz, 1H), 3.64-3.50 (m, 2H), 3.35-3.16 (m, 2H), 1.26 (t, J=6.4 Hz, 3H), 1.01 (t, J=6.5 Hz, 3H); ¹³C NMR (101 MHz, CDCl₃) δ ppm 164.98, 145.07, 142.57, 142.51, 141.54, 116.35, 111.47, 111.11, 107.54, 43.02, 39.17, 14.08, 12.68. MS EI m/z (rel. int.) 233 (M⁺, 28), 161 (100), 105 (20); HRMS m/z (ESI, [M+1]⁺) calcd for C₁₃H₁₆NO₃, 234.1130. found 234.1126.

### N,N-Diethyl-2-(thiophen-3-yl)furan-3-carboxamide

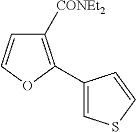

Light yellow oil. IR (KBr) νₘₐₓ 2974, 1627, 1492, 1435, 1291, 1067, 790 cm⁻¹; ¹H NMR (400 MHz, CDCl₃) δ ppm 7.59 (dd, J=2.9, 1.2 Hz, 1H), 7.38 (d, J=1.8 Hz, 1H), 7.37 (dd, J=5.9, 1.2 Hz, 1H), 7.31 (dd, J=5.1, 3.0 Hz, 1H), 6.45 (d, J=1.8 Hz, 1H), 3.64-3.44 (m, 2H), 3.35-3.17 (m, 2H), 1.26 (t, J=6.9 Hz, 3H), 1.00 (t, J=6.9 Hz, 3H); ¹³C NMR (101 MHz, CDCl₃) δ ppm 165.95, 147.27, 140.83, 131.15, 126.00, 125.11, 121.35, 115.86, 111.01, 43.11, 39.29, 14.20, 12.77. MS EI m/z (rel. int.) 249 (M⁺, 33), 178 (42), 177 (100), 121 (33); HRMS m/z (EI, M⁺) calcd for C₁₃H₁₅NO₂S, 249.0824. found 249.0814.

### N,N-Diethyl-2-(benzofuran-2-yl)furan-3-carboxamide

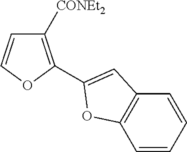

Light yellow oil. IR (KBr) νₘₐₓ 2974, 1630, 1493, 1455, 1430, 1254, 1076, 750 cm⁻¹; ¹H NMR (400 MHz, CDCl₃) δ ppm 7.58 (d, J=7.2 Hz, 1H), 7.50 (d, J=1.7 Hz, 1H), 7.45 (d, J=7.9 Hz, 1H), 7.33-7.18 (m, 2H), 7.02 (s, 1H), 6.56 (d, J=1.7 Hz, 1H), 3.70-3.56 (m, 2H), 3.37-3.21 (m, 2H), 1.36 (t, J=6.9 Hz, 3H), 1.03 (t, J=6.9 Hz, 3H); ¹³C NMR (101 MHz, CDCl₃) δ ppm 164.71, 154.68, 146.58, 142.75, 141.97, 128.31, 124.81, 123.25, 121.28, 118.81, 111.48, 111.19, 103.41, 43.12, 39.26, 14.12, 12.70. MS EI m/z (rel. int.) 283 (M⁺, 27), 212 (30), 211 (100), 155 (72), 126 (20), 57 (29), 56 (29); HRMS m/z (EI, M⁺) calcd for C₁₇H₁₇NO₃, 283.1208. found 283.1221.

### N,N-Diethyl-2-(4-formylphenyl)furan-3-carboxamide

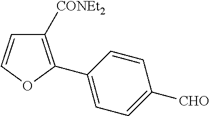

Light yellow solid. mp 64-66° C. (EtOAc/hexanes); IR (KBr) νₘₐₓ 2974, 1699, 1628, 1608, 1493, 1432, 1309, 1294, 1214, 1172, 1070, 832 cm⁻¹; ¹H NMR (400 MHz, CDCl₃) δ ppm 10.00 (s, 1H), 7.89 (d, J=8.6 Hz, 2H), 7.84 (d, J=8.5 Hz, 2H), 7.54 (d, J=1.8 Hz, 1H), 6.54 (d, J=1.8 Hz, 1H), 3.61 (q, J=7.1 Hz, 2H), 3.23 (q, J=7.1 Hz, 2H), 1.29 (t, J=7.1 Hz, 3H), 0.99 (t, J=7.1 Hz, 3H); ¹³C NMR (101 MHz, CDCl₃) δ ppm 191.49, 165.67, 147.88, 143.01, 135.32, 135.25, 130.16 (2C), 125.18 (2C), 119.74, 112.03, 43.14, 39.36, 14.19, 12.60. MS EI m/z (rel. int.) 271 (M⁺, 2), 199 (20), 171 (26), 115 (100), 56 (32); HRMS m/z (EI, M⁺) calcd for C₁₆H₁₇NO₃, 271.1208. found 271.1215.

### N,N-Diethyl-2-(4-chlorophenyl)furan-3-carboxamide

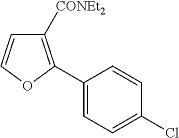

Light yellow solid (with 63% recovery of N,N-diethylfuran-3-carboxamide). mp 64-66° C. (EtOAc/hexanes); IR (KBr) νₘₐₓ 2975, 1630, 1489, 1431, 1295, 1094, 1068, 832 cm⁻¹; ¹H NMR (400 MHz, CDCl₃) δ ppm 7.61 (d, J=8.6 Hz, 2H), 7.45 (d, J=1.8 Hz, 1H), 7.34 (d, J=8.6 Hz, 2H), 6.49 (d, J=1.8 Hz, 1H), 3.57 (q, J=7.0 Hz, 2H), 3.20 (q, J=7.0 Hz, 2H), 1.25 (t, J=7.1 Hz, 3H), 0.97 (t, J=7, 1 Hz, 3H); ¹³C NMR (101 MHz, CDCl₃) δ ppm 165.97, 148.35, 141.88, 133.89, 128.89 (2C), 128.49, 126.33 (2C), 117.38, 111.66, 43.09, 39.27, 14.17, 12.61. MS EI m/z (rel. int.) 277 (M⁺, 28), 248 (19), 207 (30), 205 (100), 170 (15), 149 (14); HRMS m/z (EI, M⁺) calcd for C₁₅H₁₆ClNO₂, 277.0870. found 277.0869.

### N,N-Diethyl-2-(2-phenylcyclopropyl)furan-3-carboxamide

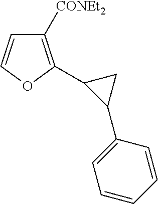

Light yellow oil, IR (KBr) νₘₐₓ 2973, 2934, 1623, 1496, 1477, 1459, 1433, 1380, 1297, 1215, 1138, 1055, 752, 735, 698 cm⁻¹; ¹H NMR (400 MHz, CDCl₃) δ ppm 7.27 (t, J=7.4 Hz, 2H), 7.20 (d, J=1.9 Hz, 1H), 7.17 (t, J=7.4 Hz, 1H), 7.13 (d, J=7.2 Hz, 2H), 6.36 (d, J=1.9 Hz, 1H), 3.55-3.27 (m, 4H), 2.48 (dt, J=8.8, 5.3 Hz, 1H), 2.42 (dt, J=9.0, 5.3 Hz, 1H), 1.61 (ddd, J=8.9, 5.6, 5.0 Hz, 1H), 1.40 (ddd, J=9.0, 6.0, 5.0 Hz, 1H), 1.22-1.04 (m, 6H); ¹³C NMR (101 MHz, CDCl₃) δ ppm 165.74, 154.72, 141.24, 139.49, 128.38 (2C), 125.97 (3C), 116.12, 110.18, 43.05 (br), 39.19 (br), 25.18, 20.25, 16.44, 14.12 (br), 13.05 (br). MS EI m/z (rel. int.) 283 (M⁺, 6), 192 (44), 153 (64), 152 (60), 128 (37), 115 (48), 104 (100), 103 (32), 91 (71), 78 (55), 77 (66), 56 (45), 51 (49); HRMS m/z (EI, M⁺) calcd for C₁₈H₂₁NO₂, 283.1572. found 283.1566.

### N,N-Diethyl-2-(3-t-butoxymethylphenyl)benzamide

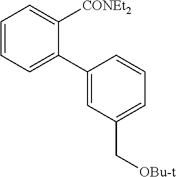

Light yellow oil. IR (KBr) νₘₐₓ 2973, 2933, 1630, 1470, 1459, 1431, 1363, 1290, 1195, 1090, 1071, 757 cm⁻¹; ¹H NMR (400 MHz, CDCl₃) δ ppm 7.46-7.39 (m, 3H), 7.38-7.31 (m, 5H), 4.46 (s, 2H), 3.81-3.65 (m, 1H), 3.07-2.90 (m, 2H), 2.74-2.58 (m, 1H), 1.28 (s, 9H), 0.90 (t, J=7.1 Hz, 3H), 0.74 (t, J=7.1 Hz, 3H); ¹³C NMR (101 MHz, CDCl₃) δ ppm 170.50, 140.00, 139.64, 138.43, 136.29, 129.46, 128.82, 128.24, 127.69, 127.58, 127.39, 126.93, 126.47, 73.41, 63.94, 42.33, 38.38, 27.64 (3C), 13.39, 11.99. MS EI m/z (rel. int.) 339 (M⁺, 15), 209 (24), 194 (45), 193 (100), 181 (48), 152 (30), 72 (39); HRMS m/z (EI, M⁺) calcd for C₂₂H₂₉NO₂, 339.2198. found 339.2205.

### N,N-Diethyl-2-((4-trifluoromethyl)phenyl)benzamide

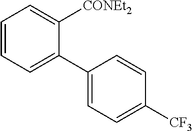

Light yellow solid. mp 81-82° C. (EtOAc/hexanes); IR (KBr) νₘₐₓ 2977, 1628, 1430, 1326, 1290, 1165, 1125, 1109, 1069, 767 cm⁻¹; ¹H NMR (400 MHz, CDCl₃) δ ppm 7.68-7.57 (m, 4H), 7.51-7.33 (m, 4H), 3.83-3.62 (m, 1H), 3.13-2.83 (m, 2H) 2.77-2.58 (m, 1H), 0.88 (t, J=7.1 Hz, 3H), 0.78 (t, J=7.1 Hz, 3H); ¹³C NMR (101 MHz, CDCl₃) δ ppm 169.99, 143.39, 136.88, 136.41, 129.70 (q, ²J_{C-F}=32.7 Hz), 129.36, 129.20 (2C), 129.08, 128.32, 126.96, 125.17 (q, ³J_{C-F}=3.7 Hz, 2C), 124.13 (q, ¹J_{C-F}=271.9 Hz), 42.29, 38.37, 13.42, 11.85. MS EI m/z (rel. int.) 321 (M⁺, 31), 320 (52), 249 (100), 201 (33), 152 (18); HRMS m/z (EI, M⁴) calcd for C₁₈H₁₈F₃NO, 321.1340. found 321.1334.

### N,N-Diethyl-2-(4-(dimethylamino)phenyl)benzamide

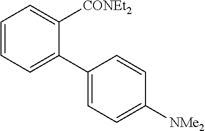

Light yellow oil. IR (KBr) νₘₐₓ 2973, 2933, 2875, 2830, 1625, 1613, 1527, 1484, 1443, 1429, 1356, 1288, 1223, 783 cm⁻¹; ¹H NMR (400 MHz, CDCl₃) δ ppm 7.42-7.26 (m, 6H), 6.72 (d, J=8.8 Hz, 2H), 3.81-3.64 (m, 1H), 3.15-3.02 (m, 1H), 3.00-2.87 (m, 711), 2.72-2.59 (m, 1H), 0.98 (t, J=7.1 Hz, 3H), 0.73 (t, J=7.1 Hz, 3H); ¹³C NMR (101 MHz, CDCl₃) δ ppm 171.05, 149.98, 138.45, 135.97, 129.49 (2C), 128.99, 128.74, 127.92, 127.05, 126.38, 112.21 (2C), 42.18, 40.46 (2C), 38.38, 13.33, 12.18. MS EI m/z (rel. int.) 296 (M⁺, 100), 295 (24), 224 (88); HRMS m/z (EI, M⁺) calcd for C₁₉H₂₄N₂O, 296.1889. found 296.1885.

### N,N-Diethyl-2-(3-methoxyphenyl)benzamide

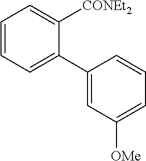

Light yellow oil. IR (KBr) νₘₐₓ 2972, 2935, 1627, 1602, 1581, 1464, 1429, 1318, 1291, 1221, 1094, 1053, 783, 761, 700 cm⁻¹; ¹H NMR (400 MHz, CDCl₃) δ ppm 7.46-7.32 (m, 4H), 7.27 (t, J=8.1 Hz, 1H), 7.10-6.99 (m, 2H), 6.87 (dd, J=8.2, 2.4 Hz, 1H), 3.81 (s, 3H), 3.78-3.68 (m, 1H), 3.09-2.89 (m, 2H), 2.74-2.59 (m, 1H), 0.90 (t, J=7.1 Hz, 3H), 0.75 (t, J=7.1 Hz, 3H); ¹³C NMR (101 MHz, CDCl₃) δ ppm 170.45, 159.35, 141.16, 138.23, 136.35, 129.26, 129.24, 128.82, 127.56, 126.91, 121.21, 114.11, 113.44, 55.22, 42.24, 38.26, 13.38, 11.92. MS EI m/z (rel. int.) 283 (M⁺, 46), 282 (45), 211 (100), 168 (18), 72 (17); HRMS m/z (EI, M⁺) calcd for C₁₈H₂₁NO₂, 283.1572. found 283.1574.

### N,N-Diethyl-2-(4-methoxyphenyl)benzamide

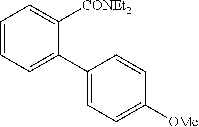

Light yellow oil. mp 46-47° C. (EtOAc/hexanes); IR (KBr) νₘₐₓ 2973, 2935, 1626, 1518, 1485, 1458, 1428, 1289, 1244, 1180, 1035, 836, 764 cm⁻¹; ¹H NMR (400 MHz, CDCl₃) δ ppm 7.46-7.29 (m, 6H), 6.90 (d, J=8.8 Hz, 2H), 3.81 (s, 3H), 3.78-3.66 (m, 1H), 3.10-2.86 (m, 2H), 2.71-2.59 (m, 1H), 0.93 (t, J=7.1 Hz, 3H), 0.73 (t, J=7.1 Hz, 3H); ¹³C NMR (101 MHz, CDCl₃) δ ppm 170.68, 159.16, 137.90, 136.20, 132.31, 129.94 (2C), 129.23, 128.82, 127.05, 126.94, 113.66 (2C), 55.25, 42.19, 38.33, 13.36, 12.08; MS EI m/z (rel. int.) 283 (M⁺, 36), 282 (30), 211 (100), 168 (19); FIRMS m/z (EI, M⁺) calcd for C₁₈H₂₁NO₂, 283.1572. found 283.1572.

### N,N-Diethyl-2-(2-fluorophenyl)benzamide

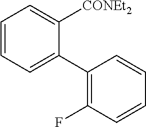

Light yellow on. IR (KBr) νₘₐₓ 2974, 2935, 1632, 1482, 1456, 1426, 1290, 1221, 1090, 757 cm⁻; ¹H NMR (400 MHz, CDCl₃) δ ppm 7.49-7.35 (m, 5H), 7.34-7.27 (m, 1H), 7.18-7.04 (m, 2H), 4.01-3.53 (m, 1H), 3.30-2.56 (m, 3H), 0.86 (t, J=7.1 Hz, 3H), 0.80 (t, J=7.1 Hz, 3H); ¹³C NMR (101 MHz, CDCl₃) δ ppm 169.85, 159.44 (d, ¹J_{C-F}=246.0 Hz), 137.19, 132.31, 132.09 (d, ⁴J_{C-F}=3.0 Hz), 130.63 (d, ⁴J_{C-F}=2.1 Hz), 129.43 (d, ³J_{C-F}=8.1 Hz), 128.34, 128.02, 127.09 (d, =15.0 Hz), 126.58, 123.85 (d, ³J_{C-F}=3.6 Hz), 115.34 (d, ²J_{C-F}=22.3 Hz), 42.14, 38.04, 13.50, 11.80. MS EI m/z (rel. int.) 271 (M⁺, 42), 270 (58), 199 (100), 170 (25); HRMS m/z (EI, M⁺) calcd for C₁₇H₁₈FNO, 271.1372. found 271.1368.

### N,N-Diethyl-2-(4-fluorophenyl)benzamide

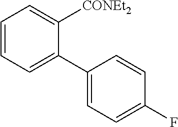

Light yellow solid. mp 57-59° C. (EtOAc/hexanes); IR (KBr) νₘₐₓ 2975, 2935, 1627, 1515, 1485, 1470, 1458, 1428, 1290, 1223, 1161, 1097, 840, 763 cm⁻; ¹H NMR (400 MHz, CDCl₃) δ ppm 7.50-7.30 (m, 6H), 7.05 (t, J=8.6 Hz, 2H), 3.83-3.63 (m, 1H), 3.12-2.84 (m, 2H), 2.75-2.57 (m, 1H), 0.91 (t, J=7.1 Hz, 3H), 0.75 (t, J=7.1 Hz, 31-1); ¹³C NMR (101 MHz, CDCl₃) δ ppm 170.31, 162.43 (d, F=247.0 Hz), 137.19, 136.32, 135.83 (d, ⁴J_{C-F}=3.3 Hz), 130.49 (d, ³J_{C-F}=8.0 Hz, 2C), 129.33, 128.91, 127.61, 126.87, 115.14 (d, ²J_{C-F}=21.4 Hz, 2C), 42.22, 38.32, 13.39, 12.00. MS EI m/z (rel. int.) 271 (M⁺, 24), 270 (50), 199 (100), 171 (18), 170 (28); FIRMS m/z (EI, M⁺) calcd for C₁₇H₁₈FNO, 271.1372. found 271.1382.

### N,N-Diethyl-2-(3,5-difluorophenyl)benzamide

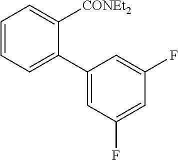

Light yellow oil. IR (KBr) νₘₐₓ 2976, 2935, 1625, 1592, 1433, 1414, 1338, 1292, 1120, 1093, 988, 864, 763 cm⁻¹; ¹H NMR (400 MHz, CDCl₃) δ ppm 7.49-7.32 (m, 4H), 7.07-6.96 (m, 2H), 6.78 (tt, J=8.9, 2.3 Hz, 1H), 3.98-3.66 (m, 1H), 3.16-2.86 (m, 2H), 2.84-2.65 (m, 1H), 0.97 (t, J=7.1 Hz, 314), 0.83 (t, J=7.1 Hz, 3H); ¹³C NMR (101 MHz, CDCl₃) δ ppm 169.78, 162.70 (dd, =248.7, 12.9 Hz, 2C), 142.93 (t, ³J_{C-F}=9.6 Hz), 136.29, 135.99 (t, ⁴J_{C-F}=2.3 Hz), 129.14, 129.11, 128.50, 127.00, 111.82 (dd, ^{2,4}J_{C-F}=25.8 Hz, 7.17 Hz, 2C), 102.85 (t, ²J_{C-F}=25.2 Hz), 42.39, 38.44, 13.49, 11.82. MS EI m/z (rel. int.) 289 (M⁺, 27), 288 (50), 217 (100), 189 (18), 188 (28); HRMS m/z (EI, M⁺) calcd for C₁₇H₁₇F₂NO, 289.1278. found 289.1278.

### N,N-Diethyl-2-(naphthalen-2-yl)benzamide

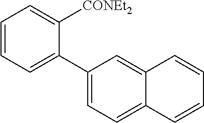

Light yellow solid. mp 52-53° C. (EtOAc/hexanes); IR (KBr) Vₘₐₓ 2974, 2933, 1625, 1474, 1458, 1424, 1290, 1089, 774, 761 cm⁻; ¹H NMR (400 MHz, CDCl₃) δ ppm 7.96 (s, 1H), 7.90-7.79 (m, 3H), 7.63 (dd, J=8.5, 1.8 Hz, 1H), 7.55-7.45 (m, 4H), 7.44-7.39 (m, 2H), 3.82-3.58 (m, 1H), 3.09-2.84 (m, 2H), 2.71-2.52 (m, 1H), 0.80 (t, J=7.1 Hz, 3H), 0.71 (t, J=7.1 Hz, 3H); ¹³C NMR (101 MHz, CDCl₃) δ ppm 170.57, 138.19, 137.21, 136.52, 133.15, 132.54, 129.70, 128.98, 128.21, 127.92, 127.74, 127.60, 127.54, 127.16, 126.96, 126.19, 126.07, 42.36, 38.47, 13.41, 12.00. MS EI m/z (rel. int.) 303 (M⁺, 30), 232 (48), 231 (100), 203 (21), 202 (54), 72 (21); HRMS m/z (EI, M⁺) calcd for C₂₁H₂₁NO, 303.1623. found 303.1624.

### N,N-Diethyl-2-(furan-2-yl)benzamide

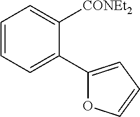

Yellow oil. IR (KBr) νₘₐₓ 2974, 2935, 1631, 1460, 1428, 1381, 1292, 1272, 1222, 1094, 1011, 761 cm⁻; ¹H NMR (400 MHz, CDCl₃) δ ppm 7.71 (dd, J=7.9, 0.6 Hz, 1H), 7.44 (dd, J=1.7, 0.6 Hz, 1H), 7.38 (td, J=7.9, 1.6 Hz, 1H), 7.29 (td, J=7.4, 1.2 Hz, 114), 7.24 (dd, J=7.5, 1.1 Hz, 1H), 6.64 (dd, J=3.4, 0.6 Hz, 114), 6.42 (dd, J=3.4, 1.8 Hz, 1H), 3.75 (q, J=7.0 Hz, 1H), 3.38 (q, J=7.0 Hz, 1H), 3.12-2.91 (m, 2H), 1.24 (t, J=7.1 Hz, 3H), 0.86 (t, J=7.1 Hz, 3H); ¹³C NMR (101 MHz, CDCl₃) 6 ppm 170.61, 151.61, 142.25, 133.85, 128.62, 127.47, 127.09, 126.81, 126.09, 111.64, 108.16, 42.59, 38.72, 13.34, 12.28. MS EI m/z (rel. int.) 243 (M⁺, 78), 171 (100), 143 (28), 115 (45); HRMS m/z (EI, M⁺) calcd for C₁₅H₁₇NO₂, 243.1259. found 243.1253.

### N,N-Diethyl-2-(thiophen-3-yl)benzamide

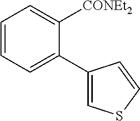

Yellow oil. IR (KBr) νₘₐₓ 2973, 2933, 1625, 1459, 1428, 1291, 1089, 860, 801, 774, 754 cm⁻; ¹H NMR (400 MHz, CDCl₃) δ ppm 7.51-7.29 (m, 6H), 7.27 (dd, J=5.0, 1.3 Hz, 1H), 3.81-3.66 (m, 1H), 3.22-3.08 (m, 1H), 3.02-2.87 (m, 1H), 2.82-2.68 (m, 1H), 1.04 (t, J=7.1 Hz, 3H), 0.75 (t, J=7.1 Hz, 1H); ¹³C NMR (101 MHz, CDCl₃) 6 ppm 170.68, 140.11, 136.08, 132.84, 128.86, 128.75, 128.19, 127.39, 126.80, 125.44, 123.17, 42.34, 38.48, 13.29, 12.18. MS EI m/z (rel. int.) 259 (M⁺, 29), 258 (15), 188 (36), 187 (100), 160 (19), 115 (48); HRMS m/z (EI, M⁺) calcd for C₁₅H₁₇NOS, 259.1031. found 259.1035.

### N,N-Diethyl-2-(benzofuran-2-yl)benzamide

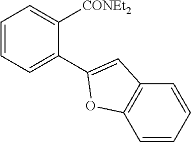

Light yellow oil. IR (KBr) νₘₐₓ 2974, 1632, 1491, 1472, 1455, 1427, 1290, 1258, 1088, 751 cm⁻; ¹H NMR (400 MHz, CDCl₃) δ ppm 7.92 (dd, J=7.8, 0.7 Hz, 1H), 7.56 (d, J=7.5 Hz, 1H), 7.51-7.43 (m, 2H), 7.40 (td, J=7.5, 1.2 Hz, 1H), 7.35-7.18 (m, 3H), 7.05 (s, 1H), 3.88-3.73 (m, 1H), 3.46-3.32 (m, 1H), 3.15-2.92 (m, 2H), 1.28 (t, J=7.1 Hz, 3H), 0.88 (t, J=7.1 Hz, 3H); ¹³C NMR (101 MHz, CDCl₃) δ ppm 170.44, 154.69, 153.51, 135.02, 129.01, 128.77, 128.58, 127.18, 126.98, 126.85, 124.52, 122.89, 121.19, 111.10, 104.76, 42.74, 38.87, 13.49, 12.41. MS EI m/z (rel. int.) 293 (M⁺, 66), 222 (47), 221 (100), 193 (17), 165 (36); HRMS m/z (EI, M⁺) calcd for C₁₉H₁₉NO₂, 293.1416. found 293.1416.

### (E)-N,N-Diethyl-2-styrylbenzamide

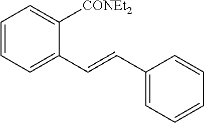

Light yellow oil. IR (KBr) νₘₐₓ 2973, 1628, 1598, 1495, 1485, 1469, 1458, 1449, 1428, 1381; 1285, 1075, 963, 762, 692 cm⁻; ¹H NMR (400 MHz, CDCl₃) δ ppm 7.70 (d, J=7.8 Hz, 1H), 7.46 (d, J=7.3 Hz, 2H), 7.40-7.18 (m, 6H), 7.13 (d, J=16.7 Hz, 1H), 7.09 (d, J=17.7 Hz, 1H), 4.05-3.68 (m, 1H), 3.56-3.22 (m, 1H), 3.10 (q, J=7.0 Hz, 2H), 1.30 (t, J=7.1 Hz, 3H), 1.00 (t, J=7.1 Hz, 3H); ¹³C NMR (101 MHz, CDCl₃) δ ppm 170.35, 137.01, 136.35, 133.63, 130.82, 128.75, 128.65 (2C), 127.84, 127.53, 126.56 (2C), 126.18, 125.25, 125.02, 42.82, 38.89, 13.87, 12.96. MS EI m/z (rel. int.) 279 (M⁺, 22), 208 (27), 207 (49), 179 (40), 178 (100), 177 (21), 176 (25), 152 (21), 77 (20), 57 (31), 56 (40); HRMS m/z (EI, M⁺) calcd for C₁₉H₂₁NO, 279.1623. found 279.1639.

### N,N-Diethyl-2-(2-phenylcyclopropyl)benzamide

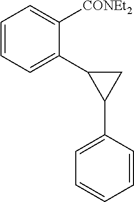

Yellow solid. mp 52-53° C. (EtOAc/hexanes); IR (KBr) νₘₐₓ 2973, 2933, 1631, 1602, 1494, 1472, 1459, 1428, 1291, 1072, 755, 698 cm⁻; ¹H NMR (400 MHz, CDCl₃) δ ppm 7.39-6.87 (in, 9H), 3.90-3.64 (m, 1H), 3.40-2.68 (m, 3H), 2.37-1.29 (m, 4H), 1.14-0.78 (m, 6H) (atropisomers involved); ¹³C NMR (101 MHz, CDCl₃) δ ppm 170.62, 142.11, 141.94, 137.89, 137.71, 128.75, 128.31, 128.24, 128.11, 126.16, 126.00, 125.75, 125.73, 125.54, 125.43, 125.18, 124.94, 123.04, 42.53, 42.45, 38.43, 28.83, 26.08, 25.15, 24.31, 17.25, 17.12, 13.90, 13.64, 12.45, 12.28 (atropisomers involved). MS EI m/z (rel. int.) 293 (M⁺, 2), 189 (100), 160 (29), 132 (13), 91 (14); HRMS m/z (EI, M⁺) calcd for C₂₀H₂₃NO, 293.1780. found 293.1780.

### 2-(Dimethylamino)-5-phenyl-N,N-diethylbenzamide

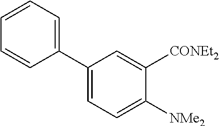

Light yellow oil. IR (KBr) νₘₐₓ 2973, 2936, 1625, 1515, 1486, 1458, 1432, 1378, 1320, 1263, 1137, 1081, 763, 699 cm⁻; ¹H NMR (400 MHz, CDCl₃) δ ppm 7.56 (d, J=7.3 Hz, 2H), 7.51 (dd, J=8.4, 2.0 Hz, 1H), 7.43 (d, J=2.0 Hz, 1H), 7.40 (t, J=7.6 Hz, 2H), 7.28 (t, J=7.4 Hz, 1H), 6.96 (d, J=8.4 Hz, 1H), 3.90-3.71 (m, 1H), 3.42-3.31 (m, 1H), 3.30-3.19 (m, 1H), 3.18-3.06 (m, 1H), 2.85 (s, 6H), 1.26 (t, J=7.1 Hz, 3H), 1.03 (t, J=7.1 Hz, 3H); ¹³C NMR (101 MHz, CDCl₃) δ ppm 171.25, 148.50, 140.22, 133.06, 129.52, 128.65 (2C), 127.89, 127.05, 126.64, 126.46 (2C), 117.15, 43.38 (2C), 42.75, 38.81, 13.75, 12.55. MS EI m/z (rel. int.) 296 (M⁺, 38), 224 (100), 223 (50), 196 (25), 181 (47), 180 (36), 167 (38), 153 (42), 152 (75), 72 (41), 58 (48), 57 (38), 56 (66); FIRMS m/z (ESI, [M+1]⁺) calcd for C₁₉H₂₅N₂O, 297.1966. found 297.1979.

### N,N-Diethyl-2-(4-methoxyphenyl)-5-phenylbenzamide

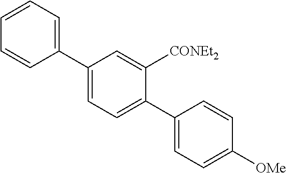

Pale solid. mp 139-141° C. (EtOAc/hexanes); IR (KBr) νₘₐₓ 2972, 2934, 1626, 1522, 1473, 1459, 1433, 1295, 1272, 1256, 1244, 1180, 1036, 829, 767, 700 cm⁻¹; ¹H NMR (400 MHz, CDCl₃) δ ppm 7.70-7.61 (m, 3H), 7.59 (d, J=1.3 Hz, 1H), 7.51-7.40 (m, 5H), 7.36 (t, J=7.2 Hz, 1H), 6.93 (d, J=8.5 Hz, 2H), 3.83 (s, 3H), 3.79-3.67 (m, 1H), 3.17-2.91 (m, 2H), 2.78-2.62 (m, 1H), 0.98 (t, J=7.1 Hz, 3H), 0.75 (t, J=7.1 Hz, 3H); ¹³C NMR (101 MHz, CDCl₃) δ ppm 170.66, 159.26, 139.99, 139.90, 136.85, 136.60, 131.92, 129.95 (2C), 129.74, 128.81 (2C), 127.52, 127.48, 126.96 (2C), 125.61, 113.76 (2C), 55.28, 42.32, 38.45, 13.46, 12.14. MS EI m/z (rel. int.) 359 (M⁺, 50), 358 (36), 288 (30), 287 (100), 216 (28), 215 (79), 77 (32), 72 (39), 57 (30), 56 (51); HRMS m/z (ESI, [M+1]⁺) calcd for C₂₄H₂₆NO₂, 360.1963. found 360.1979.

### 3-Methyl-2-phenyl-N,N-diethylbenzamide

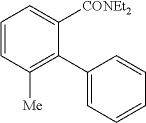

Light yellow oil. IR (KBr) νₘₐₓ 2973, 2932, 1633, 1478, 1456, 1441, 1426, 1330, 1315, 1291, 1122, 796, 773, 749, 703 cm⁻¹; ¹H NMR (400 MHz, CDCl₃) δ ppm 7.45-7.23 (m, 6H), 7.22-7.13 (m, 213), 3.86-3.67 (m, 1H), 3.20-3.02 (m, 1H), 2.82-2.61 (m, 213), 2.15 (s, 3H), 0.91 (t, J=7.1 Hz, 3H), 0.59 (t, J=7.1 Hz, 3H); ¹³C NMR (101 MHz, CDCl₃) δ ppm 170.22, 138.39, 137.78, 137.43, 136.37, 130.25, 128.60 (br), 128.37 (br), 127.46, 127.33 (br, 2C), 127.14, 123.17, 42.17, 37.57, 20.50, 13.57, 11.52. MS EI m/z (rel. int.) 267 (M⁺, 25), 266 (51), 195 (95), 166 (32), 165 (100), 152 (61), 56 (34); HRMS m/z (ESI, [M+1]⁺) calcd for C₁₈H₂₂NO, 268.1701. found 268.1692.

### N,N-Diethyl-5-methyl-2-(4-methoxyphenyl)benzamide

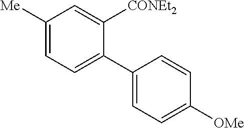

Light yellow solid. mp 113-114° C. (EtOAc/hexanes); IR (KBr) νₘₐₓ 2972, 2934, 1627, 1520, 1474, 1461, 1437, 1293, 1247, 1180, 1091, 1038, 821 cm⁻¹; ¹H NMR (400 MHz, CDCl₃) δ ppm 7.40 (d, J=8.8 Hz, 2H), 7.29-7.19 (m, 2H), 7.15 (s, 1H), 6.89 (d, J=8.8 Hz, 2H), 3.82 (s, 3H), 3.78-3.65 (m, 1H), 3.12-2.88 (m, 2H), 2.75-2.58 (m, 1H), 2.38 (s, 3H), 0.95 (t, J=7.1 Hz, 3H), 0.73 (t, J=7.1 Hz, 3H); ¹³C NMR (101 MHz, CDCl₃) δ ppm 170.88, 158.99, 136.88, 136.04, 135.03, 132.32, 129.88 (2C), 129.61, 129.13, 127.51, 113.63 (2C), 55.25, 42.20, 38.28, 20.94, 13.37, 12.12. MS EI m/z (rel. int.) 297 (M⁺, 32), 296 (29), 225 (100), 182 (20), 165 (16), 153 (24), 152 (17); HRMS m/z (ESI, [M+1]⁺) calcd for C₁₉H₂₄NO₂, 298.1807. found 298.1823.

### N,N-Diethyl-5-tert-butyl-2-(4-methoxyphenyl)benzamide

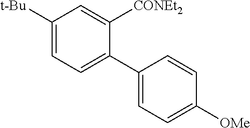

Light yellow solid. mp 89-92° C. (EtOAc/hexanes); IR (KBr) νₘₐₓ 2965, 1629, 1610, 1522, 1489, 1474, 1461, 1434, 1294, 1261, 1248, 1180, 1138, 828 cm⁻¹; ¹H NMR (400 MHz, CDCl₃) δ ppm 7.45-7.36 (m, 3H), 7.34 (d, J=2.0 Hz, 1H), 7.29 (d, J=8.1 Hz, 1H), 6.89 (d, J=8.7 Hz, 2H), 3.81 (s, 3H), 3.80-3.69 (m, 1H), 3.08-2.87 (m, 2H), 2.75-2.57 (m, 1H), 1.34 (s, 9H), 0.95 (t, J=7.1 Hz, 3H), 0.74 (t, J=7.1 Hz, 3H); ¹³C NMR (101 MHz, CDCl₃) δ ppm 171.22, 159.01, 150.00, 135.74, 135.01, 132.29, 129.89 (2C), 128.91, 125.90, 123.86, 113.64 (2C), 55.24, 42.22, 38.40, 34.53, 31.22, 13.38, 12.14. MS EI m/z (rel. int.) 339 (M⁺, 32), 267 (67), 211 (39), 165 (26), 72 (43), 57 (100); HRMS m/z (EI, M⁺) calcd for C₂₂H₂₉NO₂, 339.2198. found 339.2179.

### N,N-Diethyl-2,5-diphenylbenzamide

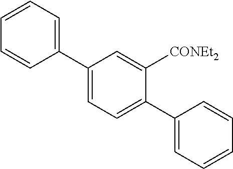

Light yellow solid. mp 128-130° C. (EtOAc/hexanes); IR (KBr) νₘₐₓ 2974, 2933, 1627, 1473, 1458, 1433, 1272, 1093, 758, 701 cm⁻¹; ¹H NMR (400 MHz, CDCl₃) δ ppm 7.74-7.63 (m, 3H), 7.62 (d, J=1.5 Hz, 1H), 7.54 (d, J=6.8 Hz, 2H), 7.51-7.43 (m, 3H), 7.43-7.30 (m, 4H), 3.87-3.70 (m, 1H), 3.13-2.90 (m, 2H), 2.79-2.61 (m, 1H), 0.93 (t, J=7.1 Hz, 3H), 0.74 (t, J=7.1 Hz, 3H); ¹³C NMR (101 MHz, CDCl₃) δ ppm 170.42, 140.37, 139.94, 139.38, 137.22, 136.77, 129.88, 128.83 (2C), 128.81 (2C), 128.32 (2C), 127.61, 127.57, 127.52, 127.01 (2C), 125.60, 42.27, 38.34, 13.42, 11.96. MS EI m/z (rel. int.) 329 (M⁺, 37), 328 (40), 257 (100), 228 (25); HRMS m/z (EI, M⁺) calcd for C₂₃H₂₃NO, 329.1780. found 329.1783.

### N,N-Dimethyl-2-methoxy-6-phenylbenzamide

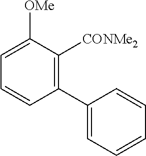

Light yellow oil. IR (KBr) νₘₐₓ 2935, 1639, 1593, 1583, 1570, 1500, 1466, 1429, 1394, 1309, 1270, 1256, 1123, 1098, 1059, 1019, 761, 702 cm⁻¹; ¹H NMR (400 MHz, CDCl₃) δ ppm 7.50-7.42 (m, 2H), 7.41-7.28 (m, 4H), 6.99 (dd, J=7.7, 0.6 Hz, 1H), 6.93 (d, J=8.3 Hz, 1H), 3.87 (s, 3H), 2.86 (s, 3H), 2.53 (s, 3H); ¹³C NMR (101 MHz, CDCl₃) δ ppm 168.59, 155.79, 140.21, 139.74, 129.62, 128.52 (2C), 128.17 (2C), 127.48, 125.06, 122.01, 109.84, 55.89, 37.63, 34.26. MS EI m/z (rel. int.) 255 (M⁺, 8), 211 (100), 168 (29), 152 (29), 139 (44), 72 (16); HRMS m/z (EI, M⁺) calcd for C₁₆H₁₇NO₂, 255.1259. found 255.1257.

### N,N-Diethyl-2-methoxy-6-phenylbenzamide

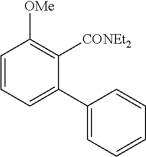

Pale solid. mp 79-80° C. (EtOAc/hexanes); IR (KBr) νₘₐₓ 2975, 2935, 1632, 1583, 1569, 1465, 1423, 1283, 1265, 761, 701 cm⁻; ¹H NMR (400 MHz, CDCl₃) δ ppm 7.48 (d, J=6.7 Hz, 2H), 7.41-7.27 (m, 4H), 6.97 (d, J=7.7 Hz, 1H), 6.92 (d, J=8.3 Hz, 1H), 3.85 (s, 3H), 3.84-3.73 (m, 1H), 3.05-2.87 (m, 2H), 2.77-2.63 (m, 1H), 0.84-0.72 (m, 6H); ¹³C NMR (101 MHz, CDCl₃) δ ppm 167.59, 155.75, 140.03, 139.61, 129.31, 128.92 (2C), 128.01 (2C), 127.38, 125.56, 121.96, 109.78, 55.69, 42.14, 37.88, 13.26, 11.85. MS EI m/z (rel. int.) 283 (M⁺, 8), 211 (100), 206 (18); HRMS m/z (EI, M⁺) calcd for C₁₈H₂₁NO₂, 283.1572. found 283.1570.

### N,N-Dimethyl-2-phenyl-3-methoxybenzamide

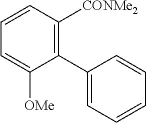

Light yellow solid. mp 83-84° C. (EtOAc/hexanes); IR (KBr) νₘₐₓ 2936, 1635, 1579, 1502, 1466, 1455, 1433, 1395, 1257, 1053, 701 cm⁻¹; ¹H NMR (400 MHz, CDCl₃) δ ppm 7.45-7.28 (m, 6H), 6.99 (dd, J=8.4, 2.3 Hz, 2H), 3.76 (s, 3H), 2.73 (s, 3H), 2.47 (s, 3H); ¹³C NMR (101 MHz, CDCl₃) δ ppm 170.63, 156.32, 138.19, 135.29, 129.98 (2C), 129.02, 127.64 (2C), 127.45, 127.34, 119.02, 111.55, 55.81, 38.05, 34.23. MS EI m/z (rel. int.) 255 (M⁺, 48), 211 (100), 196 (24); HRMS m/z (EI, M⁺) calcd for C₁₆H₁₇NO₂, 255.1259. found 255.1267.

### N,N-Diethyl-2-phenyl-3-methoxybenzamide

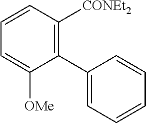

Light yellow solid. mp 79-80° C. (EtOAc/hexanes); IR (KBr) νₘₐₓ 2972, 2934, 1629, 1459, 1426, 1297, 1255, 1059, 801, 744, 700 cm⁻¹; ¹H NMR (400 MHz, CDCl₃) δ ppm 7.44-7.27 (m, 6H), 6.97 (t, J=7.9 Hz, 2H), 3.80-3.67 (m, 4H), 3.13-2.99 (m, 1H), 2.86-2.72 (m, 1H), 2.71-2.56 (m, 1H), 0.84 (t, J=7.1 Hz, 3H), 0.66 (t, J=7.1 Hz, 3H); ¹³C NMR (101 MHz, CDCl₃) δ ppm 169.70, 156.36, 138.62, 135.22, 130.16 (2C), 128.92, 127.64 (2C), 127.24, 127.20, 118.51, 111.22, 55.78, 42.04, 37.74, 13.50, 11.64. MS EI m/z (rel. int.) 283 (M⁺, 64), 282 (69), 212 (13), 211 (100), 196 (35), 168 (15); HRMS m/z (EI, M⁺) calcd for C₁₈H₂₁NO₂, 283.1572. found 283.1563.

### N,N-Diethyl-2-phenyl-4-methoxybenzamide

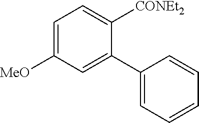

Light yellow solid. mp 64-65° C. (EtOAc/hexanes); IR (KBr) νₘₐₓ 2972, 2935, 1625, 1468, 1428, 1290, 1271, 1036, 772, 702 cm⁻¹; ¹H NMR (400 MHz, CDCl₃) δ ppm 7.47 (d, J=6.6 Hz, 2H), 7.40-7.27 (m, 4H), 6.96-6.85 (m, 2H), 3.84 (s, 3H), 3.79-3.63 (m, 1H), 3.16-2.78 (m, 2H), 2.73-2.48 (m, 1H), 0.86 (t, J=7.1 Hz, 3H), 0.72 (t, J=7.1 Hz, 3H); ¹³C NMR (101 MHz, CDCl₃) δ ppm 170.52, 159.70, 139.97, 139.76, 129.02, 128.70 (2C), 128.44, 128.24 (2C), 127.59, 114.62, 112.97, 55.33, 42.23, 38.29, 13.35, 11.90. MS EI m/z (rel. int.) 283 (M⁺, 11), 282 (16), 211 (100); HRMS m/z (EI, M⁺) calcd for C₁₈H₂₁NO₂, 283.1572. found 283.1574.

### N,N-Diethyl-2-phenyl-3-methoxylbenzamide

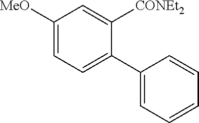

Light yellow solid. mp 55-56° C. (EtOAc/hexanes); IR (KBr) νₘₐₓ 2972, 2935, 1628, 1608, 1478, 1433, 1315, 1291, 1269, 1230, 1086, 1047, 830, 773, 704 cm⁻¹; ¹H NMR (400 MHz, CDCl₃) δ ppm 7.47-7.40 (m, 2H), 7.37-7.27 (m, 4H), 6.97 (dd, J=8.5, 2.7 Hz, 1H), 6.89 (d, J=2.6 Hz, 1H), 3.84 (s, 3H), 3.80-3.69 (m, 1H), 3.06-2.89 (m, 2H), 2.71-2.56 (m, 1H), 0.89 (t, J=7.1 Hz, 3H), 0.73 (t, J=7.1 Hz, 3H); ¹³C NMR (101 MHz, CDCl₃) δ ppm 170.21, 158.96, 139.47, 137.27, 130.88, 130.65, 128.77 (2C), 128.20 (2C), 127.05, 115.04, 111.93, 55.42, 42.17, 38.25, 13.35, 11.88; MS EI m/z (rel. int.) 283 (M⁺, 41), 282 (38), 211 (100), 168 (17); HRMS m/z (EI, M⁺) calcd for C₁₈H₂₁NO₂, 283.1572. found 283.1564.

### N,N-Diethyl-4-methoxymethoxy-2-phenylbenzamide

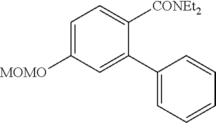

Light yellow solid. mp 63-64° C. (EtOAc/hexanes); IR (KBr) νₘₐₓ 2972, 2934, 1626, 1468, 1430, 1314, 1289, 1220, 1184, 1154, 1095, 1079, 996, 702 cm⁻¹; ¹H NMR (400 MHz, CDCl₃) δ ppm 7.47 (dd, J=8.0, 1.4 Hz, 2H), 7.39-7.27 (m, 4H), 7.08-7.02 (m, 2H), 5.21 (s, 2H), 3.85-3.63 (m, 1H), 3.49 (s, 3H), 3.15-2.84 (m, 2H), 2.74-2.49 (m, 1H), 0.88 (t, J=7.1 Hz, 3H), 0.72 (t, J=7.1 Hz, 3H); ¹³C NMR (101 MHz, CDCl₃) δ ppm 170.45, 157.44, 139.98, 139.60, 130.10, 128.72 (2C), 128.41, 128.25 (2C), 127.61, 116.96, 115.18, 94.40, 56.07, 42.25, 38.32, 13.36, 11.90. MS EI m/z (rel. int.) 313 (M⁺, 24), 312 (50), 241 (100), 211 (65), 168 (28), 139 (33); FIRMS m/z (ESI, [M+1]⁺) calcd for C₁₉H₂₄NO₃, 314.1756. found 314.1760.

### N,N-Dimethyl-2-phenyl-3,4-dimethoxybenzamide

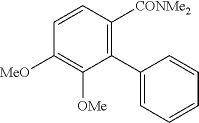

Colorless solid. mp 101-102° C. (EtOAc/hexanes); IR (KBr) νₘₐₓ 2936, 1633, 1596, 1479, 1450, 1394, 1296, 1273, 1258, 1122, 1021, 767, 701 cm⁻; ¹H NMR (400 MHz, CDCl₃) δ ppm 7.43 (d, J=6.8 Hz, 2H), 7.39-7.28 (m, 3H), 7.11 (d, J=8.4 Hz, 1H), 6.95 (d, J=8.4 Hz, 1H), 3.90 (s, 3H), 3.45 (s, 3H), 2.71 (s, 3H), 2.40 (s, 3H); ¹³C NMR (101 MHz, CDCl₃) δ ppm 170.65, 153.43, 146.17, 135.14, 133.25, 130.16, 129.77 (2C), 127.69 (2C), 127.46, 122.79, 111.72, 60.47, 55.91, 38.11, 34.34. MS EI m/z (rel. int.) 285 (M⁺, 39), 241 (100), 226 (47); HRMS m/z (EI, M⁺) calcd for C₁₇H₁₉NO₃, 285.1365. found 285.1360.

### 1-(tert-Butyldimethylsilyl)-N,N-diethyl-5-(4-methoxyphenyl)-1H-indole-4-carboxamide

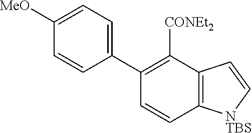

Light yellow oil. IR (KBr) νₘₐₓ 2957, 2932, 1626, 1521, 1464, 1424, 1288, 1247, 1150, 839, 809, 789, 753 cm⁻; ¹H NMR (400 MHz, CDCl₃) δ ppm 7.54-7.43 (m, 3H), 7.20 (d, J=3.2 Hz, 1H), 7.15 (d, J=8.6 Hz, 1H), 6.91 (d, J=8.7 Hz, 2H), 6.57 (d, J=2.7 Hz, 1H), 3.83 (s, 3H), 3.78-3.66 (m, 1H), 3.33-3.17 (m, 1H), 3.05-2.93 (m, 1H), 2.78-2.66 (m, 1H), 1.03 (t, J=7.1 Hz, 3H), 0.94 (s, 9H), 0.65 (t, J=7.1 Hz, 3H), 0.64 (s, 3H), 0.58 (s, 3H); ¹³C NMR (101 MHz, CDCl₃) δ ppm 170.17, 158.56, 140.26, 133.58, 131.94, 130.29 (2C), 129.36, 129.30, 127.41, 122.98, 114.10, 113.53 (2C), 104.05, 55.27, 42.35, 38.19, 26.23 (3C), 19.43, 13.65, 12.37, −3.96, −4.02. MS EI m/z (rel. int.) 436 (M⁺, 38), 364 (100), 321 (16), 258 (17), 73 (31), 57 (16); HRMS m/z (ESI, [M+1]⁺) calcd for C₂₆H₃₇N₂O₂Si, 437.2624. found 437.2626.

### N-Ethyl-N-cumyl-2-phenylbenzamide

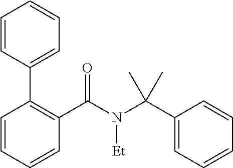 [SMILES: CCN(C(=O)c1ccccc1-c1ccccc1)C(C)(C)c1ccccc1]

Pale solid. mp 121-122° C. (EtOAc/hexanes); IR (KBr) νₘₐₓ 2980, 1638, 1395, 1287, 748, 699 cm⁻¹; NMR (400 MHz, CDCl₃) δ ppm 7.56-7.28 (m, 9H), 7.27-7.18 (m, 2H), 7.15 (t, J=6.7 Hz, 1H), 7.01 (d, J=6.5 Hz, 2H), 3.05 (m, 2H), 1.65 (s, 3H), 1.61 (s, 3H), 0.86 (t, J=6.6 Hz, 3H); ¹³C NMR (101 MHz, CDCl₃) δ ppm 170.99, 148.43, 140.16, 138.02, 137.94, 129.63, 129.36, 128.38, 128.36, 128.05, 127.41, 127.15, 127.09, 125.69, 124.32, 61.74, 41.30, 29.55, 26.91, 16.61. MS EI m/z (rel. int.) 343 (M⁺, 7), 238 (25), 224 (75), 181 (100), 153 (17), 152 (25), 119 (20); HRMS m/z (EI, M⁺) calcd for C₂₄H₂₅NO, 343.1936. found 343.1935.

### N,N-Diethyl-2-methoxy-5-phenylbenzamide

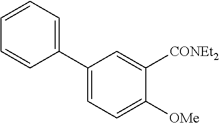

Light yellow solid. mp 85-87° C. (EtOAc/hexanes); IR (KBr) νₘₐₓ 2973, 2935, 1633, 1485, 1475, 1461, 1436, 1275, 1251, 1087, 1020, 763, 699 cm⁻; ¹H NMR (400 MHz, CDCl₃) δ ppm 7.60-7.49 (m, 3H), 7.48-7.36 (m, 3H), 7.31 (t, J=7.3 Hz, 1H), 6.97 (d, J=8.6 Hz, 1H), 3.85 (s, 3H), 3.66-3.52 (m, 2H), 3.25-3.11 (m, 2H), 1.26 (t, J=7.1 Hz, 3H), 1.05 (t, J=7.1 Hz, 3H); ¹³C NMR (101 MHz, CDCl₃) δ ppm 168.53, 154.67, 140.11, 133.82, 128.70 (2C), 128.34, 127.25, 126.87, 126.66 (2C), 126.07, 111.27, 55.65, 42.79, 38.81, 13.98, 12.88. MS EI m/z (rel. int.) 283 (M⁺, 24), 282 (23), 211 (100); HRMS m/z (EI, M⁺) calcd for C₁₈H₂₁NO₂, 283.1572. found 283.1575.

### N,N-Diethyl-2-methoxy-5-(4-methoxyphenyl)benzamide

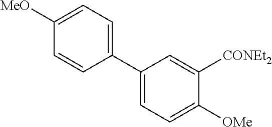

Colorless solid. mp 58-60° C. (EtOAc/hexanes); IR (KBr) νₘₐₓ 2971, 2936, 1633, 1609, 1494, 1474, 1462, 1438, 1276, 1244, 1181, 1087, 1051, 1021, 822 cm⁻¹; ¹H NMR (400 MHz, CDCl₃) δ ppm 7.54-7.41 (in, 3H), 7.38 (d, J=2.2 Hz, 1H), 7.01-6.85 (m, 3H), 3.85 (s, 3H), 3.84 (s, 3H), 3.64-3.50 (m, 2H), 3.24-3.11 (m, 2H), 1.26 (t, J=7.1 Hz, 3H), 1.05 (t, J=7.1 Hz, 3H); ¹³C NMR (101 MHz, CDCl₃) δ ppm 168.62, 158.82, 154.21, 133.54, 132.74, 127.88, 127.69 (2C), 127.19, 125.66, 114.15 (2C), 111.27, 55.65, 55.29, 42.79, 38.79, 13.98, 12.88. MS EI m/z (rel. int.) 313 (M⁺, 32), 312 (25), 241 (100), 183 (15), 139 (26); HRMS m/z (ESI, [M+1]⁺) calcd for C₁₉H₂₄NO₃, 314.1756. found 314.1746.

### N,N-Diethyl-2-phenyl-5-(4-methoxyphenyl)benzamide

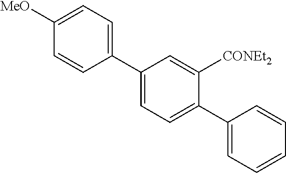

Light yellow solid. mp 117-118° C. (EtOAc/hexanes); IR (KBr) νₘₐₓ 2972, 2933, 1627, 1521, 1473, 1460, 1439, 1317, 1290, 1272, 1245, 1181, 1093, 826, 773, 702 cm⁻¹; ¹H NMR (400 MHz, CDCl₃) δ ppm 7.63 (dd, J=8.0, 1.9 Hz, 1H), 7.61-7.48 (m, 5H), 7.45 (d, J=8.0 Hz, 1H), 7.42-7.29 (m, 3H), 6.99 (d, J=8.7 Hz, 2H), 3.85 (s, 3H), 3.81-3.69 (m, 1H), 3.11-2.89 (m, 2H), 2.76-2.58 (m, 1H), 0.92 (t, J=7.1 Hz, 3H), 0.74 (t, J=7.1 Hz, 3H); ¹³C NMR (101 MHz, CDCl₃) δ ppm 170.54, 159.38, 139.97, 139.45, 136.67, 136.56, 132.40, 129.82, 128.78 (2C), 128.29 (2C), 128.02 (2C), 127.47, 127.05, 125.08, 114.27 (2C), 55.31, 42.26, 38.32, 13.39, 11.94. MS EI m/z (rel. int.) 359 (M⁺, 54), 358 (47), 287 (100), 216 (29), 215 (71), 72 (28), 56 (37); HRMS m/z (ESI, [M+1]⁺) calcd for C₂₄H₂₆NO₂, 360.1963. found 360.1955.

### N,N-Diethyl-2-phenyl-1-naphthamide

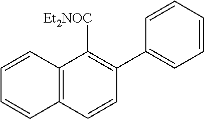

Light yellow oil. IR (KBr) νₘₐₓ 2974, 1625, 1480, 1429, 1285, 818, 749, 731 cm⁻¹; ¹H NMR (400 MHz, CDCl₃) δ ppm 7.96-7.81 (m, 3H), 7.62 (d, J=7.0 Hz, 2H), 7.57-7.46 (m, 3H), 7.42 (t, J=7.2 Hz, 2H), 7.37 (t, J=7.2 Hz, 1H), 3.90-3.71 (m, 1H), 3.29-3.11 (m, 1H), 3.02-2.86 (m, 1H), 2.75-2.57 (m, 1H), 0.98 (t, J=7.1 Hz, 3H), 0.62 (t, J=7.1 Hz, 3H); ¹³C NMR (101 MHz, CDCl₃) 6 ppm 169.23, 140.05, 135.29, 132.82, 132.57, 130.11, 129.26 (2C), 128.62, 128.19 (2C), 127.91, 127.46, 127.32, 126.99, 126.19, 125.56, 42.36, 38.22, 13.54, 12.07. MS EI m/z (rel. int.) 303 (M⁺, 27), 232 (12), 231 (100), 203 (13), 202 (32); HRMS m/z (EI, M⁺) calcd for C₂₁H₂₁NO, 303.1623. found 303.1624.

### N,N-Diethyl-1-phenyl-2-naphthamide

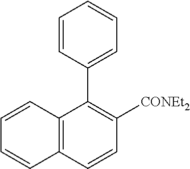

Light yellow solid. mp 121-122° C. (EtOAc/hexanes); IR (KBr) νₘₐₓ 2974, 2933, 1629, 1478, 1428, 1380, 1286, 1103, 818, 763, 705 cm⁻¹; NMR (400 MHz, CDCl₃) δ ppm 7.91 (d, J=8.3 Hz, 1H), 7.90 (d, J=7.6 Hz, 1H), 7.69 (d, J=8.4 Hz, 1H), 7.60-7.29 (m, 8H), 3.90-3.75 (m, 1H), 3.26-3.03 (m, 1H), 2.91-2.61 (m, 2H), 0.89 (t, J=7.1 Hz, 3H), 0.68 (t, J=7.1 Hz, 3H); ¹³C NMR (101 MHz, CDCl₃) δ ppm 170.20, 137.13, 135.47, 134.22, 133.38, 131.96, 131.20, 129.69, 128.61, 128.20, 128.02, 127.62, 127.28, 126.54, 126.47, 126.21, 123.35, 42.25, 37.76, 13.71, 11.70. MS EI m/z (rel. int.) 303 (M⁺, 28), 302 (26), 232 (15), 231 (100), 203 (12), 202 (38); HRMS m/z (EI, M⁺) calcd for C₂₁H₂₁NO, 303.1623. found 303.1635.

### N,N-Diethyl-3-phenyl-2-naphthamide

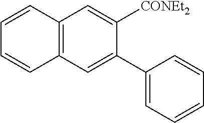

Light yellow oil. IR (KBr) νₘₐₓ 2974, 2932, 1626, 1478, 1442, 1423, 1286, 1086, 893, 775, 751, 700 cm⁻¹; ¹H NMR (400 MHz, CDCl₃) δ ppm 7.93-7.84 (m, 4H), 7.58 (dd, J=8.2, 1.5 Hz, 2H), 7.55-7.49 (m, 2H), 7.45-7.34 (m, 3H), 3.89-3.75 (m, 1H), 3.10-2.91 (m, 2H), 2.73-2.58 (m, 1H), 0.90 (t, J=7.1 Hz, 3H), 0.75 (t, J=7.1 Hz, 3H); ¹³C NMR (101 MHz, CDCl₃) δ ppm 170.38, 139.79, 136.48, 135.02, 133.27, 132.16, 129.09 (2C), 128.44, 128.29 (2C), 127.85 (2C), 127.53, 126.88, 126.52, 126.37, 42.27, 38.32, 13.34, 11.93. MS EI m/z (rel. int.) 303 (M⁺, 44), 302 (38), 232 (14), 231 (100), 203 (20), 202 (41); HRMS m/z (EI, M⁺) calcd for C₂₁H₂₁NO, 303.1623. found 303.1624.

### N,N-Dimethyl-1-phenyl-2-naphthamide

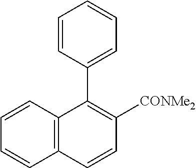

Light yellow solid. mp 86-87° C. (EtOAc/hexanes); IR (KBr) νₘₐₓ 3065, 2927, 1635, 1502, 1493, 1443, 1396, 1264, 1095, 822, 763, 702 cm⁻¹; ¹H NMR (400 MHz, CDCl₃) δ ppm 7.90 (d, J=8.3 Hz, 1H), 7.89 (d, J=7.9 Hz, 1H), 7.73 (d, J=8.5 Hz, 1H), 7.62-7.48 (m, 2H), 7.48-7.29 (m, 6H), 2.79 (s, 3H), 2.57 (s, 3H); ¹³C NMR (101 MHz, CDCl₃) δ ppm 171.08, 137.26, 135.84, 133.82, 133.59, 131.84, 130.78, 129.82, 128.67, 128.32, 128.06, 127.74, 127.40, 126.61, 126.51, 126.33, 123.63, 38.32, 34.24. MS EI m/z (rel. int.) 275 (M⁺, 32), 232 (18), 231 (100), 203 (18), 202 (80), 201 (22), 200 (21), 72 (19); HRMS m/z (EI, M⁺) calcd for C₁₉H₁₇NO, 275.1310. found 275.1310.

### N,N-Dimethyl-1-o-tolyl-2-naphthamide

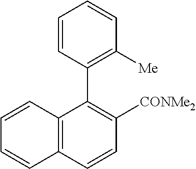

Light yellow solid. mp 94-07° C. (EtOAc/hexanes); IR (KBr) νₘₐₓ 3055, 2926, 1637, 1503, 1445, 1397, 1379, 1111, 1082, 822, 759, 730 cm⁻¹; ¹H NMR (400 MHz, CDCl₃) δ ppm 7.91 (d, J=8.4 Hz, 1H), 7.90 (d, J=8.1 Hz, 1H), 7.53-7.48 (in, 1H), 7.47-7.01 (m, 711), 2.83 (s, 3H), 2.81-2.60 (m, 3H), 2.04 (s, 3H) (atropisomers involved); ¹³C NMR (101 MHz, CDCl₃) δ ppm 170.79, 136.93 (brs), 135.79 (brs), 133.83 (brs), 133.15, 132.04 (brs), 129.97 (brs), 128.79 (brs), 128.07, 128.03, 127.99, 127.15 (brs), 126.68, 126.41, 126.32, 125.07 (brs), 123.46 (brs), 38.58 (brs), 34.29, 20.13 (atropisomers involved). MS EI m/z (rel. int.) 289 (M⁺, 14), 246 (20), 245 (100), 244 (46), 216 (25), 215 (94), 213 (25), 202 (56), 189 (19), 72 (32); HRMS m/z (EI, M⁺) calcd for C₂₀H₁₉NO, 289.1467. found 289.1455.

### N,N-Diethyl-1-(o-tolyl)-2-naphthamide

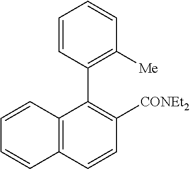

Pale solid. mp 150-152° C. (EtOAc/hexanes); IR (KBr) νₘₐₓ 2974, 2933, 1631, 1489, 1477, 1457, 1428, 1379, 1285, 1115, 1098, 818, 758, 729 cm⁻¹; ¹H NMR (400 MHz, CDCl₃) δ ppm 7.99-7.79 (m, 2H), 7.58-6.93 (m, 8H), 3.94-3.57 (m, 1H), 3.46-2.63 (m, 31), 2.18-1.82 (m, 3H), 1.17-0.80 (m, 3H), 0.67 (t, J=7.0 Hz, 3H) (atropisomers involved); ¹³C NMR (101 MHz, CDCl₃) δ ppm 170.02, 138.74, 137.05, 136.08, 135.53, 134.00, 133.13, 132.19, 131.98, 130.01, 129.50, 128.72, 128.03, 127.90, 126.63, 126.48, 126.21, 125.71, 124.71, 123.57, 122.94, 42.55, 42.10, 37.65, 20.22, 20.07, 13.87, 11.70 (atropisomers involved). MS EI m/z (rel. int.) 317 (M⁺, 31), 316 (24), 245 (100), 244 (31), 215 (27), 202 (26); HRMS m/z (EI, M⁺) calcd for C₂₂H₂₃NO, 317.1780. found 317.1790.

### N,N-Diethyl-1-(4-methylphenyl)-2-naphthamide

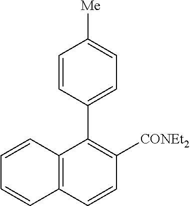

Light yellow solid. mp 181-183° C. (EtOAc/hexanes); IR (KBr) νₘₐₓ 2973, 2932, 1629, 1477, 1427, 1285, 1102, 817 cm⁻; ¹H NMR (400 MHz, CDCl₃) δ ppm 7.91 (d, J=8.2 Hz, 2H), 7.74 (d, J=8.3 Hz, 1H), 7.53 (t, J=7.2 Hz, 1H), 7.49-7.37 (m, 3H), 7.33-7.18 (m, 3H), 3.95-3.71 (m, 1H), 3.25-3.06 (m, 1H), 2.98-2.82 (m, 1H), 2.81-2.65 (m, 1H), 2.44 (s, 3H), 0.91 (t, J=7.0 Hz, 3H), 0.74 (t, J=7.0 Hz, 3H); ¹³C NMR (101 MHz, CDCl₃) δ ppm 170.34, 137.28, 135.59, 134.24, 134.11, 133.40, 132.10, 131.03, 129.55, 129.22, 127.98 (3C), 126.55, 126.43, 126.14, 123.40, 42.26, 37.78, 21.24, 13.72, 11.71. MS EI m/z (rel. int.) 317 (M⁺, 38), 316 (31), 246 (20), 245 (100), 215 (14), 202 (36); HRMS m/z (EI, calcd for C₂₂H₂₃NO, 317.1780. found 317.1786.

### 1-(3-(t-Butoxymethyl)phenyl)-N,N-diethyl-2-naphthamide

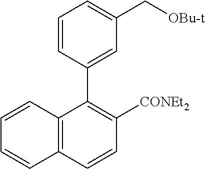

Light yellow solid. mp 1172-119° C. (EtOAc/hexanes); IR (KBr) νₘₐₓ 2973, 2933, 1631, 1477, 1428, 1378, 1363, 1285, 1195, 1103, 1070, 819, 756 cm⁻; ¹H NMR (400 MHz, CDCl₃) δ ppm 7.96-7.84 (m, 2H), 7.71 (t, J=7.9 Hz, 1H), 7.56-7.15 (m, 7H), 4.59-4.40 (m, 2H), 3.88-3.69 (m, 1H), 3.26-3.07 (m, 1H), 2.94-2.60 (m, 2H), 1.28 (d, 9H), 0.89 (m, 3H), 0.70 (t, J=7.0 Hz, 3H) (atropisomers involved); ¹³C NMR (101 MHz, CDCl₃) δ ppm 170.24, 140.32, 139.25, 137.01, 136.80, 135.72, 135.54, 134.19, 134.05, 133.39, 133.32, 132.04, 131.93, 130.06, 129.83, 128.51, 128.41, 128.38, 128.13, 128.07, 127.93, 127.37, 126.68, 126.60, 126.46, 126.43, 126.18, 123.44, 123.21, 73.35, 63.96, 63.90, 42.42, 42.38, 37.96, 37.76, 27.64, 13.73, 13.71, 11.76, 11.71 (atropisomers involved). MS EI m/z (rel. int.) 389 (M⁺, 25), 315 (14), 244 (31), 243 (100); HRMS m/z (EI, M⁺) calcd for C₂₆H₃₁NO₂, 389.2355. found 389.2368.

### N,N-Diethyl-1-(4-trifluoromethylphenyl)-2-naphthamide

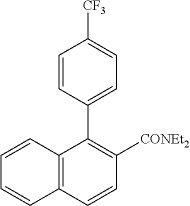

Pale solid mp 111-112° C. (EtOAc/hexanes); IR (KBr) νₘₐₓ 2977, 2935, 1630, 1478, 1430, 1326, 1166, 1126, 1106, 1067, 818 cm⁻¹; ¹H NMR (400 MHz, CDCl₃) δ ppm 7.95 (d, J=8.5 Hz, 1H), 7.92 (d, J=8.4 Hz, 1H), 7.79-7.64 (m, 3H), 7.59 (d, J=8.3 Hz, 1H), 7.54 (d, J=7.3 Hz, 1H), 7.50-7.39 (m, 3H), 3.91-3.69 (m, 1H), 3.21-3.02 (m, 1H), 2.93-2.63 (m, 2H), 0.92 (t, J=7.0 Hz, 3H), 0.67 (t, J=7.0 Hz, 3H); ¹³C NMR (101 MHz, CDCl₃) δ ppm 169.72, 141.09, 134.37, 133.95, 133.33, 131.77 (brs), 131.56, 130.03 (brs), 129.96 (q, ²J_{C-F}=32.5 Hz), 128.88, 128.23, 126.99, 126.49, 125.95, 125.56 (brs), 124.27 (brs), 124.15 (q, ¹J_{C-F}=272.2 Hz), 123.17, 42.33, 37.86, 13.75, 11.55. MS EI m/z (rel. int.) 371 (M⁺, 61), 370 (71), 300 (21), 299 (100), 251 (21), 202 (47); HRMS m/z (EI, M⁺) calcd for C₂₂H₂₀F₃NO, 371.1497. found 371.1513.

### N,N-Diethyl-1-(4-(dimethylamino)phenyl)-2-naphthamide

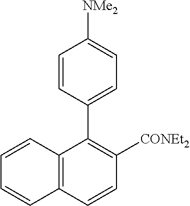

Pale solid. mp 140-141° C. (EtOAc/hexanes); IR (KBr) νₘₐₓ 2972, 2932, 1627, 1612, 1523, 1477, 1428, 1380, 1349, 1282, 817 cm⁻¹; ¹H NMR (400 MHz, CDCl₃) δ ppm 7.92-7.77 (m, 3H), 7.49 (t, J=7.3 Hz, 1H), 7.46-7.35 (m, 3H), 7.20 (d, J=8.1 Hz, 1H), 6.80 (t, J=9.5 Hz, 2H), 3.92-3.71 (m, 1H), 3.21-3.06 (m, 1H), 2.99 (s, 6H), 2.94-2.84 (m, 1H), 2.77-2.64 (m, 1H), 0.86 (t, J=6.9 Hz, 3H), 0.78 (t, J=6.9 Hz, 31-1); ¹³C NMR (101 MHz, CDCl₃) δ ppm 170.76, 150.05, 135.93, 134.28, 133.52, 132.41, 131.83, 130.54, 127.93, 127.50, 126.79, 126.22, 126.02, 125.03, 123.60, 112.53, 111.44, 42.20, 40.61, 37.84, 13.71, 12.03. MS EI m/z (rel. int.) 346 (M⁺, 84), 275 (18), 274 (100), 202 (14); HRMS m/z (EI, M⁺) calcd for C₂₃H₂₆N₂O, 346.2045. found 346.2049.

### N,N-Diethyl-1-(3-methoxyphenyl)-2-naphthamide

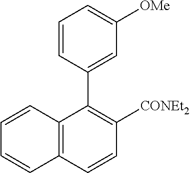

Colorless solid. mp 91-93° C. (EtOAc/hexanes); IR (KBr) νₘₐₓ 2972, 2934, 1629, 1578, 1478, 1463, 1429, 1378, 1285, 1251, 1047, 819 cm⁻¹; ¹H NMR (400 MHz, CDCl₃) δ ppm 7.98-7.81 (m, 2H), 7.75 (t, J=7.1 Hz, 1H), 7.51 (t, J=7.3 Hz, 1H), 7.47-7.28 (m, 3H), 7.19-7.08 (m, 1H), 7.01-6.83 (m, 2H), 3.93-3.73 (m, 4H), 3.25-3.05 (m, 1H), 2.91-2.63 (m, 2H), 0.96-0.82 (m, 3H), 0.73 (t, J=6.9 Hz, 3H) (atropisomers involved); ¹³C NMR (101 MHz, CDCl₃) δ ppm 170.22, 170.16, 159.55, 158.68, 138.41, 135.41, 135.24, 134.11, 133.36, 131.82, 129.62, 128.30, 128.23, 127.99, 126.55, 126.52, 126.48, 126.22, 123.80, 123.37, 123.30, 122.12, 115.75, 115.67, 114.30, 112.76, 55.28, 42.41, 42.37, 37.89, 37.79, 13.76, 11.72 (atropisomers involved). MS EI m/z (rel. int.) 333 (M⁺, 39), 332 (39), 262 (26), 261 (100), 246 (18), 218 (22), 189 (23); HRMS m/z (EI, M⁺) calcd for C₂₂H₂₃NO₂, 333.1729. found 333.1726.

### N,N-Diethyl-1-(4-methoxyphenyl)-2-naphthamide

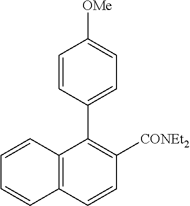

Pale solid. mp 114-116° C. (EtOAc/hexanes); IR (KBr) νₘₐₓ 2972, 2933, 1628, 1514, 1477, 1429, 1380, 1286, 1246, 1178, 1102, 1034, 819, 757 cm⁻¹; ¹H NMR (400 MHz, CDCl₃) δ ppm 7.88 (d, J=7.9 Hz, 2H), 7.72 (d, J=8.1 Hz, 1H), 7.57-7.35 (m, 4H), 7.25 (d, J=7.8 Hz, 1H), 6.98 (t, J=8.4 Hz, 2H), 3.86 (s, 3H), 3.84-3.73 (m, 1H), 3.21-3.03 (m, 1H), 2.95-2.80 (m, 1H), 2.78-2.65 (m, 1H), 0.88 (t, J=6.5 Hz, 3H), 0.75 (t, J=6.5 Hz, 3H); ¹³C NMR (101 MHz, CDCl₃) δ ppm 170.39, 159.15, 135.20, 134.39, 133.42, 132.35, 132.23, 130.82, 129.38, 128.01, 127.96, 126.48, 126.46, 126.14, 123.40, 113.41, 113.39, 55.31, 42.24, 37.82, 13.73, 11.92. MS EI m/z (rel. int.) 333 (M⁺, 32), 332 (27), 262 (23), 261 (100), 218 (23), 189 (25); HRMS m/z (EI, M⁺) calcd for C₂₂H₂₃NO₂, 333.1729. found 333.1721.

### N,N-Diethyl-1-(2-fluorophenyl)-2-naphthamide

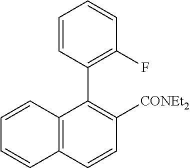

Pale solid. mp 137-140° C. (EtOAc/hexanes); IR (KBr) νₘₐₓ 2973, 2934, 1630, 1492, 1478, 1449, 1429, 1286, 1233, 1094, 819, 758, 731 cm⁻; NMR (400 MHz, CDCl₃) δ ppm 8.01-7.83 (m, 2H), 7.59-7.36 (m, 6H), 7.32-7.09 (m, 2H), 3.94-3.72 (m, 1H), 3.37-3.10 (m, 1H), 3.04-2.68 (m, 2H), 1.08-0.80 (m, 3H), 0.68 (t, J=6.5 Hz, 3H); ¹³C NMR (101 MHz, CDCl₃) 6 ppm 169.77, 159.98 (d, ¹J_{C-F}=245.8 Hz), 135.15, 133.54, 132.95, 131.81, 129.96 (d, ³J_{C-F}=8.0 Hz), 129.73, 128.99, 128.14, 126.89, 126.40, 126.00, 124.52 (d, ²J_{C-F}=17.4 Hz), 124.27, 123.23, 114.89 (d, ²J_{C-F}=21.9 Hz), 41.95, 37.77, 13.77, 11.74. MS EI m/z (rel. int.) 321 (M⁺, 31), 320 (34), 249 (100), 221 (14), 220 (35); HRMS m/z (EI, M⁺) calcd for C₂₁H₂₀FNO, 321.1529. found 321.1528.

### N,N-Diethyl-1-(4-fluorophenyl)-2-naphthamide

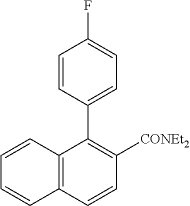

Pale solid. mp 103-104° C. (EtOAc/hexanes); IR (KBr) νₘₐₓ 2975, 2934, 1628, 1512, 1478, 1429, 1381, 1287, 1222, 1159, 1103, 819, 756, 728 cm⁻¹; ¹H NMR (400 MHz, CDCl₃) δ ppm 7.98-7.83 (m, 2H), 7.64 (d, J=8.3 Hz, 1H), 7.60-7.49 (m, 2H), 7.48-7.40 (m, 2H), 7.36-7.27 (m, 1H), 7.22-7.07 (m, 2H), 3.90-3.71 (m, 1H), 3.20-3.03 (m, 1H), 2.94-2.64 (m, 2H), 0.91 (t, J=7.1 Hz, 3H), 0.75 (t, J=7.1 Hz, 3H); ¹³C NMR (101 MHz, CDCl₃) δ ppm 170.06, 162.43 (d, ¹J_{C-F}=246.9 Hz), 134.47, 134.35, 133.37, 133.05 (d, ³J_{C-F}=8.2 Hz), 133.04 (d, ⁴J_{C-F}=3.6 Hz) 132.01, 131.25 (d, ³J_{C-F}=7.9 Hz), 128.42, 128.13, 126.73, 126.31, 126.17, 123.25, 115.50 (d, ²J_{C-F}=21.2 Hz), 114.42 (d, ²J_{C-F}=21.6 Hz), 42.30, 37.88, 13.75, 11.84. MS EI m/z (rel. int.) 321 (M⁺, 38), 320 (36), 249 (100), 220 (36); HRMS m/z (EI, M⁺) calcd for C₂₁H₂₀FNO, 321.1529. found 321.1533.

### 1-(3,5-Difluorophenyl)-N,N-diethyl-2-naphthamide

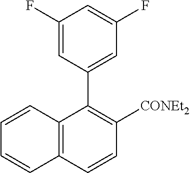

Light yellow oil. IR (KBr) νₘₐₓ 2975, 1624, 1588, 1481, 1432, 1386, 1285, 1119, 987, 819, 755 cm⁻; ¹H NMR (400 MHz, CDCl₃) δ ppm 7.94 (d, J=8.5 Hz, 1H), 7.91 (d, J=8.3 Hz, 1H), 7.65 (d, J=8.3 Hz, 1H), 7.54 (t, J=7.1 Hz, 1H), 7.51-7.41 (m, 2H), 7.13 (m, 1H), 6.92-6.83 (m, 2H), 3.96-3.80 (m, 1H), 3.25-3.03 (m, 1H), 2.97-2.74 (m, 2H), 0.96 (t, J=7.0 Hz, 3H), 0.83 (t, J=7.0 Hz, 3H); ¹³C NMR (101 MHz, CDCl₃) δ ppm 169.56, 162.94 (d, ¹J_{C-F}=246.2 Hz), 162.13 (d, ¹J_{C-F}=248.6 Hz), 140.49 (t, ³J_{C-F}=9.6 Hz, 1C), 134.25, 133.31, 133.02, 131.36, 129.04, 128.24, 127.10, 126.55, 125.80, 123.11, 114.60 (d, ²H_{C-F}=22.7 Hz, 1C), 112.62 (d, ²J_{C-F}=22.7 Hz, 1C), 103.17 (t, ²J_{C-F}=25.1 Hz), 42.49, 38.00, 13.80, 11.75. MS EI m/z (rel. int.) 339 (M⁺, 32), 338 (30), 267 (100), 238 (31); HRMS m/z (EI, M⁺) calcd for C₂₁H₁₉F₂NO, 339.1435. found 339.1420.

### N,N-Diethyl-1-(2-naphthyl)-2-naphthamide

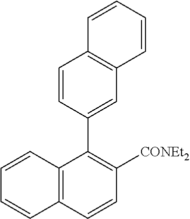

Pale solid. mp 159-161° C. (EtOAc/hexanes); IR (KBr) νₘₐₓ 2974, 1625, 1480, 1429, 1285, 1119, 1099, 921, 909, 819, 749, 731 cm⁻¹; ¹H NMR (400 MHz, CDCl₃) δ ppm 8.12-7.86 (m, 5H), 7.85-7.36 (m, 8H), 3.82-3.64 (m, 1H), 3.38-3.06 (m, 1H), 2.90-2.55 (m, 2H), 0.99-0.78 (m, 3H), 0.64-0.32 (m, 3H) (atropisomers involved); ¹³C NMR (101 MHz, CDCl₃) δ ppm 170.21, 135.54, 135.19, 135.06, 134.56, 134.52, 134.35, 133.44, 132.77, 132.61, 132.56, 132.19, 131.98, 130.30, 129.19, 128.51, 128.48, 128.39, 128.24, 128.08, 127.95, 127.87, 127.71, 127.42, 126.91, 126.63, 126.58, 126.27, 126.22, 126.13, 126.09, 123.59, 123.31, 42.46, 42.38, 37.90, 37.80, 13.77, 11.71, 11.50 (atropisomers involved). MS EI m/z (rel. int.) 353 (M⁺, 28), 282 (46), 281 (100), 252 (41), 126 (14); HRMS m/z (EI, M⁺) calcd for C₂₅H₂₃NO, 353.1780. found 353.1776.

### N,N-Diethyl-1-(furan-2-yl)-2-naphthamide

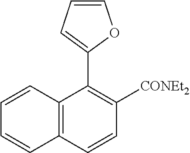

Light yellow oil. IR (KBr) νₘₐₓ 2974, 2934, 1629, 1479, 1429, 1287, 820, 739 cm⁻; ¹H NMR (400 MHz, CDCl₃) δ ppm 8.08-7.98 (m, 1H), 7.91 (d, J=8.4 Hz, 1H), 7.89-7.81 (m, 1H), 7.60 (brs, 1H), 7.57-7.47 (m, 2H), 7.42 (d, J=8.4 Hz, 1H), 6.70 (d, J=3.0 Hz, 1H), 6.55 (brs, 1H), 3.90-3.71 (m, 1H), 3.21-3.00 (m, 2H), 2.97-2.78 (m, 1H), 1.08 (t, J=7.0 Hz, 3H), 0.84 (t, J=7.0 Hz, 3H); ¹³C NMR (101 MHz, CDCl₃) δ ppm 170.26, 149.91, 142.61, 135.39, 133.34, 131.74, 129.45, 128.12, 127.08, 126.48, 126.23, 124.90, 123.58, 111.91, 111.20, 42.44, 38.40, 13.45, 12.30. MS EI m/z (rel. int.) 293 (M⁺, 31), 220 (98), 193 (59), 164 (78), 138 (15), 100 (19); HRMS m/z (EI, M⁺) calcd for C₁₉H₁₉NO₂, 293.1416. found 293.1429.

### N,N-Diethyl-1-(thiophen-3-yl)-2-naphthamide

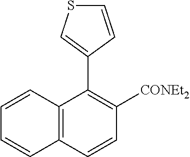

Light yellow solid. mp 71-73° C. (EtOAc/hexanes); IR (KBr) νₘₐₓ 2973, 2932, 1626, 1478, 1429, 1285, 1101, 818, 755, 653 cm⁻; ¹H NMR (400 MHz, CDCl₃) δ ppm 7.92 (d, J=8.4 Hz, 2H), 7.85 (d, J=8.3 Hz, 1H), 7.61-7.36 (m, 5H), 7.26 (brs, 1H), 3.97-3.78 (m, 1H), 3.19-3.03 (m, 1H), 3.02-2.87 (m, 1H), 2.85-2.69 (m, 1H), 0.96-0.80 (m, 6H); ¹³C NMR (101 MHz, CDCl₃) δ ppm 170.37, 136.99, 134.74, 133.32, 132.18, 130.58, 129.78 (brs), 128.34, 128.07, 126.68, 126.30, 126.29, 125.35 (brs), 124.88, 123.39, 42.29, 38.04, 13.70, 12.02; MS EI m/z (rel. int.) 309 (M⁺, 17), 238 (37), 237 (100), 208 (64), 165 (32), 57 (35), 56 (40); HRMS m/z (ESI, [M+1]⁺) calcd for C₁₉H₂₀NOS, 310.1265. found 310.1248.

### (E)-N,N-Dimethyl-1-styryl-2-naphthamide

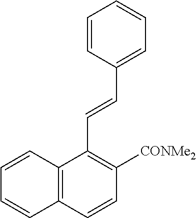

Pale solid. mp 114-116° C. (EtOAc/hexanes); IR (KBr) νₘₐₓ 3056, 2925, 1628, 1496, 1448, 1396, 1256, 1108, 1059, 975, 820, 751, 732, 697 cm⁻¹; ¹H NMR (400 MHz, CDCl₃) δ ppm 8.26-8.17 (m, 1H), 7.92-7.85 (m, 1H), 7.84 (d, J=8.4 Hz, 1H), 7.61 (d, J=16.4 Hz, 1H), 7.58-7.50 (m, 4H), 7.41 (d, J=8.4 Hz, 1H), 7.40 (t, J=8.1 Hz, 2H), 7.32 (t, J=7.3 Hz, 1H), 7.02 (d, J=16.4 Hz, 1H), 3.07 (s, 3H), 2.78 (s, 3H); ¹³C NMR (101 MHz, CDCl₃) 6 ppm 171.69, 137.20, 136.01, 133.50, 132.94, 131.61, 131.42, 128.74 (2C), 128.44, 128.19, 128.10, 126.73, 126.63 (2C), 126.47, 125.23, 124.09, 123.33, 38.16, 34.75. MS EI m/z (rel. int.) 301 (M⁺, 55), 257 (65), 256 (65), 229 (39), 228 (100), 227 (36), 226 (49), 105 (36), 77 (71), 72 (65); HRMS m/z (EI, M⁺) calcd for C₂₁H₁₉NO, 301.1467. found 301.1452.

### (E)-N,N-Diethyl-1-styryl-2-naphthamide

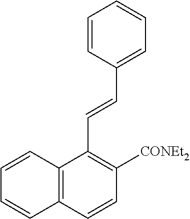

Pale solid. mp 86-89° C. (EtOAc/hexanes); IR (KBr) νₘₐₓ 2974, 2361, 2341, 1624, 1479, 1449, 1427, 1379, 1286, 1115, 975, 816, 750, 695 cm⁻¹; ¹H NMR (400 MHz, CDCl₃) δ ppm 8.25-8.15 (m, 1H), 7.92-7.85 (m, 1H), 7.83 (d, J=8.4 Hz, 1H), 7.64-7.46 (m, 5H), 7.44-7.34 (m, 3H), 7.30 (t, J=7.3 Hz, 1H), 7.05 (d, J=16.4 Hz, 1H), 4.00-3.79 (m, 1H), 3.30-3.10 (m, 2H), 3.09-2.95 (m, 1H), 1.10 (t, J=7.1 Hz, 3H), 0.98 (t, J=7.1 Hz, 3H); ¹³C NMR (101 MHz, CDCl₃) δ ppm 170.85, 137.06, 136.15, 133.44, 133.35, 131.49, 131.28, 128.67 (2C), 128.41, 128.03, 128.00, 126.68, 126.59 (2C), 126.35, 125.26, 124.00, 123.36, 42.61, 38.73, 13.86, 12.64. MS EI m/z (rel. int.) 329 (M⁺, 40), 258 (27), 257 (53), 256 (39), 229 (62), 228 (100), 227 (51), 226 (59), 105 (40), 78 (31), 77 (43), 57 (47), 56 (70); HRMS m/z (EI, M⁺) calcd for C₂₃H₂₃NO, 329.1780. found 329.1770.

### N,N-Dimethyl-2-phenyl-1-naphthamide

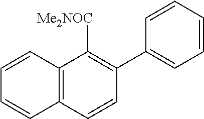

Light yellow solid. mp 133-134° C. (EtOAc/hexanes); IR (KBr) νₘₐₓ 1634, 1495, 1398, 765, 704 cm⁻¹; ¹H NMR (400 MHz, CDCl₃) δ ppm 7.97-7.83 (m, 3H), 7.59 (d, J=7.1 Hz, 2H), 7.57-7.48 (m, 3H), 7.44 (t, J=7.3 Hz, 2H), 7.37 (t, J=7.2 Hz, 1H), 2.98 (s, 3H), 2.43 (s, 3H); ¹³C NMR (101 MHz, CDCl₃) δ ppm 170.14, 140.14, 135.70, 132.60, 132.35, 129.91, 128.93, 128.86 (2C), 128.32 (2C), 127.95, 127.56, 127.28, 127.17, 126.29, 125.50, 37.68, 34.43. MS EI m/z (rel. int.) 275 (M⁺, 22), 231 (100), 203 (13), 202 (31); HRMS m/z (EI, calcd for C₁₉H₁₇NO, 275.1310. found 275.1309.

### 2-(2-Methylphenyl)-N,N-dimethyl-1-naphthamide

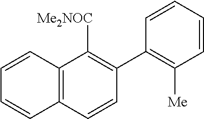

Light yellow solid. mp 108-109° C. (EtOAc/hexanes); IR (KBr) νₘₐₓ 2926, 1637, 1508, 1494, 1448, 1400, 1264, 1193, 1124, 830, 762, 729 cm⁻¹; ¹H NMR (400 MHz, CDCl₃) δ ppm 8.08-6.93 (m, 1011), 3.02-2.84 (m, 3H), 2.82-2.46 (m, 3H), 2.35-2.12 (in, 3H) (atropisomers involved); ¹³C NMR (101 MHz, CDCl₃) δ ppm 169.73, 169.60, 140.06, 138.77, 137.39, 136.40, 135.19, 134.81, 133.39, 133.29, 132.50, 132.39, 130.73, 130.20, 129.42, 128.20, 128.15, 128.00, 127.80, 127.69, 127.19, 127.05, 126.30, 126.21, 125.66, 125.56, 125.01, 124.69, 38.27, 37.72, 34.36, 34.12, 20.35, 20.25 (atropisomers involved). MS EI m/z (rel. int.) 289 (M⁺, 5), 245 (91), 244 (64), 216 (34), 215 (100), 202 (65), 72 (35); HRMS m/z (EI, M⁺) calcd for C₂₀H₁₉NO, 289.1467. found 289.1463.

### 2-(4-Methylphenyl)-N,N-dimethyl-1-naphthamide

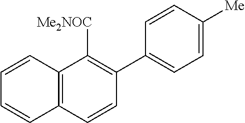

Light yellow solid. mp 145-146 (EtOAc/hexanes); IR (KBr) 2923, 1634, 1506, 1448, 1399, 1264, 1194, 1124, 812, 748, 730 cm⁻¹; ¹H NMR (400 MHz, CDCl₃) δ ppm 7.90 (d, J=8.6 Hz, 1H), 7.88-7.83 (m, 2H), 7.58-7.44 (m, 5H), 7.24 (d, J=7.9 Hz, 1H), 3.00 (s, 3H), 2.44 (s, 3H), 2.41 (s, 3H); ¹³C NMR (101 MHz, CDCl₃) δ ppm 170.32, 137.32, 137.26, 135.75, 132.52, 132.18, 129.99, 129.09 (2C), 128.87, 128.74 (2C), 127.95, 127.42, 127.11, 126.16, 125.47, 37.71, 34.49, 21.18. MS EI m/z (rd. int.) 289 (M⁺, 28), 245 (100), 215 (49), 202 (91); HRMS m/z (EI, M⁺) calcd for C₂₀H₁₉NO, 289.1467. found 289.1465.

### 2-(4-(Trifluoromethyl)phenyl)-N,N-dimethyl-1-naphthamide

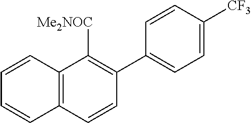

Light yellow solid. mp 184-186° C. (EtOAc/hexanes); IR (KBr) νₘₐₓ 2931, 1635, 1619, 1507, 1402, 1325, 1166, 1125, 1082, 1062, 1019, 820, 754, 733 cm⁻; ¹H NMR (400 MHz, CDCl₃) δ ppm 7.94 (d, J=8.5 Hz, 1H), 7.92-7.82 (m, 2H), 7.75-7.65 (m, 4H), 7.61-7.52 (m, 2H), 7.50 (d, J=8.5 Hz, 1H), 3.00 (s, 3H), 2.46 (s, 3H); ¹³C NMR (101 MHz, CDCl₃) δ ppm 169.74, 143.83, 134.15, 132.91, 132.89, 129.77, 129.72 (q, ²J_{C-F}=32.49 Hz), 129.27 (2C), 129.22, 128.08, 127.54, 126.83, 126.82, 125.52, 125.29 (q, ³J_{C-F}=3.69 Hz, 2C), 121.46 (q, ¹J_{C-F}=272.03 Hz), 37.73, 34.49. MS EI m/z (rel. int.) 343 (M⁺, 31), 299 (100), 251 (37), 202 (67), 69 (42); HRMS m/z (EI, M⁺) calcd for C₂₀H₁₆F₃NO, 343.1184. found 343.1172.

### 2-(4-(Dimethylamino)phenyl)-N,N-dimethyl-1-naphthamide

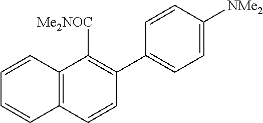

Light yellow solid. mp 156-157° C. (EtOAc/hexanes); IR (KBr) νₘₐₓ 2923, 2890, 1633, 1610, 1527, 1506, 1445, 1398, 1360, 1199, 1125, 815, 752 cm⁻¹; ¹H NMR (400 MHz, CDCl₃) δ ppm 7.92-7.80 (m, 3H), 7.57-7.42 (m, 5H), 6.79 (d, J=8.8 Hz, 2H), 3.03 (s, 3H), 3.01 (s, 6H), 2.43 (s, 3H); ¹³C NMR (101 MHz, CDCl₃) δ ppm 170.74, 149.83, 135.97, 132.15, 131.37, 130.18, 129.65 (2C), 128.75, 127.97, 127.85, 127.45, 126.91, 125.71, 125.31, 112.18 (2C), 40.37, 37.67, 34.51. MS EI m/z (rel. int.) 318 (M⁺, 68), 274 (100), 230 (25), 203 (28), 202 (87), 201 (22), 200 (20), 189 (23); HRMS m/z (EI, M⁺) calcd for C₂₁H₂₂N₂O, 318.1732. found 318.1737.

### 2-(3-Methoxyphenyl)-N,N-dimethyl-1-naphthamide

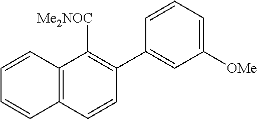

Light yellow oil. IR (KBr) νₘₐₓ 2933, 1633, 1611, 1596, 1581, 1509, 1490, 1465, 1399, 1290, 1261, 1222, 1046, 784, 702 cm⁻¹; ¹H NMR (400 MHz, CDCl₃) δ ppm 7.91 (d, J=8.5 Hz, 1H), 7.89-7.83 (m, 2H), 7.58-7.47 (m, 3H), 7.34 (t, J=8.1 Hz, 1H), 7.19-7.13 (m, 2H), 6.96-6.89 (m, 1H), 3.85 (s, 3H), 3.00 (s, 3H), 2.46 (s, 3H); ¹³C NMR (101 MHz, CDCl₃) δ ppm 170.17, 159.43, 141.57, 135.60, 132.66, 132.36, 129.90, 129.34, 128.91, 127.96, 127.21, 127.18, 126.32, 125.49, 121.27, 114.05, 113.66, 55.31, 37.76, 34.50. MS EI m/z (rel. int.) 305 (M⁺, 28), 261 (100), 218 (27), 202 (32), 189 (71), 72 (17); HRMS m/z M⁺) calcd for C₂₀H₁₉NO₂, 305.1416. found 305.1429.

### 2(4-Methoxyphenyl)-N,N-dimethyl-1-naphthamide

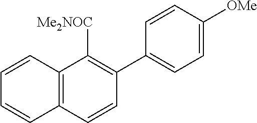

Light yellow solid. mp 194-195° C. (EtOAc/hexanes); IR (KBr) νₘₐₓ 2932, 1633, 1610, 1517, 1462, 1399, 1291, 1251, 1181, 1125, 1031, 821, 749, 730 cm⁻; ¹H NMR (400 MHz, CDCl₃) δ ppm 7.89 (d, J=8.8 Hz, 1H), 7.87-7.82 (m, 2H), 7.55-7.46 (m, 5H), 6.97 (d, J=8.8 Hz, 2H), 3.86 (s, 3H), 3.00 (s, 3H), 2.43 (s, 3H); ¹³C NMR (101 MHz, CDCl₃) δ ppm 170.39, 159.16, 135.39, 132.59, 132.42, 132.04, 130.07 (2C), 130.02, 128.87, 127.94, 127.36, 127.12, 126.09, 125.41, 113.81 (2C), 55.25, 37.68, 34.48. MS EI m/z (rel. int.) 305 (M⁺, 36), 262 (31), 261 (100), 218 (23), 202 (25), 190 (28), 189 (87), 72 (29); HRMS m/z (EI, M⁺) calcd for C₂₀H₁₉NO₂, 305.1416. found 305.1429.

### 2-(2-Fluorophenyl)-N,N-dimethyl-1-naphthamide

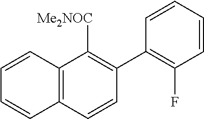

Light yellow solid. mp 105-106° C. (EtOAc/hexanes); IR (KBr) νₘₐₓ 2927, 1637, 1496, 1450, 1400, 1261, 1206, 1195, 806, 760 cm⁻¹; ¹H NMR (400 MHz, CDCl₃) δ ppm 7.93-7.87 (m, 2H), 7.87-7.80 (m, 1H), 7.60-7.46 (m, 4H), 7.41-7.31 (m, 1H), 7.24-7.12 (m, 2H), 2.96 (s, 3H), 2.57 (s, 3H); ¹³C NMR (101 MHz, CDCl₃) δ ppm 169.50, 159.57 (d, ¹J_{C-F}=246.3 Hz), 133.74, 132.87, 131.92 (d, ⁴J_{C-F}=3.0 Hz), 130.01, 129.88, 129.70 (d, ³J_{C-F}=8.1 Hz), 128.20, 128.08, 127.97 (d, ⁴H_{C-F}=2.3 Hz), 127.36 (d, ²J_{C-F}=14.9 Hz), 127.15, 126.61, 125.45, 124.00 (d, ³J_{C-F}=3.6 Hz), 115.49 (d, ²J_{C-F}=22.1 Hz), 37.76, 34.39. MS EI m/z (rel. int.) 293 (M⁺, 28), 249 (96), 221 (38), 220 (100), 219 (20), 218 (22); HRMS m/z (EI, M⁺) calcd for C₁₉H₁₆FNO, 293.1216. found 293.1230.

### 2-(4-Fluorophenyl)-N,N-dimethyl-1-naphthamide

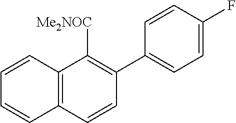

Light yellow solid. mp 103-104° C. (EtOAc/hexanes); IR (KBr) 3058, 2928, 1633, 1605, 1509, 1400, 1225, 1161, 823, 749, 731 cm⁻¹; ¹H NMR (400 MHz, CDCl₃) δ ppm 7.91 (d, J=83 Hz, 1H), 7.89-7.82 (m, 2H), 7.60-7.50 (m, 4H), 7.48 (d, J=8.5 Hz, 1H), 7.18-7.08 (m, 2H), 3.00 (s, 3H), 2.44 (s, 3H); ¹³C NMR (101 MHz, CDCl₃) δ ppm 170.06, 162.43 (d, ¹J_{C-F}=247.2 Hz), 136.19 (d, ⁴J_{C-F}=3.3 Hz), 134.61, 132.61, 132.48, 130.60 (d, ³J_{C-F}=8.1 Hz, 2C), 129.86, 129.02, 128.00, 127.33, 127.13, 126.43, 125.44, 115.42, 115.32 (d, ²J_{C-F}=21.4 Hz, 2C), 37.68, 34.46. MS EI m/z (rel. int.) 293 (M⁺, 22), 249 (79), 221 (35), 220 (100), 219 (19), 218 (24); HRMS m/z (EI, M⁺) calcd for C₁₉H₁₆FNO, 293.1216. found 293.1216.

### 2-(Naphthalen-2-yl)-N,N-dimethyl-1-naphthamide

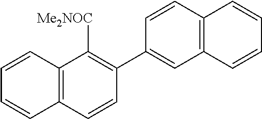

Light yellow solid. mp 168-169° C. (EtOAc/hexanes); IR (KBr) νₘₐₓ 3055, 2938, 1631, 1504, 1400, 1265, 1194, 1125, 909, 819, 744, 731 cm⁻¹; ¹H NMR (400 MHz, CDCl₃) δ ppm 8.07 (d, J=0.8 Hz, 1H), 7.96 (d, J=8.5 Hz, 1H), 7.94-7.85 (m, 5H), 7.75 (dd, J=8.5, 1.7 Hz, 1H), 7.64 (d, J=8.5 Hz, 1H), 7.61-7.49 (m, 4H), 2.94 (s, 3H), 2.43 (s, 3H); ¹³C NMR (101 MHz, CDCl₃) δ ppm 170.21, 137.66, 135.58, 133.29, 132.67, 132.66, 132.59, 130.03, 129.01, 128.32, 128.00, 127.96 (2C), 127.60, 127.54, 127.24, 126.94, 126.39, 126.25, 126.24, 125.56, 37.74, 34.49. MS EI m/z (rel. int.) 325 (M⁺, 34), 282 (28), 281 (100), 253 (28), 252 (77), 250 (32), 72 (20); HRMS m/z (EI, M⁺) calcd for C₂₃H₁₉NO, 325.1467. found 325.1468.

### N,N-Dimethyl-2-(thiophen-3-yl)-1-naphthamide

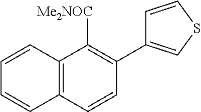

Light yellow oil. IR (KBr) νₘₐₓ 2926, 1630, 1508, 1399, 1263, 1194, 1125, 1017, 800, 784, 748, 641 cm⁻¹; ¹H NMR (400 MHz, CDCl₃) δ ppm 7.88 (d, J=8.5 Hz, 1H), 7.87-7.77 (m, 2H), 7.58 (d, J=8.5 Hz, 1H), 7.56-7.46 (m, 3H), 7.41-7.34 (m, 2H), 3.08 (s, 3H), 2.47 (s, 3H); ¹³C NMR (101 MHz, CDCl₃) δ ppm 170.50, 140.35, 132.54, 131.84, 130.30, 129.95, 128.87, 128.04, 127.97, 127.22, 126.72, 126.25, 125.65, 125.36, 123.45, 37.70, 34.59. MS EI m/z (rel. int.) 281 (M⁺, 35), 238 (29), 237 (100), 209 (30), 208 (90), 165 (53), 164 (31), 163 (40); HRMS m/z (EI, M⁺) calcd for C₁₇H₁₅NOS, 281.0874. found 281.0871.

### (E)-N,N-Dimethyl-2-styryl-1-naphthamide

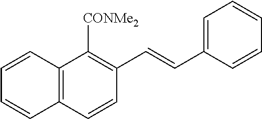

Light yellow oil. IR (KBr) νₘₐₓ 3057, 2928, 1633, 1510, 1496, 1449, 1399, 1263, 1190, 1123, 1023, 959, 909, 814, 742, 701 cm⁻¹; ¹H NMR (400 MHz, CDCl₃) δ ppm 7.87-7.79 (m, 3H), 7.71 (dd, J=75, 1.4 Hz, 1H), 7.57-7.44 (m, 4H), 7.38 (t, J=7.5 Hz, 2H), 7.30 (t, J=7.3 Hz, 1H), 7.27 (d, J=16.4 Hz, 1H), 7.21 (d, J=16.2 Hz, 1H), 3.33 (s, 3H), 2.74 (s, 3H); ¹³C NMR (101 MHz, CDCl₃)*δ ppm 170.22, 137.01, 132.95, 132.76, 131.54, 130.81, 129.75, 128.77, 128.68 (2C), 128.08, 128.03, 127.28, 126.76 (2C), 126.29, 125.13, 124.99, 12257, 38.02, 34.62. MS EI m/z (rel. int.) 301 (M⁺, 43), 257 (81), 256 (59), 229 (70), 228 (100), 227(1), 226 (78), 202 (35), 105 (70), 77(67), 72 (33), 51(38); HRMS m/z (EI, M⁺) calcd for C₂₁H₁₉NO, 301.1467. found 301.1478.

### N,N-Diethyl-3-methoxy-2-phenyl-1naphthamide

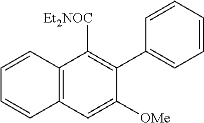

Pale solid. mp 106-107° C. (EtOAc/hexanes); IR (KBr) νₘₐₓ 2973, 2935, 1629, 1595, 1456, 1423, 1294, 1263, 1224, 1189, 1059, 762, 736, 700 cm⁻¹; ¹H NMR (400 MHz, CDCl₃) δ ppm 7.76 (t, J=9.0 Hz, 2H), 7.60-7.43 (m, 3H), 7.42-7.30 (m, 4H), 7.23 (s, 1H), 3.87 (s, 3H), 3.86-3.77 (m, 1H), 3.16-2.90 (m, 2H), 2.78-259 (m, 1H), 0.74 (t, J=7.1 Hz, 6H); ¹³C NMR (101 MHz, CDCl₃) ppm 168.33, 154.71, 135.69, 135.26, 134.23, 130.34, 128.49, 127.62 (2C), 127.44, 126.71 (3C), 125.50, 125.31, 124.50, 106.02, 55.70, 42.27, 37.70, 13.72, 11.76. MS EI m/z (rel. int.) 333 (M⁺, 50), 332 (21), 262 (26), 261 (100), 246 (34), 189 (19); HRMS m/z (EI, M⁺) calcd for C₂₂H₂₃NO₂, 333.1729. found 333.1732.

### N,N-Diethyl-4-methoxy-1-phenyl-2-naphthamide

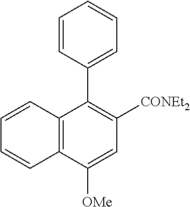

Colorless oil. IR (KBr) νₘₐₓ 2971, 2934, 1630, 1593, 1477, 1459, 1431, 1375, 1344, 1272, 1104, 772, 702 cm⁻¹; ¹H NMR (400 MHz, CDCl₃) ppm 8.33 (d, J=8.3 Hz, 1H), 7.65 (d, J=8.3 Hz, 1H), 758-7.47 (m, 2H), 7.47-7.28 (m, 5H), 6.81 (s, 1H), 4.05 (s, 3H), 3.91-3.78 (m, 1H), 3.27-3.11 (m, 1H), 2.87-2.62 (m, 2H), 0.90 (t, J=7.1 Hz, 3H), 0.67 (t, J=7.1 Hz, 3H); ¹³C NMR (101 MHz, CDCl₃) 6 ppm 170.30, 155.19, 137.20, 134.03, 132.93, 13148, 130.14, 128.58, 127.73, 127.32, 127.21, 126.97, 126.18, 125.51, 125.48, 121.90, 101.50, 55.64, 42.20, 37.69, 13.78, 11.65. MS EI m/z (rel. int.) 3333 (M⁺, 51), 318 (38), 262 (23), 261 (100), 246 (26), 189 (19); HRMS m/z (EI, M⁺) calcd for C₂₂H₂₃NO₂, 333.1729. found 333.1720.

### N,N-Diethyl-3-methoxy-1-phenyl-2-naphthamide

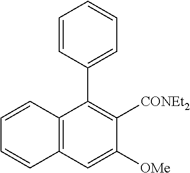

Light yellow oil. IR (KBr) νₘₐₓ 2976, 2535, 1633, 1597, 1478, 1461, 1419, 1295, 1233, 1161, 1087, 754, 702 cm⁻¹; ¹H NMR (400 MHz, CDCl₃) δ ppm 7.78 (d, J=8.1 Hz, 1H), 756 (d, J=7.5 Hz, 1H), 7.50 (d, J=8.5 Hz, 1H), 7.47-7.42 (m, 2H), 7.41-7.35 (m, 2H), 7.30-7.22 (m, 2H), 7.21 (s, 1H), 3.97 (s, 3H), 3.93-3.80 (m, 1H), 3.24-3.10 (m, 1H), 2.86-2.70 (m, 2H), 0.93 (t, J=7.1 Hz, 3H), 0.60 (t, J=7.1 Hz, 3H); ¹³C NMR (101 MHz, CDCl₃) 6 ppm 167.09, 153.35, 137.51, 136.71, 134.40, 131.10, 129.37, 128.46, 127.86, 127.59 (2C), 127.13, 126.78, 126.64, 126.50, 124.04, 105.39, 55.46, 42.20, 37.37, 13.27, 11.56. MS EI m/z (rel. int.) 333 (M⁺, 22), 302 (15), 262 (18), 261 (100), 256 (14), 189 (12); HRMS m/z (EI, M⁺) calcd for C₂₂H₂₃NO₂, 333.1729. found 333.1718.

### 4-Bromo-N,N-diethyl-1-methoxy-2-naphthamide

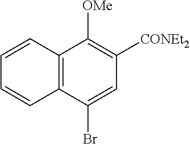

Yellow oil. IR (KBr) νₘₐₓ 2973, 2935, 1634, 1592, 1476, 1454, 1429, 1361, 1324, 1278, 1255, 1220, 1132, 1083, 763 cm⁻¹; ¹H NMR (400 MHz, CDCl₃) δ ppm 8.20 (d, J=9.1 Hz, 1H), 8.18 (d, J=9.1 Hz, 1H), 7.68-7.51 (m, 3H), 4.00 (s, 3H), 3.86-3.69 (m, 1H), 3.53-3.35 (m, 1H), 3.32-3.08 (m, 2H), 1.30 (t, J=7.1 Hz, 3H), 1.05 (t, J=7.1 Hz, 3H); ¹³C NMR (101 MHz, CDCl₃) 6 ppm 167.49, 151.45, 132.97, 128.95, 128.19, 128.15, 127.41, 127.11, 126.25, 122.87, 117.54, 62.77, 43.15, 39.18, 14.02, 12.74. MS EI m/z (rel. int.) 337 ([M+2]⁺, 14), 335 (M⁺, 17), 265 (89), 263 (87), 250 (24), 248 (25), 194 (26), 192 (30), 156 (23), 155 (24), 128 (30), 127 (23), 126 (65), 113 (62), 72 (31), 58 (34), 57 (100), 56 (100); HRMS m/z (ESI, [M+1]⁴) calcd for C₁₆H₁₉Br NO₂, 336.0599. found 336.0590.

### 6-Bromo-2-methoxy-N,N-diethyl-1-naphthamide

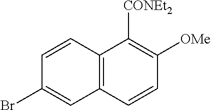

Pale solid. mp 109-110° C. (EtOAc/hexanes); IR (KBr) νₘₐₓ 2973, 2935, 1629, 1586, 1498, 1473, 1459, 1435, 1334, 1282, 1264, 1251, 1075, 887 cm⁻; ¹H NMR (400 MHz, CDCl₃) δ ppm 7.94 (s, 1H), 7.75 (d, J=9.1 Hz, 1H), 7.55-7.47 (m, 2H), 7.28 (d, J=9.1 Hz, 1H), 3.93 (s, 3H), 3.86-3.72 (m, 1H), 3.70-3.53 (m, 1H), 3.18-2.98 (m, 2H), 1.35 (t, J=7.1 Hz, 3H), 0.95 (t, J=7.1 Hz, 3H); ¹³C NMR (101 MHz, CDCl₃) 6 ppm 167.23, 152.65, 130.52, 129.90, 129.78, 129.48, 129.16, 125.59, 120.69, 117.62, 113.95, 56.32, 42.79, 38.90, 13.99, 13.00. MS EI m/z (rel. int.) 337 ([M+2]⁺, 22), 335 (M⁺, 25), 265 (96), 263 (100), 126 (52), 113 (40), 57 (62), 56 (68); HRMS m/z (EI, M⁺) calcd for C₁₆H₁₈BrNO₂, 335.0521. found 335.0525.

### 6-Bromo-2-methoxy-N,N-dimethyl-1-naphthamide

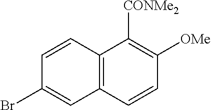

Pale solid. mp 124-125° C. (EtOAc/hexanes); IR (KBr) νₘₐₓ 2936, 1636, 1586, 1499, 1411, 1391, 1352, 1333, 1274, 1253, 1186, 1176, 1133, 1073, 1019, 903, 818 cm⁻¹; ¹H NMR (400 MHz, CDCl₃) δ ppm 7.94 (s, 1H), 7.76 (d, J=9.1 Hz, 1H), 7.56-7.45 (m, 2H), 7.29 (d, J=9.1 Hz, 1H), 3.94 (s, 3H), 3.24 (s, 3H), 2.78 (s, 3H); ¹³C NMR (101 MHz, CDCl₃) δ ppm 168.09, 152.81, 130.64, 129.94, 129.81, 129.42, 129.28, 125.67, 120.18, 117.69, 113.90, 56.44, 37.75, 34.61. MS EI m/z (rel. int.) 309 ([M+2]⁺, 22), 307 (M⁺, 28), 265 (100), 263 (100), 222 (17), 220 (15), 194 (19), 192 (15), 126 (65), 114 (24), 113 (52), 72 (51); HRMS m/z (EI, M⁺) calcd for C₁₄H₁₄BrNO₂, 307.0208. found 307.0202.

### N,N-Diethyl-1-methoxy-4-phenyl-2-naphthamide

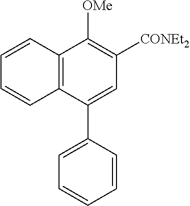

Colorless solid. mp 102-103° C. (EtOAc/hexanes); IR (KBr) νₘₐₓ 2973, 2935, 1631, 1476, 1457, 1429, 1369, 1271, 1221, 1082, 777, 755, 703 cm⁻¹; ¹H NMR (400 MHz, CDCl₃) δ ppm 8.26 (d, J=8.4 Hz, 1H), 7.90 (d, J=8.4 Hz, 1H), 7.56 (t, J=7.5 Hz, 1H), 7.52-7.37 (m, 6H), 7.29 (s, 1H), 4.07 (s, 3H), 3.89-3.70 (m, 1H), 3.57-3.41 (m, 1H), 3.40-3.15 (m, 2H), 1.32 (t, J=7.1 Hz, 3H), 1.08 (t, J=7.1 Hz, 3H); ¹³C NMR (101 MHz, CDCl₃) δ ppm 168.86, 151.11, 139.84, 136.61, 132.94, 130.02 (2C), 128.22 (2C), 127.97, 127.30, 126.81, 126.33, 126.16, 125.49, 125.25, 122.61, 62.70, 43.16, 39.09, 14.08, 12.80. MS EI m/z (rel. int.) 333 (M⁺, 28), 261 (100), 202 (32), 190 (27), 189 (71), 57 (32); HRMS m/z (EI, M⁺) calcd for C₂₂H₂₃NO₂, 333.1729. found 333.1737.

### N,N-Diethyl-1-methoxy-4-(4-methoxyphenyl)-2-naphthamide

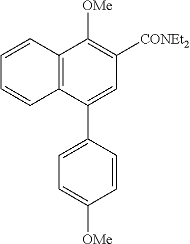

Light yellow solid. mp 129-130° C. (EtOAc/hexanes); IR (KBr) νₘₐₓ 2972, 2935, 1632, 1610, 1515, 1476, 1458, 1430, 1370, 1272, 1248, 1222, 1177, 1062, 1033, 839, 773 cm⁻¹; ¹H NMR (400 MHz, CDCl₃) δ ppm 8.23 (d, J=8.3 Hz, 1H), 7.91 (d, J=8.3 Hz, 1H), 7.55 (t, J=7.5 Hz, 1H), 7.47 (t, J=7.6 Hz, 1H), 7.40 (d, J=8.6 Hz, 2H), 7.25 (s, 1H), 7.02 (d, J=8.6 Hz, 2H), 4.05 (s, 3H), 3.88 (s, 3H), 3.85-3.73 (m, 1H), 3.57-3.39 (m, 1H), 3.37-3.11 (m, 2H), 1.31 (t, J=7.1 Hz, 3H), 1.07 (t, J=7.1 Hz, 3H); ¹³C NMR (101 MHz, CDCl₃) δ ppm 168.95, 158.97, 150.88, 136.34, 133.18, 132.22, 131.10 (2C), 128.02, 126.73, 126.41, 126.13, 125.42, 125.35, 122.61, 113.71 (2C), 62.74, 55.32, 43.17, 39.09, 14.11, 12.83. MS EI m/z (rel. int.) 363 (M⁺, 36), 291 (100), 205 (24), 189 (47), 177 (27), 176 (33), 56 (33); HRMS m/z (EI, M⁺) calcd for C₂₃H₂₅NO₃, 363.1834. found 363.1834.

### 2-Methoxy-6-(4-methoxyphenyl)-N,N-dimethyl-1-naphthamide

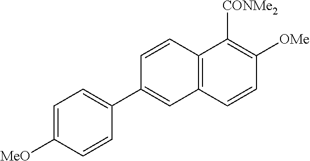

Light yellow solid. mp 187-188° C. (EtOAc/hexanes); IR (KBr) νₘₐₓ 1621, 1503, 1455, 1394, 1284, 1258, 1190, 1136, 1071, 1043, 1029, 841, 817, 705 cm⁻¹; ¹H NMR (400 MHz, CDCl₃) δ ppm 7.93 (s, 1H), 7.89 (d, J=9.0 Hz, 1H), 7.71 (d, J=8.8 Hz, 1H), 7.67 (d, J=8.8 Hz, 1H), 7.62 (d, J=8.5 Hz, 2H), 7.28 (d, J=9.0 Hz, 1H), 7.01 (d, J=8.5 Hz, 2H), 3.96 (s, 3H), 3.86 (s, 3H), 3.28 (s, 3H), 2.83 (s, 3H); ¹³C NMR (101 MHz, CDCl₃) δ ppm 168.66, 159.12, 152.45, 136.36, 133.27, 130.44, 129.60, 129.14, 128.16 (2C), 126.94, 125.02, 124.34, 119.88, 114.27 (2C), 113.28, 56.46, 55.32, 37.82, 34.58. MS EI m/z (rel. int.) 335 (M⁺, 41), 291 (100), 233 (22), 189 (24), 176 (23); HRMS m/z (ESI, [M+1]⁺) calcd for C₂₁H₂₂NO₃, 336.1599. found 336.1588.

### 2-Methoxy-6-(4-methoxyphenyl)-N,N-diethyl-1-naphthamide

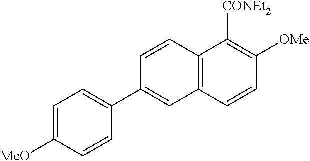

Light yellow solid. mp 118-120° C. (EtOAc/hexanes); IR (KBr) νₘₐₓ 2973, 2936, 1630, 1519, 1499, 1461, 1439, 1285, 1255, 1177, 1075, 1033, 820 cm⁻¹; ¹H NMR (400 MHz, CDCl₃) δ ppm 7.93 (s, 1H), 7.88 (d, J=9.0 Hz, 1H), 7.76-7.66 (m, 2H), 7.63 (d, J=8.7 Hz, 2H), 7.28 (d, J=9.0 Hz, 1H), 7.01 (d, J=8.7 Hz, 2H), 3.95 (s, 3H), 3.92-3.80 (m, 1H), 3.87 (s, 3H), 3.67-3.55 (m, 1H), 3.21-3.06 (m, 2H), 1.38 (t, J=7.1 Hz, 3H), 0.99 (t, J=7.1 Hz, 3H); ¹³C NMR (101 MHz, CDCl₃) δ ppm 167.80, 159.12, 152.32, 136.30, 133.33, 130.18, 129.83, 129.13, 128.17 (2C), 126.86, 125.00, 124.30, 120.43, 114.28 (2C), 113.33, 56.36, 55.35, 42.83, 38.83, 14.04, 13.08. MS EI m/z (rel. int.) 363 (M⁺, 39), 291 (100), 276 (15), 233 (24), 189 (25); HRMS m/z (EI, M⁺) calcd for C₂₃H₂₅NO₃, 363.1834. found 363.1830.

### N,N-Diethyl-1,4-diphenyl-2-naphthamide

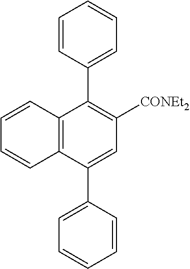

Light yellow solid. mp 111-113° C. (EtOAc/hexanes); IR (KBr) νₘₐₓ 2973, 2932, 1631, 1475, 1460, 1430, 1380, 1272, 1106, 772, 754, 733, 702 cm⁻¹; ¹H NMR (400 MHz, CDCl₃) δ ppm 7.96 (dd, J=7.4, 1.7 Hz, 1H), 7.76 (dd, J=7.1, 1.9 Hz, 1H), 7.61 (d, J=7.3 Hz, 1H), 738-7.32 (m, 12H), 3.88-3.70 (m, 1H), 3.31-3.12 (m, 1H), 2.94-2.68 (m, 2H), 0.91 (t, J=7.1 Hz, 3H), 0.71 (t, J=7.1 Hz, 3H); ¹³C NMR (101 MHz, CDCl₃) δ ppm 170.07, 140.47, 140.07, 137.23, 134.97, 133.82, 132.40, 131.69, 131.28, 130.04 (2C), 129.81, 128.65, 128.31 (2C), 127.66, 127.49, 127.35, 126.85, 126.34, 126.27, 126.18, 124.20, 42.39, 37.86, 13.80, 11.76. MS EI m/z (rel. int.) 379 (M⁺, 32), 378 (22), 308 (33), 307 (100), 278 (35), 277 (43), 276 (59), 202 (30), 77 (50), 57 (46), 56 (65); HRMS m/z (ESI, [M+1]⁺) calcd for C₂₇H₂₆NO, 380.2014. found 380.1997.

### N,N-Diethyl-4-(4-methoxyphenyl)-1-phenyl-2-naphthamide

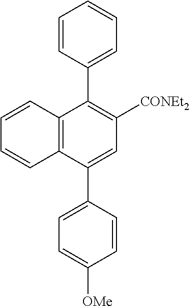

Light yellow solid. mp 126-128° C. (EtOAc/hexanes); IR (KBr) νₘₐₓ 2972, 1631, 1610, 1515, 1505, 1475, 1460, 1433, 1380, 1290, 1272, 1247, 1178, 1107, 1033, 838, 771, 733, 702 cm⁻¹; ¹H NMR (400 MHz, CDCl₃) δ ppm 7.99 (d, J=7.4 Hz, 1H), 7.75 (dd, J=7.6, 1.3 Hz, 1H), 7.60 (d, J=7.3 Hz, 1H), 7.55-7.30 (m, 9H), 7.06 (d, J=8.5 Hz, 2H), 3.91 (s, 3H), 3.85-3.71 (m, 1H), 3.33-3.11 (m, 1H), 2.94-2.67 (m, 2H), 0.91 (t, J=7.0 Hz, 3H), 0.70 (t, J=7.0 Hz, 3H); ¹³C NMR (101 MHz, CDCl₃) δ ppm 170.14, 159.11, 140.15, 137.29, 134.64, 133.84, 132.44, 132.41, 131.89, 131.29, 131.12 (2C), 129.83, 128.64, 127.61, 127.33, 126.83, 126.27, 126.22, 126.16, 124.16, 113.78 (2C), 55.33, 42.37, 37.83, 13.79, 11.75. MS/MS ESI m/z (rel. int.) 410 ([M+1]⁺, 100), 337 (77), 100 (49), 72 (19); HRMS m/z (ESI, [M+1]⁺) calcd for C₂₈H₂₈NO₂, 410.2120. found 410.2109.

### 2-Phenyl-6-(4-methoxyphenyl)-N,N-dimethyl-1-naphthamide

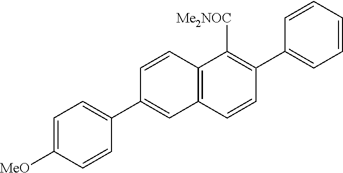

Light yellow solid, mp 171-173° C. (EtOAc/hexanes); IR (KBr) νₘₐₓ 2931, 1632, 1609, 1519, 1463, 1445, 1401, 1285, 1247, 1182, 1028, 826, 789, 761, 730, 700 cm⁻¹; ¹H NMR (400 MHz, CDCl₃) δ ppm 8.03 (s, 1H), 7.95 (d, J=8.8 Hz, 1H), 7.93 (d, J=9.0 Hz, 1H), 7.78 (d, J=8.7 Hz, 1H), 7.67 (d, J=8.6 Hz, 2H), 7.60 (d, J=7.1 Hz, 2H), 7.54 (d, J=8.4 Hz, 1H), 7.45 (t, J=7.3 Hz, 2H), 7.38 (t, J=7.3 Hz, 1H), 7.04 (d, J=8.6 Hz, 2H), 3.88 (s, 3H), 3.00 (s, 3H), 2.45 (s, 3H); ¹³C NMR (101 MHz, CDCl₃) δ ppm 170.17, 159.38, 140.18, 138.59, 135.47, 133.10, 133.02, 132.23, 129.08, 128.86 (2C), 128.82, 128.36 (4C), 127.69, 127.57, 126.68, 126.05, 124.95, 114.35 (2C), 55.36, 37.72, 34.48. MS EI m/z (rel. int.) 381 (M⁺, 60), 338 (28), 337 (100), 319 (25), 276 (25), 265 (37), 263 (43), 239 (24), 169 (21), 132 (24), 77 (32), 72 (27); HRMS m/z (ESI, [M+1]⁺) calcd for C₂₆H₂₄NO₂, 382.1807. found 382.1822.

### 2-Phenyl-6-(4-methoxyphenyl)-N,N-diethyl-1-naphthamide

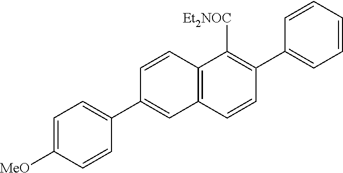

Light yellow solid. mp 147-148° C. (EtOAc/hexanes); IR (KBr) νₘₐₓ 2973, 2933, 1625, 1519, 1494, 1460, 1440, 1284, 1269, 1248, 1181, 1034, 836, 825, 789, 760, 702 cm⁻¹; ¹H NMR (400 MHz, CDCl₃) δ ppm 8.02 (s, 1H), 7.94 (d, J=7.4 Hz, 1H), 7.92 (d, J=7.8 Hz, 1H), 7.77 (d, J=8.7 Hz, 1H), 7.68 (d, J=8.4 Hz, 2H), 7.63 (d, J=7.1 Hz, 2H), 7.53 (d, J=8.5 Hz, 1H), 7.42 (t, J=7.0 Hz, 2H), 7.36 (t, J=7.0 Hz, 1H), 7.03 (d, J=8.4 Hz, 2H), 3.88 (s, 1H), 3.86-3.75 (m, 1H), 3.33-3.12 (m, 1H), 3.04-2.87 (m, 1H), 2.75-2.59 (m, 1H), 1.00 (t, J=7.0 Hz, 3H), 0.65 (t, J=7.0 Hz, 3H); ¹³C NMR (101 MHz, CDCl₃) δ ppm 169.29, 159.36, 140.11, 138.46, 135.07, 133.11, 133.00, 132.70, 129.28 (2C), 129.04, 128.79, 128.33 (2C), 128.24 (2C), 127.75, 127.48, 126.52, 126.11, 124.91, 114.33 (2C), 55.36, 42.43, 38.30, 13.62, 12.12. MS EI m/z (rel. int.) 409 (M⁺, 46), 338 (32), 337 (100), 265 (41), 263 (38), 239 (41), 202 (45), 77 (40), 72 (47), 56 (42); HRMS m/z (EI, M⁺) calcd for C₂₈H₂₇NO₂, 409.2042. found 409.2018.

### Methyl 2-o-tolyl-1-naphthoate

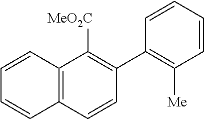 [SMILES: COC(=O)c1c(-c2ccccc2C)ccc2ccccc12]

Light yellow oil. IR (KBr) νₘₐₓ 2949, 1727, 1492, 1435, 1279, 1256, 1234, 1137, 1031, 1018, 827, 760, 728 cm⁻¹; ¹H NMR (400 MHz, CDCl₃) δ ppm 7.97 (d, J=7.8 Hz, 1H), 7.94 (d, J=8.5 Hz, 1H), 7.92 (dd, J=7.4, 1.5 Hz, 1H), 7.62-7.51 (m, 2H), 7.37 (d, J=8.4 Hz, 1H), 7.32-7.26 (m, 2H), 7.25-7.17 (m, 2H), 3.59 (s, 3H), 2.17 (s, 3H); ¹³C NMR (101 MHz, CDCl₃) δ ppm 169.41, 140.19, 138.30, 136.02, 132.26, 130.54, 129.88, 129.83, 129.42, 129.12, 128.13, 127.75, 127.46, 127.32, 126.26, 125.24, 125.11, 51.85, 20.16. MS EI m/z (rel. int.) 276 (M⁺, 40), 245 (71), 244 (24), 217 (28), 216 (51), 215 (100), 213 (24), 202 (41), 189 (19); HRMS m/z (EI, M⁺) calcd for C₁₉H₁₆O₂, 276.1150. found 276.1150.

### Methyl 2-p-tolyl-1-naphthoate

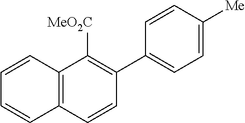 [SMILES: COC(=O)c1c(-c2ccc(C)cc2)ccc2ccccc12]

Colorless solid. mp 109-111° C. (EtOAc/hexanes); IR (KBr) νₘₐₓ 2948, 1725, 1504, 1435, 1286, 1234, 1148, 1137, 1032, 813, 749 cm¹; ¹H NMR (400 MHz, CDCl₃) δ ppm 7.94 (d, J=8.4 Hz, 2H), 7.88 (d, J=7.8 Hz, 1H), 7.59-7.46 (m, 3H), 7.38 (d, J=7.9 Hz, 2H), 7.24 (d, J=7.8 Hz, 2H), 3.73 (s, 3H), 2.41 (s, 3H); ¹³C NMR (101 MHz, CDCl₃) δ ppm 170.17, 137.93, 137.89, 137.34, 132.17, 129.98, 129.85, 129.76, 129.17 (2C), 128.34 (2C), 128.08, 127.50, 127.35, 126.18, 124.98, 52.17, 21.19. MS EI m/z (rel. int.) 276 (M⁺, 75), 245 (100), 244 (29), 215 (50), 202 (81); HRMS m/z (EI, M⁺) calcd for C₁₉H₁₆O₂, 276.1150. found 276.1165.

### Methyl 2-(3-(tert-butoxymethyl)phenyl)-1-naphthoate

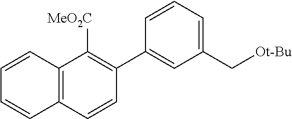 [SMILES: COC(=O)c1c(-c2cccc(COC(C)(C)C)c2)ccc2ccccc12]

Light yellow oil. IR (KBr) ν₁, 2973, 1724, 1435, 1363, 1235, 1193, 1138, 1072, 1032, 790, 749 cm⁻¹; ¹H NMR (400 MHz, CDCl₃) δ ppm 7.97 (d, J=8.5 Hz, 1H), 7.96 (d, J=8.4 Hz, 1H), 7.90 (dd, J=7.8, 1, 0 Hz, 1H), 7.61-7.50 (m, 3H), 7.49 (s, 1H), 7.45-7.33 (m, 3H), 4.51 (s, 2H), 3.72 (s, 3H), 1.32 (s, 9H); ¹³C NMR (101 MHz, CDCl₃) δ ppm 170.05, 140.73, 140.25, 138.04, 132.27, 129.98, 129.87, 128.38, 128.09, 127.56, 127.47, 127.38, 127.27, 126.62, 126.25, 125.05, 73.51, 63.99, 52.24, 27.69 (3C) (1C not observed). MS EI m/z (rel. int.) 348 (M⁺, 28), 275 (23), 245 (36), 231 (54), 215 (44), 203 (30), 202 (100), 201 (29), 200 (33), 189 (25), 57 (50); HRMS m/z (EI, M⁺) calcd for C₁₆H₁₂O₃, 348.1725. found 348.1730.

### Methyl 2-(4-(trifluoromethyl)phenyl)-1-naphthoate

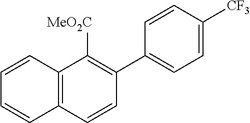 [SMILES: COC(=O)c1c(-c2ccc(C(F)(F)F)cc2)ccc2ccccc12]

Colorless solid. mp 74-76° C. (EtOAc/hexanes); IR (KBr) νₘₐₓ 1728, 1325, 1237, 1167, 1125, 1114, 1085, 1064, 1022, 820 cm⁻¹; ¹H NMR (400 MHz, CDCl₃) δ ppm 8.04-7.95 (m, 2H), 7.92 (dd, J=7.5, 1.4 Hz, 1H), 7.71 (d, J=8.1 Hz, 2H), 7.65-7.54 (m, 4H), 7.49 (d, J=8.5 Hz, 1H), 3.72 (s, 3H); ¹³C NMR (101 MHz, CDCl₃) δ ppm 169.55, 144.57, 144.56, 136.52, 132.60, 130.25, 129.89, 129.76 (q, ²J_{C-F}=32.52 Hz), 128.90, 128.18, 127.75, 126.84, 126.79, 125.35 (q, ³J_{C-F}=3.74 Hz, 2C), 125.17, 124.17 (q, =272.07 Hz), 52.28. MS EI m/z (rel. int.) 330 (M⁺, 62), 299 (100), 251 (29), 202 (65), 69 (65); HRMS m/z (EI, M⁺) calcd for C₁₉H₁₃F₃O₂, 330.0868. found 330.0848.

### Methyl 2-(3-methoxyphenyl)-1-naphthoate

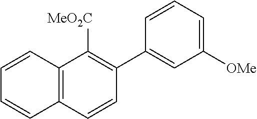 [SMILES: COC(=O)c1c(-c2cccc(OC)c2)ccc2ccccc12]

Colorless viscous oil. IR (KBr) νₘₐₓ 1723, 1608, 1595, 1582, 1466, 1435, 1293, 1236, 1138, 1047, 1032, 787 cm⁻¹; ¹H NMR (400 MHz, CDCl₃) δ ppm 7.96 (d, J=8.1 Hz, 2H), 7.90 (d, J=7.9 Hz, 1H), 7.63-7.48 (m, 3H), 7.36 (t, J=7.7 Hz, 1H), 7.08 (d, J=7.6 Hz, 1H), 7.05 (s, 1H), 6.94 (d, J=8.2 Hz, 1H), 3.85 (s, 3H), 3.74 (s, 3H); ¹³C NMR (101 MHz, CDCl₃) δ ppm 170.00, 159.55, 142.22, 137.82, 132.32, 129.89 (3C), 129.43, 128.09, 127.43, 127.26, 126.33, 125.02, 120.95, 113.86, 113.45, 55.25, 52.23. MS EI m/z (rel. int.) 292 (M⁺, 75), 261 (94), 260 (29), 218 (28), 202 (34), 190 (25), 189 (100), 188 (25); HRMS m/z (EI, M⁺) calcd for C₁₉H₁₆O₃, 292.1099. found 292.1092.

### Methyl 2-(4-methoxyphenyl)-1-naphthoate

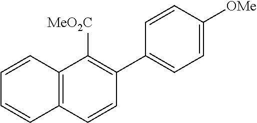 [SMILES: COC(=O)c1c(-c2ccc(OC)cc2)ccc2ccccc12]

Light yellow solid. mp 115-116° C. (EtOAc/hexanes); IR (KBr) νₘₐₓ 1724, 1610, 1518, 1504, 1463, 1435, 1292, 1242, 1180, 1137, 1032, 821, 750 cm⁻¹; ¹H NMR (400 MHz, CDCl₃) δ ppm 7.94 (d, J=8.3 Hz, 2H), 7.89 (d, J=7.9 Hz, 1H), 7.61-7.47 (m, 3H), 7.43 (d, J=8.5 Hz, 2H), 6.99 (d, J=8.5 Hz, 2H), 3.87 (s, 3H), 3.75 (s, 3H); ¹³C NMR (101 MHz, CDCl₃) δ ppm 170.24, 159.19, 137.54, 133.20, 132.06, 129.99, 129.82, 129.63 (3C), 128.07, 127.51, 127.36, 126.11, 124.90, 113.91 (2C), 55.27, 52.20. MS EI m/z (rel. int.) 292 (M⁺, 55), 261 (93), 260 (28), 218 (21), 202 (19), 190 (31), 189 (100); HRMS m/z (EI, M⁺) calcd for C₁₉H₁₆O₃, 292.1099. found 292.1089.

### Methyl 2-(2-fluorophenyl)-1-naphthoate

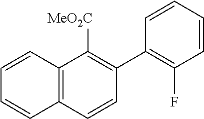 [SMILES: COC(=O)c1c(-c2ccccc2F)ccc2ccccc12]

Light yellow oil. IR (KBr) νₘₐₓ 1725, 1497, 1464, 1450, 1435, 1276, 1236, 1213, 1139, 1034, 1018, 827, 809, 759 cm⁻; ¹H NMR (400 MHz, CDCl₃) δ ppm 8.06 (d, J=7.9 Hz, 1H), 7.97 (d, J=8.5 Hz, 1H), 7.91 (dd, J=7.4, 1.73 Hz, 1H), 7.64-7.53 (m, 2H), 7.51 (dd, J=8.5, 1.34 Hz, 1H), 7.43-7.32 (m, 2H), 7.24-7.12 (m, 2H), 3.70 (s, 3H); ¹³C NMR (101 MHz, CDCl₃) δ ppm 169.17, 159.56 (d, J_{C-F}=247.0 Hz, 1C), 132.67, 132.56, 131.05 (d, J_{C-F}=3.1 Hz, 1C), 130.71, 130.04, 129.87, 129.63 (d, J_{C-F}=8.0 Hz, 1C), 128.36 (d, J_{C-F}=15.7 Hz, 1C), 128.14, 127.75 (d, J_{C-F}=1.4 Hz, 1C), 127.44, 126.61, 125.31, 123.96 (d, J=3.7 Hz, 1C), 115.64 (d, J_{C-F}=22.1 Hz, 1C), 52.07. MS EI m/z (rel. int.) 280 (M⁺, 69), 249 (99), 221 (37), 220 (100); HRMS m/z (EI, M⁺) calcd for C₁₈H₁₃F O₂, 280.0900. found 280.0907.

### Methyl 2-(4-fluorophenyl)-1-naphthoate

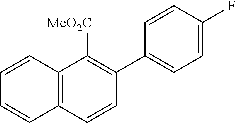 [SMILES: COC(=O)c1c(-c2ccc(F)cc2)ccc2ccccc12]

Light yellow solid. mp 113-114° C. (EtOAc/hexanes); IR (KBr) νₘₐₓ 1726, 1606, 1514, 1505, 1435, 1266, 1235, 1161, 1138, 1032, 1020, 852, 844, 821, 809, 749 cm⁻¹; ¹H NMR (400 MHz, CDCl₃) δ ppm 8.02-7.93 (m, 2H), 7.90 (d, J=7.7 Hz, 1H), 7.63-752 (m, 2H), 7.48 (d, J=8.6 Hz, 1H), 7.47-7.41 (m, 2H), 7.14 (t, J=8.7 Hz, 2H), 3.73 (s, 3H); ¹³C NMR (101 MHz, CDCl₃) δ ppm 169.88, 162.44 (d, J_{C-F}=247.1 Hz, 1C), 136.87, 136.84, 132.28, 130.17 (d, J_{C-F}=8.1 Hz, 1C), 130.03, 129.98, 129.89, 128.12, 127.55, 127.22, 126.43, 125.01, 115.38 (d, J_{C-F}=21.5 Hz, 1C), 52.21. MS EI m/z (rel. int.) 280 (M⁺, 62), 249 (100), 221 (36), 220 (93); HRMS m/z (EI, M⁺) calcd for C₁₈H₁₃F O₂, 280.0900. found 280.0887.

### Methyl 2-(naphthalen-2-yl)-1-naphthoate

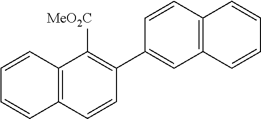 [SMILES: COC(=O)c1c(-c2ccc3ccccc3c2)ccc2ccccc12]

Pale solid. mp 139-140° C. (EtOAc/hexanes); IR (KBr) νₘₐₓ 3056, 1724, 1504, 1434, 1238, 1137, 1032, 820, 744 cm⁻¹; ¹H NMR (400 MHz, CDCl₃) δ ppm 8.03 (d, J=7.5 Hz, 1H), 8.01 (d, J=8.3 Hz, 1H), 7.98 (d, J=1.2 Hz, 1H), 7.96-7.87 (m, 4H), 7.68-7.49 (m, 6H), 3.67 (s, 3H); ¹³C NMR (101 MHz, CDCl₃) 6 ppm 170.03, 138.31, 137.93, 133.32, 132.58, 132.33, 130.15, 130.04, 129.99, 128.18, 128.14, 128.08, 127.68, 127.59, 127.50 (2C), 126.60, 126.38 (2C), 126.23, 125.08, 52.21. MS EI m/z (rel. int.) 312 (M⁺, 78), 282 (20), 281 (94), 280 (24), 253 (36), 252 (100), 250 (53), 126 (37), 125 (20); FIRMS m/z (EI, M⁺) calcd for C₂₂H₁₆O₂, 312.1150. found 312.1156.

### Methyl 2-(furan-3-yl)-1-naphthoate

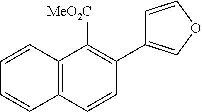 [SMILES: COC(=O)c1c(-c2ccoc2)ccc2ccccc12]

Light yellow oil. IR (KBr) νₘₐₓ 2951, 1769, 1726, 1605, 1509, 1435, 1238, 1152, 1139, 1033, 1019, 829, 752, 749 cm⁻¹; ¹H NMR (400 MHz, CDCl₃) δ ppm 7.91 (d, J=8.5 Hz, 1H), 7.86 (d, J=7.6 Hz, 1H), 7.81 (d, J=8.2 Hz, 1H), 7.67 (s, 1H), 7.59-7.46 (m, 4H), 6.64 (d, J=0.8 Hz, 1H), 3.93 (s, 3H); ¹³C NMR (101 MHz, CDCl₃) δ ppm 170.35, 143.34, 140.11, 132.19, 129.93, 129.84, 129.42, 128.08, 127.91, 127.42, 126.54, 126.26, 124.90, 124.79, 110.56, 52.50. MS EI m/z (rel. int.) 252 (M⁺, 93), 224 (51), 221 (25), 181 (25), 165 (100), 164 (61), 163 (69), 153 (48), 152 (41), 139 (40), 87 (28), 63 (36), 50 (35); HRMS m/z (EI, M⁺) calcd for C₁₆H₁₂O₃, 252.0786. found 252.0786.

### Methyl 2-(thiophen-3-yl)-1-naphthoate

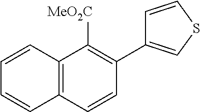 [SMILES: COC(=O)c1c(-c2ccsc2)ccc2ccccc12]

Light yellow oil. IR (KBr) νₘₐₓ 1725, 1435, 1280, 1236, 1137, 1031, 798, 780, 747 cm⁻¹; ¹H NMR (400 MHz, CDCl₃) δ ppm 7.93 (d, J=8.5 Hz, 1H), 7.91-7.84 (m, 2H), 7.61-7.49 (m, 3H), 7.45-7.38 (m, 2H), 7.28 (dd, J=4.6, 1.7 Hz, 1H), 3.83 (s, 3H); ¹³C NMR (101 MHz, CDCl₃) δ ppm 170.29, 140.94, 132.26, 132.16, 129.91, 129.82, 129.69, 128.10, 127.98, 127.46, 126.99, 126.31, 125.92, 124.92, 123.01, 52.41. MS EI m/z (rel. int.) 268 (M⁺, 43), 237 (56), 209 (24), 208 (83), 165 (66), 164 (47), 163 (100), 162 (25), 152 (25), 151 (31), 150 (30), 139 (36), 126 (22), 87 (23), 86 (21), 75 (22), 74 (23), 63 (27); HRMS m/z (EI, M⁺) calcd for C₁₆H₁₂O₂S, 268.0558. found 268.0561.

### Methyl 2-(benzofuran-2-yl)-1-naphthoate

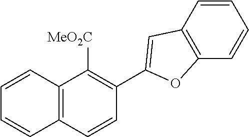 [SMILES: COC(=O)c1c(-c2cc3ccccc3o2)ccc2ccccc12]

Light yellow solid. mp 111-112° C. (EtOAc/hexanes); IR (KBr) νₘₐₓ 1731, 1449, 1434, 1278, 1257, 1238, 1176, 1138, 1079, 1032, 809, 750 cm⁻¹; ¹H NMR (400 MHz, CDCl₃) δ ppm 7.96 (d, J=8.7 Hz, 1H), 7.93-7.83 (m, 3H), 7.63 (d, J=7.5 Hz, 1H), 7.61-7.49 (m, 3H), 7.33 (td, J=7.7, 1.30 Hz, 1H), 7.27 (td, J=7.4, 0.9 Hz, 1H), 7.10 (s, 1H), 4.05 (s, 3H); ¹³C NMR (101 MHz, CDCl₃) δ ppm 170.01, 155.16, 154.19, 132.92, 129.98, 129.90, 128.79, 128.62, 128.13, 127.70, 126.97, 125.20, 125.15, 124.88, 124.11, 123.15, 121.29, 111.17, 104.85, 52.74. MS EI m/z (rel. int.) 302 (M⁺, 92), 271 (44), 231 (31), 215 (75), 214 (28), 213 (100), 202 (34), 189 (29), 187 (33), 163 (26), 126 (47), 63 (30); HRMS m/z (EI, M⁺) calcd for C₂₀H₁₄O₃, 302.0943. found 302.0930.

### (E)-Methyl 2-styryl-1-naphthoate

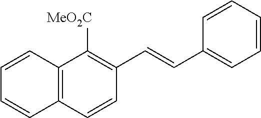 [SMILES: COC(=O)c1c(/C=C/c2ccccc2)ccc2ccccc12]

Light yellow solid. mp 68-71° C. (EtOAc/hexanes); IR (KBr) νₘₐₓ 3058, 2950, 1726, 1509, 1448, 1435, 1283, 1251, 1229, 1215, 1160, 1136, 1035, 957, 8133, 741, 692 cm⁻¹; ¹H NMR (400 MHz, CDCl₃) δ ppm 7.90 (d, J=8.7 Hz, 1H), 7.86-7.79 (m, 3H), 7.58-7.47 (m, 4H), 7.40 (t, J=7.5 Hz, 2H), 7.33 (d, J=16.0 Hz, 1H), 7.31 (t, J=7.3 Hz, 1H), 7.24 (d, J=16.3 Hz, 1H), 4.11 (s, 3H); ¹³C NMR (101 MHz, CDCl₃) δ ppm 169.89, 136.98, 132.54, 132.51, 132.02, 130.12, 129.97, 129.83, 128.73 (2C), 128.14, 128.09, 127.37, 126.79 (2C), 126.28, 125.47, 125.07, 122.66, 52.46. MS EI m/z (rel. int.) 288 (M⁺, 58), 257 (25), 256 (38), 229 (80), 228 (100), 227 (48), 226 (79), 202 (29), 126 (25); HRMS m/z (EI, M⁺) calcd for C₂₀H₁₆O₂, 288.1150. found 288.1153.

### Methyl 2-(2-phenylcyclopropyl)-1-naphthoate

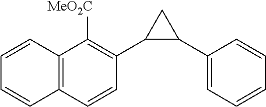 [SMILES: COC(=O)c1c(C2CC2c2ccccc2)ccc2ccccc12]

Light yellow oil. IR (KBr) νₘₐₓ 1725, 1603, 1510, 1498, 1435, 1273, 1231, 1136, 1035, 817, 751, 698 cm⁻¹; ¹H NMR (400 MHz, CDCl₃) δ ppm 7.86 (d, J=8.6 Hz, 1H), 7.83 (d, J=9.2 Hz, 1H), 7.80 (d, J=9.0 Hz, 1H), 7.52 (t, J=7.6 Hz, 1H), 7.46 (t, J=7.5 Hz, 1H), 7.31 (t, J=7.5 Hz, 2H), 7.27 (d, J=8.5 Hz, 1H), 7.23-7.15 (m, 3H), 3.74 (s, 3H), 2.48 (dt, J=8.9, 5.6 Hz, 1H), 2.22 (dt, J=9.0, 5.4 Hz, 1H), 1.58 (dt, J=8.9, 5.7 Hz, 1H), 1.46 (dt, J=8.9, 5.7 Hz, 1H); ¹³C NMR (101 MHz, CDCl₃) δ ppm 170.00, 142.17, 136.94, 131.83, 131.46, 130.01, 129.83, 128.38 (2C), 128.02, 127.22, 125.89, 125.82 (2C), 125.75, 124.41, 123.77, 52.18, 26.67, 26.26, 16.78. MS EI m/z (rel. int.) 302 (M⁺, 2), 196 (28), 183 (89) (25), 165 (50), 152 (41), 139 (58), 127 (48), 126 (44), 115 (70), 104 (100), 103 (39), 91 (93), 89 (37), 78 (82), 77 (73), 63 (34), 51 (36); HRMS m/z (EI, M⁺) calcd for C₂₁H₁₈O₂, 302.1307. found 302.1315.

### N,N-Diethyl-4-methoxy-2-naphthamide

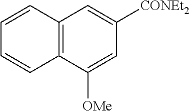

Light yellow oil. IR (KBr) νₘₐₓ 2971, 2935, 1627, 1597, 1577, 1478, 1459, 1422, 1397, 1372, 1293, 1266, 1235, 1111, 1095, 818, 779 cm⁻¹; ¹H NMR (400 MHz, CDCl₃) δ ppm 8.25 (dd, J=6.9, 2.3 Hz, 1H), 7.79 (dd, J=6.8, 2.1 Hz, 1H), 7.59-7.46 (m, 2H), 7.41 (s, 1H), 6.81 (s, 1H), 4.02 (s, 3H), 3.70-3.15 (m, 4H), 1.41-1.08 (m, 6H); ¹³C NMR (101 MHz, CDCl₃) 6 ppm 171.34, 155.65, 134.57, 133.64, 127.80, 127.00, 125.96, 125.60, 121.91, 117.66, 102.16, 55.60, 43.03, 39.00, 14.10, 12.82. MS EI m/z (rel. int.) 257 (M⁺, 85), 242 (40), 186 (32), 185 (100), 158 (32), 157 (47), 114 (22); HRMS m/z (EI, M⁺) calcd for C₁₆H₁₉NO₂, 257.1416. found 257.1424.

### N,N-Diethyl-4-phenyl-2-naphthamide

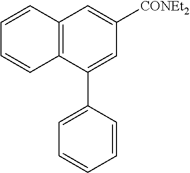

Light yellow solid. mp 123-124° C. (EtOAc/hexanes); IR (KBr) νₘₐₓ 2974, 2934, 1631, 1476, 1462, 1428, 1381, 1271, 1096, 787, 755, 702 cm⁻¹; ¹H NMR (400 MHz, CDCl₃) δ ppm 7.92 (t, J=7.0 Hz, 2H), 7.88 (s, 1H), 7.57-7.40 (m, 8H), 3.76-3.51 (m, 2H), 3.47-3.22 (m, 2H), 1.41-1.23 (m, 3H), 1.21-1.05 (m, 3H); ¹³C NMR (101 MHz, CDCl₃) δ ppm 171.09, 140.74, 139.98, 134.18, 133.25, 131.64, 129.95 (2C), 128.63, 128.29 (2C), 127.49, 126.84, 126.42, 125.98, 125.23, 124.80, 43.39, 39.33, 14.30, 12.97. MS EI m/z (rel. int.) 303 (M⁺, 38), 302 (31), 232 (19), 231 (79), 203 (53), 202 (100), 201 (21), 200 (21); HRMS m/z (ESI, [M+1]⁺) calcd for C₂₁H₂₂NO, 304.1701. found 304.1688.

### N,N-Diethyl-4-(4-methoxyphenyl)-2-naphthamide

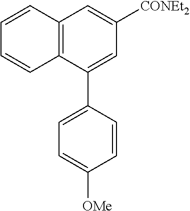

Light yellow solid. mp 147-149° C. (EtOAc/hexanes); IR (KBr) νₘₐₓ, 1430, 1295, 1216, 2974, 2935, 1631, 1515, 1500, 1476, 1462, 1430, 1382, 1287, 1271, 1247, 1178, 1096, 1033, 836, 754 cm⁻¹; ¹H NMR (400 MHz, CDCl₃) δ ppm 7.93 (d, J=8.6 Hz, 1H), 7.91 (d, J=8.3 Hz, 1H), 7.84 (s, 1H), 7.52 (t, J=7.3 Hz, 1H), 7.46 (t, J=7.5 Hz, 1H), 7.43 (d, J=8.5 Hz, 2H), 7.40 (s, 1H), 7.03 (d, J=8.5 Hz, 2H), 3.89 (s, 3H), 3.70-3.49 (m, 2H), 3.44-3.26 (m, 2H), 1.38-1.21 (m, 3H), 1.20-1.04 (m, 3H); ¹³C NMR (101 MHz, CDCl₃) 6 ppm 171.17, 159.10, 140.41, 134.20, 133.29, 132.33, 131.85, 131.04 (2C), 128.63, 126.74, 126.35, 126.02, 124.90, 124.76, 113.75 (2C), 55.34, 43.40, 39.26, 14.34, 12.93. MS EI m/z (rel. int.) 333 (M⁺, 52), 332 (39), 262 (28), 261 (100), 218 (24), 202 (35), 190 (41), 189 (72); HRMS m/z (ESI, [M+1]⁺) calcd for C₂₂H₂₄NO₂, 334.1807. found 334.1797.

### 6-(4-Methoxyphenyl)-N,N-dimethyl-1-naphthamide

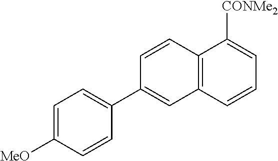

Light yellow solid. mp 113-116° C. (EtOAc/hexanes); IR (KBr) νₘₐₓ 2932, 1635, 1504, 1461, 1395, 1288, 1249, 1179, 1124, 1026, 825, 802, 753 cm⁻¹; ¹H NMR (400 MHz, CDCl₃) δ ppm 8.00 (d, J=1.5 Hz, 1H), 7.89 (d, J=8.2 Hz, 1H), 7.84 (d, J=8.7 Hz, 1H), 7.75 (dd, J=8.7, 1.8 Hz, 4H), 7.65 (d, J=8.8 Hz, 2H), 7.49 (dd, J=8.1, 7.1 Hz, 1H), 7.39 (dd, J=7.0, 1.0 Hz, 4H), 7.02 (d, J=8.8 Hz, 2H), 3.87 (s, 3H), 3.27 (s, 3H), 2.84 (s, 3H); ¹³C NMR (101 MHz, CDCl₃) δ ppm 170.83, 159.38, 138.60, 134.56, 133.82, 133.07, 129.11, 128.38 (2C), 128.33, 126.45, 125.56, 125.36 (2C), 123.59, 114.34 (2C), 55.35, 38.88, 34.86. MS EI m/z (rel. int.) 305 (M⁺, 68), 262 (19), 261 (100), 233 (40), 218 (18), 190 (35), 189 (57); HRMS m/z (ESI, [M+1]⁺) calcd for C₂₀H₂₀NO₂, 306.1494. found 306.1481.

### 6-(4-Methoxyphenyl)-N,N-diethyl-1-naphthamide

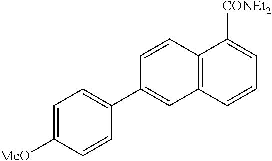

Light yellow oil. IR (KBr) νₘₐₓ 2974, 2934, 1630, 1519, 1501, 1460, 1439, 1426, 1289, 1248, 1181, 1031, 825, 799, 755 cm⁻¹; ¹H NMR (400 MHz, CDCl₃) δ ppm 8.00 (d, J=1.6 Hz, 1H), 7.92-7.82 (m, 2H), 7.75 (dd, J=8.6, 1.8 Hz, 1H), 7.66 (d, J=8.7 Hz, 2H), 7.51-7.44 (m, 1H), 7.38 (dd, J=6.9, 0.89 Hz, 1H), 7.02 (d, J=8.7 Hz, 2H), 4.01-3.75 (m, 1H), 3.87 (s, 3H) 3.65-3.43 (m, 1H), 3.23-3.01 (m, 2H), 1.39 (t, J=7.1 Hz, 3H), 1.02 (t, J=7.1 Hz, 3H); ¹³C NMR (101 MHz, CDCl₃) δ ppm 170.20, 159.35, 138.57, 135.01, 133.81, 133.10, 128.81, 128.44, 128.35 (2C), 126.35, 125.49, 125.29, 125.24, 122.88, 114.32 (2C), 55.34, 43.09, 38.98, 14.29, 13.08. MS EI m/z (rel. int.) 333 (M⁺, 68), 332 (45), 262 (23), 261 (100), 233 (40), 218 (24), 190 (38), 189 (56); HRMS m/z (ESI, [M+1]⁺) calcd for C₂₂H₂₄NO₂, 334.1087. found 334.1797.

We claim:

1-24. (canceled)

25. A compound which is: 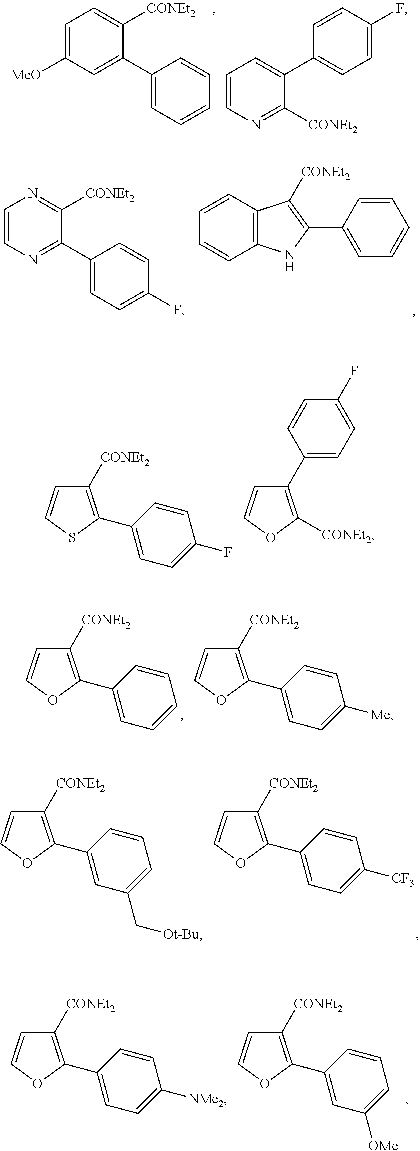 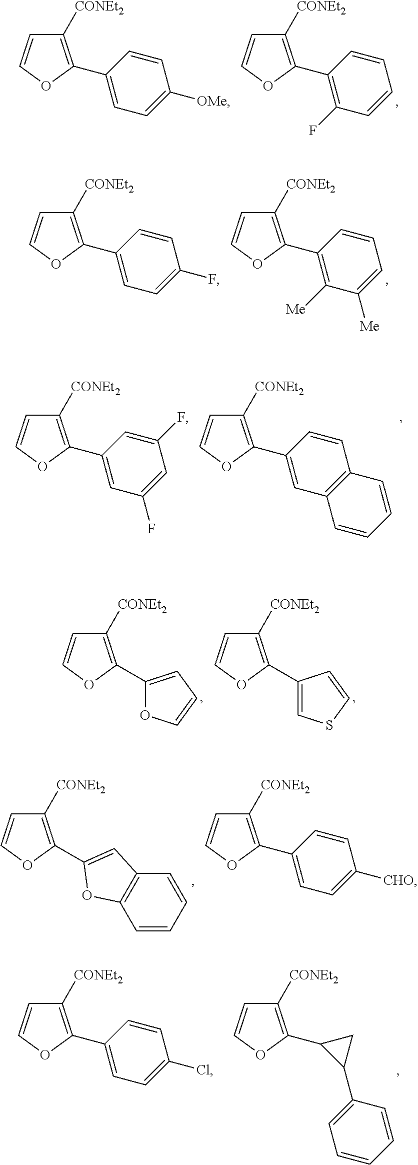 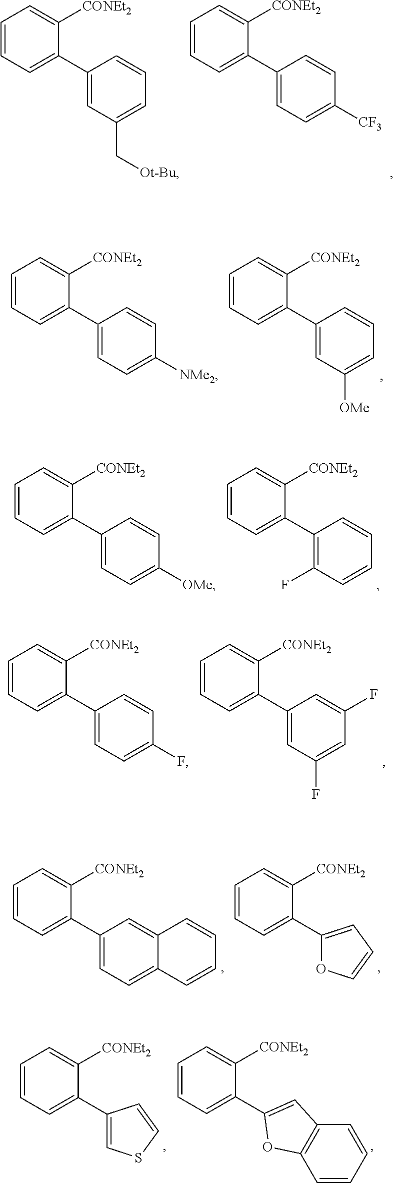 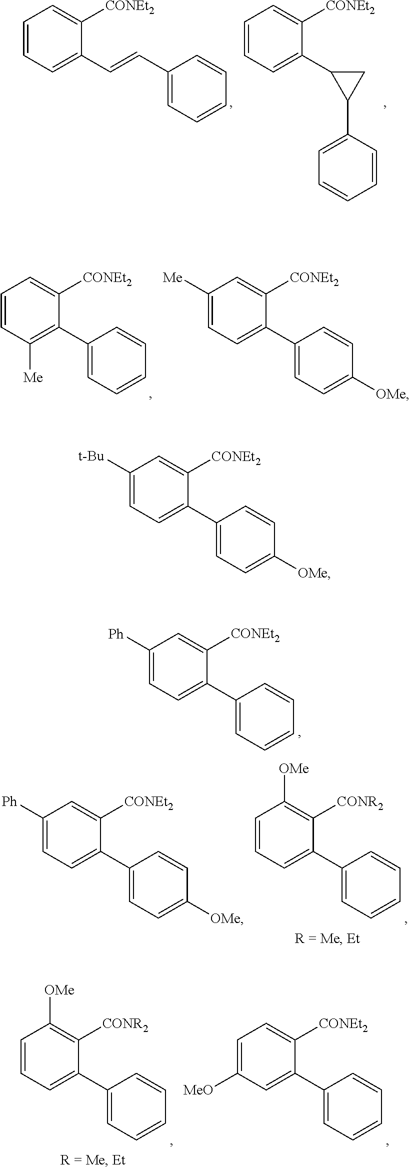 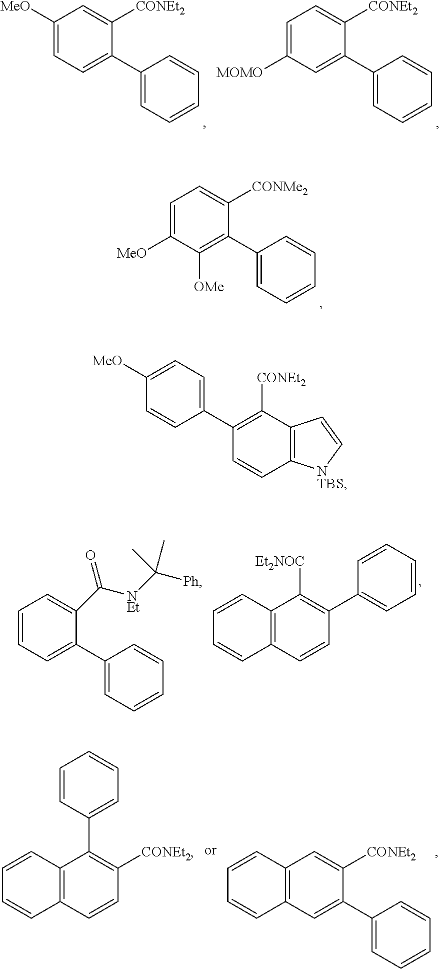
wherein MOM is methoxymethyl, and TBS is tert-butyldimethylsilyl.

26-28. (canceled)

29. A compound which is: 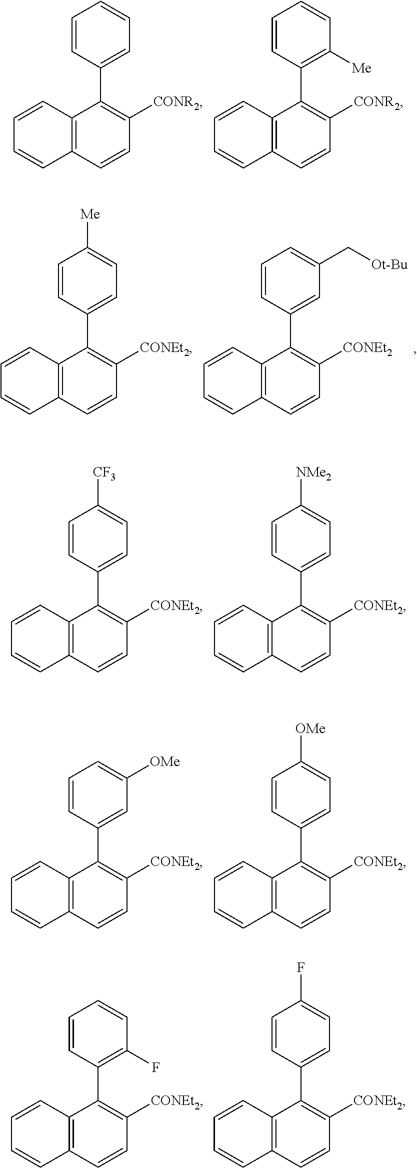 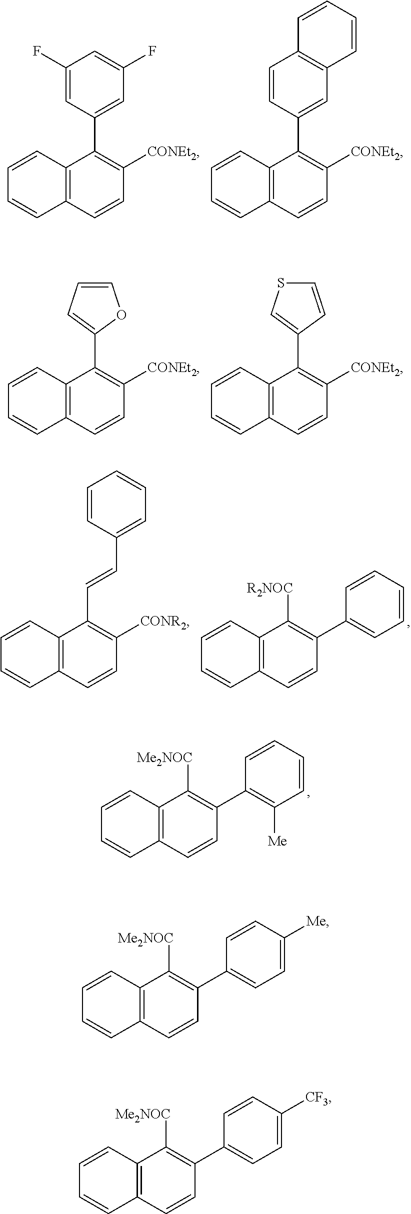 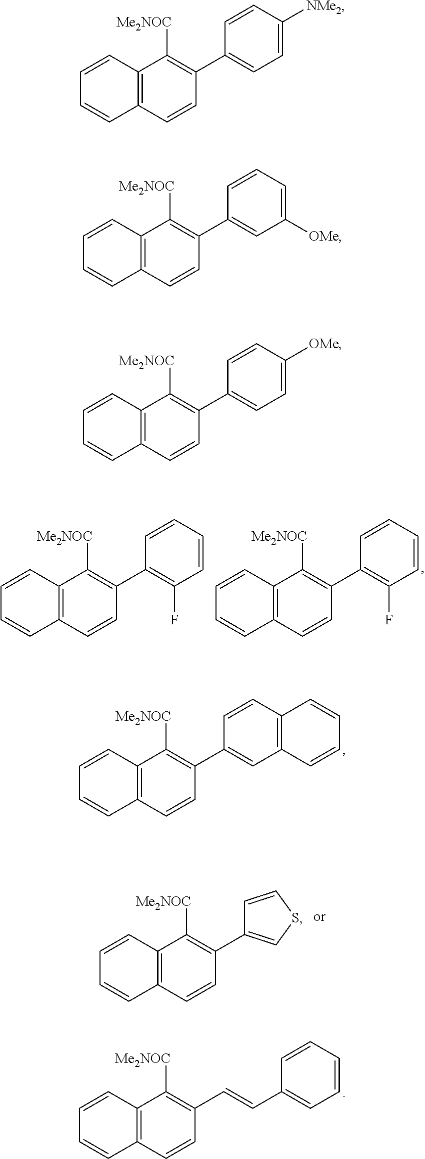
wherein R is Me or Et.

30-31. (canceled)

32. A compound which is:

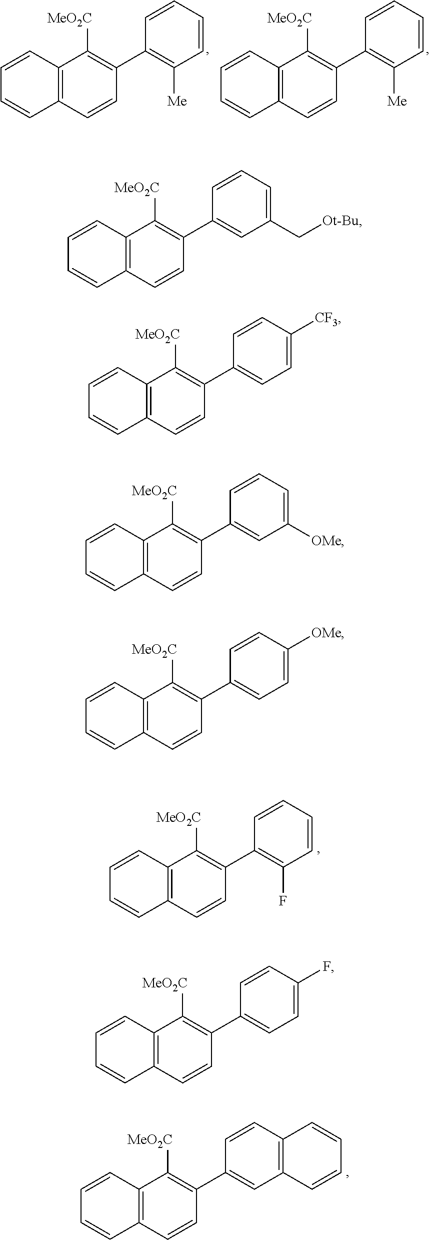

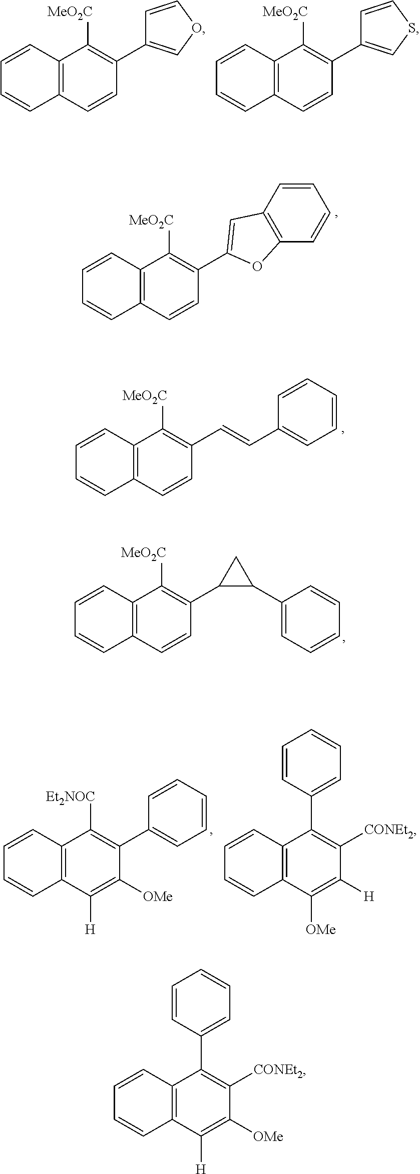

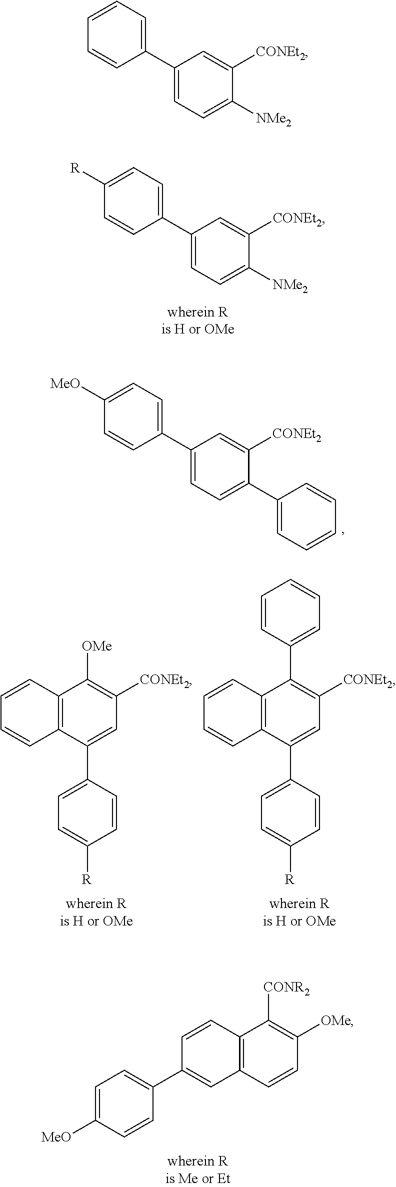

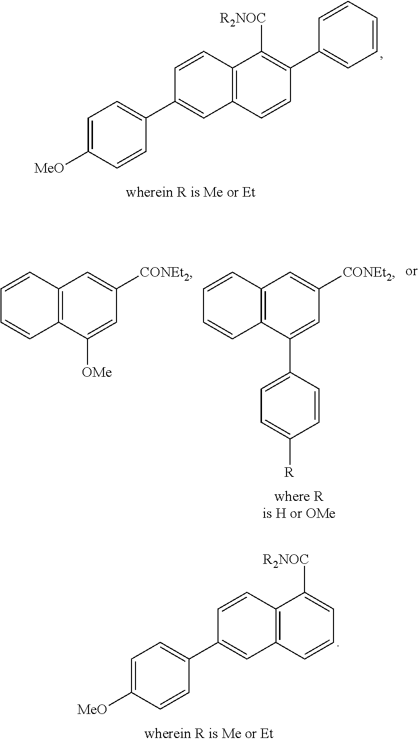

33-77. (canceled)
